SEQUENCE LISTING (52 sequences)

SEQ ID NO: 1 - DNA, 3420 bp, Coriolus hirsutus
catgtcctgtcggctccttgaatgctcgggtctttctcgcgataccgaagtgctgggcaacccggggacgcgtataaagtccaaaaaatcggtctttgacggtgagcgcgacactacgactgcccgccatgaagttcaagagtctcctgttgtccgtgttgccgttggtcggctctggtatgttgcggccttccatccgacaccgagacgagacgctgacagtaacacgccacgaccagtctactcccaggtcgccgcaccctaccaggacgccggcaacggcttcgtctttgacggtgtcactgatccagtgcatagcgtcacgtatggaatcgtcctccctcaggcggcctccagctcggagttcattggcgagatcgtcgcgccaaacgacgcacaatggatcggtttggctcttggaggagccatgatcggcgacctgcttctcgtcgcatggccatatgagaacaaaattattttctcccctcgctacgcgacgtgagtatatgctgttacatgtatgcagacgctacgggctaaatacgccaatctcacagcgggtacacgctgccggcggtctacgaaggcccaaccattaccacactcccgtccagttcgatcaactcgacgcactggaagttcgtgttccgctgccagaactgcacatgtgcgtacctcacattacgtatgacgtctccaactaaacctcttcacagcctgggatggcggaagcattgacccctccggcactggcgtcttcgcgtgggcgtactcgaacgtcgcagtagatacccccgccgatcccaacagcagcttcgccgagcacaccgactgtaagcgatcatctctgaaccatggtactgaatcactcatggtatatcgcagtcggcttcttcggcgtcaacttccccgatgctcagaactcgaactaccaaagctacctccagggcaacgccggcactccccctcccacatccgtccctagcggcccttccagcactacgactactactggtcctacggcaaccgtgagggcttccacttcgctgtgcaggacgttgctaacggtctgtacaggctacgccgtttgactacatcgtcgttggtgccggcccaggtggtctcatcgctgccgatcgcttgtcggaggcgggcaagaaggtccttcttcttgagcgtggtggaccttcgactgcagagaccggcggcacttacgatgtcccatgggccaagtccgctaacgtgagttgaatacccttgaatcgataatgcgcacaccgactgactcccatccatggtagctcacaaaattcgatgtcccgggattgttcgagacgctgttcaccgacacgaacccattctggtggtgcaagggtgggtcggtttctggaagcgcatgtcaacgtcgctaagaaagccttctagacaccaacttctttgctggatgcattctcggtggcggtaccacggtcaacggagcgtaagtgcattgactctgccgtgcccaagcagccctcctgacgacaatctacagtctttactggtaccccaacaacaatgacttctccaccgccagcggatggccgagcagctggaccaaccaccagccgttcaccaacaagctgaagcagcgtctgccgagcacagaccacccctccaccgacggccagcgctacctcgaacagtccgcgaacgtcgtccagcagctgctccagagccagggctaccggcaggtcacgatcaacgacgacccggactccaaggaccacgtcttcggctacagcgcgtttgacttcctcaacgggcagcgcgccggtcccgtcgcgacgtacttccagaccgcgctcgcgcgcaagaacttcgtgtaccgcgacaacgtgctcgtcacgcaggtcatccgcaacggctcgacgatcacgggcgtgcgcacgaacgacctcaccatcgggcccgacggcatcgtgcccctcaacccgaacggccgcgtcatccttgctggcggctcgttcgggaccccgcgcatcctgttccaaagcggcatcgggccgacggacatgctccaggtcgtgcagggcaacgcgcaggccgcggcgaacctgcccccgcagaaccagtggatcaacctcccggtcggccaggccgtgtctgacaacccgtcgatcaacgtgagtgacgctgcatacgcgttcaagcccgccggcctgaggctgacatggctcgtagttggtcttcactcacccgagcatcgacgcgtacgacaactgggcgaccgtatggtcgaacccaaggcaggcggacgctcagcagtacctgcagagccgctccggcgtgttggcgggcgcgtccccgaagtacgttcgacatcgcgcctggagtcttgcaggtgtctgaccagtcctctctacaggctgaacttctggagggcctacggcggcagtgacggtatcacccgctacgtatgtctatgttcgtcttgatctccggtgctacgacctgacattggccgtaggcgcaaggaactgtccgtcctggcgcagcatccgtgaacacctccgttgcgtacaacgcgagccagatcttcacgatcaccctctacctgtgggtaccagcccgaccgtatgtggactgtgcagctaaccgtgcaccactacaggtccaacggtatccagtcgcgcggtcgcatcggcgtggacgccgccctgaacgcgaaggcgctcgtcaacccctggctcaccaacgccgtcgacaagacgatcctgctgcaggccctgcacgacgtcgtctccacactgaataacggtaaggccacttctccgtacctgcctgcgcgcgcgccgctcatgcctctccttcctccagtccaaggcctgacgatgatcacccccgaccacaccatgacgatcgagcagtacgtcgacgcctacgacccggtgagtcccgcccgcagcatcccggcgaaataaaaaacggacgctgacgcccccgtcccacgcaggcgacgatgtgctccaaccactgggtgggcgccgcgaagatcggcacaagcccgtccacggccgtcgtcgacgagaacgcgaaggtgttcaacacggacaacctggtacgtttccctgccctttttcttcccgtgccctccgctgacgcggccttcctgcagttcatcgtcgatgcgtccatcatcccgtctctgccggtcgggaacccgcagggcctgctcatgtctgcggccgagcaggccgtgtcgaagatcctcgcgctcgccggaggaccgtgaggcagggggttcaaaagcatttggagcgctgctatggtagaccatgaagcgggatgggtcctgtcgatatgagacacgatgtatatattatatattctgcacggttttcttcttcctggaagcctgatgaggctctcgacgtgcca

SEQ ID NO: 2 - protein, 768 aa, Coriolus hirsutus
MKFKSLLLSVLPLVGSVYSQVAAPYQDAGNGFVFDGVTDPVHSVTYGIVLPQAASSSEFIGEIVAPNDAQWIGLALGGAMIGDLLLVAWPYENKIIFSPRYATGYTLPAVYEGPTITTLPSSSINSTHWKFVFRCQNCTSWDGGSIDPSGTGVFAWAYSNVAVDTPADPNSSFAEHTDFGFFGVNFPDAQNSNYQSYLQGNAGTPPPTSVPSGPSSTTTTTGPTATATPFDYIVVGAGPGGLIAADRLSEAGKKVLLLERGGPSTAETGGTYDVPWAKSANLTKFDVPGLFETLFTDTNPFWWCKDTNFFAGCILGGGTTVNGALYWYPNNNDFSTASGWPSSWTNHQPFTNKLKQRLPSTDHPSTDGQRYLEQSANVVQQLLQSQGYRQVTINDDPDSKDHVFGYSAFDFLNGQRAGPVATYFQTALARKNFVYRDNVLVTQVIRNGSTITGVRTNDLTIGPDGIVPLNPNGRVILAGGSFGTPRILFQSGIGPTDMLQVVQGNAQAAANLPPQNQWINLPVGQAVSDNPSINLVFTHPSIDAYDNWATVWSNPRQADAQQYLQSRSGVLAGASPKLNFWRAYGGSDGITRYAQGTVRPGAASVNTSVAYNASQIFTITLYLSNGIQSRGRIGVDAALNAKALVNPWLTNAVDKTILLQALHDVVSTLNNVQGLTMITPDHTMTIEQYVDAYDPATMCSNHWVGAAKIGTSPSTAVVDENAKVFNTDNLFIVDASIIPSLPVGNPQGLLMSAAEQAVSKILALAGGP

SEQ ID NO: 3 - DNA, 3480 bp, Coriolus hirsutus
agcgcacgcggcgcgtaccaaatgagcgttcatgtcctgtcggctccttgaatgctcgggtctttctcgcggtaccgaagtgctgggcaacccggggacgcgtataaagtccaaaaaatgggtcttgaacggtgagcacgacactacgaccgcccgccatgaagctcaagagcctcctgttgtccgtgttgccgttggtcggctctggtatgttgcagccttctatctgacatcgagacgagacgctgacagtaacgcaccacgaacagtctacccccaggtcgccgcaccctaccaggacgccggcaacggcttcgtctttgacggtgtcactgacccagtgcatagcgtcacctatggaatcgtcctccctcaggcggcctccagctcggagttcattggcgagatcgtcgcgccaaacgacgcacaatggatcggtttggctcttggaggagccatgatcggcgacctgcttctcgtcgcatggccatatgagaacaaaatcattttctcccctcgctacgcgacgtgagtatatgctgttacatgcatgcagacgctcggggctaaatacgccaatattacagcgggtacaccctgccggcggtctacgacggcccaaccattaccacactcccgtccagttcggtcaactcgacgcactggaagttcgtgtttcgctgccagaactgcacatgtgcgtacctcacatttcgtacgacgtctccaactaaacctcttcacagcctgggatggcggaagcattgacccctccggcactggcgtcttcgcgtgggcgtactcgaacgtcgcagttgatacccccgccgatcccaacagcagcttcgccgagcacaccgactgtaagcaatcatctcttaatcccggtgccgaatcactcatggtatatcgcagtcggcttcttcggcgtcaacttccccgatgctcagaactcgaactaccaaaactacctccagggcaacgccggcaccccccctcccacgtccgtccctagcggcccttccagcactacgactactactggtcctacggcaactgtgagcgcttccacttcactgtgcagaacgtcgctaactttctgtataggctacgccgtttgactacatcgtcgttggtgccggcccaggtggtctcatcgctgccgatcgcctgtcggaggcgggcaagaaggttcttcttcttgagcgtggtggaccttcgacagcagagaccggcggcacttacgatgccccatgggccaagtccgctaacgtgagttgaatacccttgaatcgataatgcgcacaccgactgactcccatccatggtagctcacaaaattcgatgtcccgggattgttcgagacgctgttcaccgacacgaacccattctggtggtgcaagggtgggtcggtttctggaagcgcatgtcaacgtcgctgagaaagcgttctagataccaacttctttgctggatgcattctcggtggcggtaccacggtcaacggagcgtaagtgcatcgactctgccgtgtccaagcagtcctcctaacgacaatctacagtctttactggtaccccaacaacaatgacttctccacggccagcggatggccgagcagctgggccaaccaccagccgttcaccagcaagctgaagcagcgtctgccgagcacagaccacccctccaccgacggccagcgctacctcgaacagtccgcgaacgtcgtccagcagctgcttcaaagccagggctaccggcaggtcacgatcaacgacgacccggactccaaggaccacgtcttcggctacagcgcgttcgacttcctcaacgggcagcgcgccggccccgttgcgacgtacttccagaccgcgctcgcgcgcaagaacttcgtgtaccgcgacaacgtgctcgtcacgcaggtcatccgcaacggctcgacgatcaccggcgtgcgcacgaacgacctcaccatcgggcccgacggcatcgtgcccctcaacccgaacggccgcgtcatcctcgctggcggctcgttcgggaccccgcgcatcctgttccaaagcggcatcgggccgacggacatgctccaggtcgtgcagggcaacgcgcaggctgcggcgaacctgcccccgcagagccagtggatcgacctcccggtcggccaggccgtgtctgacaacccgtcgatcaacgtgagtgacgctgtatacgtgctctagcccgccggcctgaggctgacatggctcgtagttggtcttcacgcacccgagcatcgacgcgtacgacaactgggccaccgtgtggtcgaaccccaggcaggcggacgctcagcagtatctgcagagccgctccggcgtgttggcgggcgcgtccccaaagtacgttcgacatcgtgtccggagtcttgcaggtgtctgaccagtcctctctacaggctgaacttctggagggcctacggcggcagtgacggcatcacccgctacgtatgtctatgtccgtcttcatcaatggaaccgcgatctgacattatccgtaggcgcaaggaaccgtccgtcctggcgcagcatccgtgaacacctccgttgcgtacaacgcgagccagatcttcacgatcaccctctacctgtgggtaccaacccggtcgtatgtataccgtgcagctgaccgtgcgccaccacaggtccaacggtatccagtcgcgcggtcgcattggtgtggacgccgccctgaacgcgaaggcgctcgtcaacccctggctcaccaacgccgtcgacaagacgatcctgctgcaggccctgcacgacgtcgtctccacactgaacaacggtaaggccgcccctacatgcccgcctgcgcgcgccgctcatgccgctccttcctccagtccaaggcctgacgatgatcacccccgaccacaccatgacgatcgagcagtacgtcgacgcctacgacccggtgagtcccgtccgcagcatccccgcaaaagaaaaaaaacgaacgctgacgcccccgtcccacgcaggcgacgatgtgctccaaccactgggtgggcgccgcgaagatcggcacgagcccgtccacggccgtcgtcgacgagaacgcgaaggtgttcaacacggacaacctggtgcgttccccttcgttatgtaactcaccacctcccctggccaccgccgctgacaggatcgacgtttctgcatcgcagttcatcgtcgacgcgtccatcatcccgtctctgccggtcgggaacccgcaggggttgctcatgtccgcggccgagcaggccgtgtcgaagatcctcgcgctcgccggaggaccgtgagggagggggttcaaaagcctttggagcgctgctatggtggaccctgaagcgggatgggttctgtcgatatgagacacgatgtaatattatattctgcatgaatttcttcttcctgcagcctaatgcggactgtctctcgatgtgctaacgagagc

SEQ ID NO: 4 - protein, 768 aa, Coriolus hirsutus
MKLKSLLLSVLPLVGSVYPQVAAPYQDAGNGFVFDGVTDPVHSVTYGIVLPQAASSSEFIGEIVAPNDAQWIGLALGGAMIGDLLLVAWPYENKIIFSPRYATGYTLPAVYDGPTITTLPSSSVNSTHWKFVFRCQNCTSWDGGSIDPSGTGVFAWAYSNVAVDTPADPNSSFAEHTDFGFFGVNFPDAQNSNYQNYLQGNAGTPPPTSVPSGPSSTTTTTGPTATATPFDYIVVGAGPGGLIAADRLSEAGKKVLLLERGGPSTAETGGTYDAPWAKSANLTKFDVPGLFETLFTDTNPFWWCKDTNFFAGCILGGGTTVNGALYWYPNNNDFSTASGWPSSWANHQPFTSKLKQRLPSTDHPSTDGQRYLEQSANVVQQLLQSQGYRQVTINDDPDSKDHVFGYSAFDFLNGQRAGPVATYFQTALARKNFVYRDNVLVTQVIRNGSTITGVRTNDLTIGPDGIVPLNPNGRVILAGGSFGTPRILFQSGIGPTDMLQVVQGNAQAAANLPPQSQWIDLPVGQAVSDNPSINLVFTHPSIDAYDNWATVWSNPRQADAQQYLQSRSGVLAGASPKLNFWRAYGGSDGITRYAQGTVRPGAASVNTSVAYNASQIFTITLYLSNGIQSRGRIGVDAALNAKALVNPWLTNAVDKTILLQALHDVVSTLNNVQGLTMITPDHTMTIEQYVDAYDPATMCSNHWVGAAKIGTSPSTAVVDENAKVFNTDNLFIVDASIIPSLPVGNPQGLLMSAAEQAVSKILALAGGP

SEQ ID NO: 5 - DNA, 19 bp, Artificial Sequence
  modified_base 19: n represents a,g,c or t
taygaraayaavatthttn

SEQ ID NO: 6 - DNA, 20 bp, Artificial Sequence
  Synthetic DNA
gayatcaagttyatcratgg

SEQ ID NO: 7 - DNA, 1496 bp, Coriolus hirsutus
atgttccccacagtctccctcctcgcgttctccctccttgcgaccgtctacggtcagcaagtcggcaccctgacggcggagaaccacccccgcctcaccgtccagcagtgcacggccaagaacaactgccagacccagcagcactccgtcgtgctcgactccaactggcgctggctccacgccaccaccggcagcaacaactgctacaccggcaacacctgggatgcgttgctctgccccgatgcgaccacctgcgccaagaactgcgcggtcgacggcgccgactatgccggtatgtcccgttcttgcggctgcgctgcctgcgacgcgccagttgtctaacacacgcgctcgctaggcacctacggcatcaccacgaacggcaacgcgctcacgctcaagttcgtgcagcagggcccgtactcgaagaacatcggctcgcgcgtgtacctcatggacgcgcaggaccagaagtacgagctcttcaacctgaagaaccaggagttcacgttcgacgtcgacatgtcgaacctcccctgcggcctcaacggcgcgctctacttcgtcgagatggacgccgacggcggcgcgtcccgcttcccgaccaacaaggccggcgcgaagtacggaaccggctactgcgacacccagtgcccgcaggacatcaagttcatcaacggcgtggtaagcaccgacctccccgctgcccgactccccgctgcgtgctcacatccgccccgcaggccaacctcgaggggtgggcgggctcgccctctgacccgaactcgggcaccggcagcttcggcacgtgctgcaacgagatggacgtgtgggaggcgaacaagaacggcgccgcgttcacgccgcacgtctgctccgtcacgagccagacgcgctgcgagggcacgcagtgcggcgacggcgacgagcgctacgacggcctctgcgacaaggacggctgcgacttcaactcgttccgcaagggcgaccagaccttcctcgggccgggcaagaccgtcgacacgaacgcgaagttcacggtcgtcacgcagttcctgacgaacaacaaccagacgtccggccagctgtccgagatccgccgcctgtacgtgcagaacgggcgggtgatcgcgaactcgaagacgaacgtgcccgggctcggcgccttcgactcgatcacggaccagttctgcaacgcgcagaagcaggtgttcggcgacgacaacacgttcgagaagctcggtgggctcaacacgatgggccaggccttccagcgcggcatggcgctcgtcatgtccatctgggacgaccacgccgcgggcatgctctggctcgacgccgactaccccaccgacgcgcccgcgaccaaccccggtgtctcccgtggcccgtgctcggccacctctggcgaccccgcgacgatcgagaacagcgaggcgagctcgtccgtcaccttctcgaacatcaaggtcggccccatcggctcgacgttctga

SEQ ID NO: 8 - protein, 456 aa, Coriolus hirsutus
  modified_amino acid 286: Xaa represents unknown
MFPTVSLLAFSLLATVYGQQVGTLTAENHPRLTVQQCTAKNNCQTQQHSVVLDSNWRWLHATTGSNNCYTGNTWDALLCPDATTCAKNCAVDGADYAGTYGITTNGNALTLKFVQQGPYSKNIGSRVYLMDAQDQKYELFNLKNQEFTFDVDMSNLPCGLNGALYFVEMDADGGASRFPTNKAGAKYGTGYCDTQCPQDIKFINGVANLEGWAGSPSDPNSGTGSFGTCCNEMDVWEANKNGAAFTPHVCSVTSQTRCEGTQCGDGDERYDGLCDKDGCDFNSFRXGDQTFLGPGKTVDTNAKFTVVTQFLTNNNQTSGQLSEIRRLYVQNGRVIANSKTNVPGLGAFDSITDQFCNAQKQVFGDDNTFEKLGGLNTMGQAFQRGMALVMSIWDDHAAGMLWLDADYPPTRPRPTPVVSRGPCSATSGDPATIENSEASSSVTFSNIKVGPIGSTF

SEQ ID NO: 9 - DNA, 1488 bp, Coriolus hirsutus
atgttccccgcagtcgccctcctcgctctctccttcttcgccatcgcctacggccagcaggtcggcacactcacggcggagaaccacccgaagatcacggtccagcagtgcacgggcaagaacagctgccagaccctgcagcgctcggtcgtgctcgactccaactggcgctggctccactcgaccagcggcagcaacaactgctacaccggcaacacctgggacagctccctctgccccgaccccaccacctgcgcgaagaactgcgcgcttgacggcgccgactatgctggtgagcttactcgtcctttgggtctgacgggcgagcgtgctaaccatatttgatgcgttaggcacctacggcatcaccacgagcggtaaccagctcagtctcaagttcgtaacgcacggccagtactcgaccaacatcggctcgcgcgtgtacctcctcgacggtagcgactccaagtaccagcagttcaacctgaagaaccaggagttcacgttcgacatcgacatgtcgaagctcccgtgcggcctcaacggcgcgctctacttcgtcgagatggacgccgacggtggtctctcccgcttcccctccaacaaggctggcgcgaagtacggcaccggctactgcgatacccagtgcccgcacgacatcaagttcatcaacggcgaggtgagttttcaatgagtcaatcttcgtgtgtgccggtgctcacacgtcccttacaggccaacgtcctcggctggacgccctcagacagcgacccgaacgcgggcagcggccagtacggcacgtgctgcaacgagatggacatctgggaggcgaactcgatgggcgcggcggtaactccgcacgtctgctccgtcacgagccagacgcgctgctcgggcacggactgcggcgacggcgacaaccgctacaacggcatctgcgacaaggacggctgcgacttcaactcgtggcgcatgggcgaccagacgttcctcgggccgggcaagaccgtcaacacgaaccagaagttcacggtcgtgacgcagttcctgacgaacaacaaccagacgtcgggcacgctctccgagatccgccgcctgtacgtgcagaacgggaaggtgatcgcgaactcgaagacgaagatccccggcatggacgcgtacgactcgatcaccgacgcgttctgcaacgcgcagaagcaggcgttcggcgacaacaactcgttcgagaggctcggcgggctcaaggcgatgggcgccgccttcgacaagggcatgagcctcgtcatgtccatctgggacgaccacgaggcgaagatgctctggctcgacagcgagtaccccctcgacaaggacgcttccacgcccggtgtctctcgcggcccctgcgcgcggacctccggcgagccgaaggacgtcgagtccaacagccccgacgcgaccgtcgtcttctccaacatcaagtacggccccatcggctcgacctactaa

SEQ ID NO: 10 - protein, 456 aa, Coriolus hirsutus
MFPAVALLALSFFAIAYGQQVGTLTAENHPKITVQQCTGKNSCQTLQRSVVLDSNWRWLHSTSGSNNCYTGNTWDSSLCPDPTTCAKNCALDGADYAGTYGITTSGNQLSLKFVTHGQYSTNIGSRVYLLDGSDSKYQQFNLKNQEFTFDIDMSKLPCGLNGALYFVEMDADGGLSRFPSNKAGAKYGTGYCDTQCPHDIKFINGEANVLGWTPSDSDPNAGSGQYGTCCNEMDIWEANSMGAAVTPHVCSVTSQTRCSGTDCGDGDNRYNGICDKDGCDFNSWRMGDQTFLGPGKTVNTNQKFTVVTQFLTNNNQTSGTLSEIRRLYVQNGKVIANSKTKIPGMDAYDSITDAFCNAQKQAFGDNNSFERLGGLKAMGAAFDKGMSLVMSIWDDHEAKMLWLDSEYPLDKDASTPGVSRGPCARTSGEPKDVESNSPDATVVFSNIKYGPIGSTY

SEQ ID NO: 11 - DNA, 1485 bp, Coriolus hirsutus
atgttccccaccgccgccctcctctcgctctccttcgcggcgatcgcctacggccagcaggtcggcacgctcaccgccgagtcgcaccccaagctcagcgtgcagcaatgcaccgccggcggcagctgccagaccctgcagcgctccgtcgtcctcgactccaactggcgttggctccactctacctcgggctcgaccaactgctacaccggcaacacctgggacgcgtccctctgccccgaccccaccacgtgcgcggcaaactgtgccctcgatggcgctgactactctggtccgcgttgatctcacctgaagacccttgtgcatactcaacaatccctctaggcacctacggtatcaccaccagcggcaacgagctcaacctcaggttcgtcacgaagggccagtactccaccaacatcggctcccgcgtctaccttctctccgaggacgacagcacgtacgagatgttcaacctcaacaaccaggagttcacgttcgacgtcgacatgtcgaacctcccgtgcggcctcaacggcgcgctctacttcgtggagatggacaaggacggtggctcatcccgcttccccaccaacaaggccggctccaagtacggtaccggctactgcgacacccagtgcccgcacgatatcaagttcatcaacggcgaggtaagatgccgcgtctcaacgcagtaaaaccatgctgactcgctcttccgcacgccaggccaacgtcctcggctgggagggctccccgaacgacccgaacgcgggaaccggccagtacggaacgtgctgcaacgagatggacatctgggaggcgaaccagaacggcgcggcggtcacgccgcacgtctgctccgtcgacggccagacgcgctgcgagggcacggactgcggcgacggcgacgagcggtacgacggcatctgcgacaaggacggctgcgacttcaactcgtaccgcatgggcgaccagtccttcctcggcctcggcaagaccgtcgacacctcgaagaagttcaccgtcgtcacccagttcctcaccgcggacaacacgacgaccggccagctcacggagatccgccggctgtacgtgcaggacggcaaggtcatcgcgaactcgaagacgaacatccccggcctcgactcgttcgactccatcaccgacgacttctgcaacgcgcagaaggaggtcttcggcgacaccaactcgttcgagaagctcggcggcctcgcggagatgggcaaggccttccagaagggcatggtcctcgtcatgagcatctgggacgaccacgccgccaacatgctctggctcgacagcgactaccccaccgacgccgacccgtcgaagccaggtgtcgcccgtggcccgtgcccgaccagctctggcgtccccaccgatgtcgagtcgcagagccccaacgcgaacgtcatcttctccaacatcaagaccggccccattggctcgacctacgcttga

SEQ ID NO: 12 - protein, 457 aa, Coriolus hirsutus
MFPTAALLSLSFAAIAYGQQVGTLTAESHPKLSVQQCTAGGSCQTLQRSVVLDSNWRWLHSTSGSTNCYTGNTWDASLCPDPTTCAANCALDGADYSGTYGITTSGNELNLRFVTKGQYSTNIGSRVYLLSEDDSTYEMFNLNNQEFTFDVDMSNLPCGLNGALYFVEMDKDGGSSRFPTNKAGSKYGTGYCDTQCPHDIKFINGEANVLGWEGSPNDPNAGTGQYGTCCNEMDIWEANQNGAAVTPHVCSVDGQTRCEGTDCGDGDERYDGICDKDGCDFNSYRMGDQSFLGLGKTVDTSKKFTVVTQFLTADNTTTGQLTEIRRLYVQDGKVIANSKTNIPGLDSFDSITDDFCNAQKEVFGDTNSFEKLGGLAEMGKAFQKGMVLVMSIWDDHAANMLWLDSDYPTDADPSKPGVARGPCPTSSGVPTDVESQSPNANVIFSNIKTGPIGSTYA

SEQ ID NO: 13 - DNA, 21 bp, Artificial Sequence
  modified_base 9: n represents i
cagtggggngactggtgcaac

SEQ ID NO: 14 - DNA, 1704 bp, Coriolus hirsutus
atgtccaagttcgcgacgctcctcgctctcctgactgtcgtcccctccctggcttacgcccaggcgtcgctgtacggacagtgcggtggtattggcttctgtatgtctcgttttcacgtgcttctgtgagatgcgtactcaccaccgtatcctcgcagcgggtccgacgacttgtgttgccggcgccgtttgcacgaagcagaacgactactactcgcagtgcctgtatgtcgtgtcaaccagttatgagataaagcaacttgctaacttattacgacagtccgggcgccgctgcaccgaccactgttgcaccgaccaccacacctaacgcgcccacatctgcacctggtggtggctcgcccacatcttctgctccgagcgccccttcgagcactcctgctgctggcaaccccttcgacggttttgaggtacagtgttctcgaacaccgcaaatcttcgcatgctcacccatgtatacaacagatctacctgagcccttactacgccaaggaggtcgctgctgccgctgccgccatcaccgatcccacgctgaagagcaaggccgcaagcgttgctaacatcccgactttcacatggctcgactccgtgtcgaaggtgcctgacctcggcacgtacctcgccgacgcgtcctcgatccagagcagcaccggcaagaagcagctcgtgccgatcgtcgtgtacgacctgcccgaccgcgactgcgcggcgaaggcgtccaacggcgagttcagcatcgcggacggcggcgcggcgaagtacaaggactacatcgaccagatcgtcgcgcagatcaagcagttccccgacgtgcgcgtcgtcgcggtcatcgagcccgactcgctcgcgaacctcgtcacgaacctgaacgtgcagaagtgcgcgaacgcggaggcgacgtacaaggccagcgttacgtacgcgctccagcagctctcgtccgtcggcgtgtaccagtacatggacgccggccacgccggctggctcggctggcccgccaacatccagcccgcggcgacccttttcgcggagatgttcaagagcgcgaactcgtcgcctttcgtccgcggtctcgccactagtgagcactcacctagacacagcgatgtggatggccactaacggagcgtcgcagacgtcgccaactacaacgccctgaccgccgcctcccccgacccgatcacccagaacaaccccaactacgacgagtcccactacattaacgtgagtccctcctgctcagcgctgttcagccacaatgctcacattgcacgcacgcacccaggcgctcggcccgatgctcaagtccgccggcttccccgcgcagttcgtcgtcgaccagggccgcgccggccagcagaacctccgccagcagtggggcgactggtgcaacatcaagggcgccggcttcggcacgcgcccgacgacgaacaccggcaacccgctcatcgacgcgatcatctgggtgaagcccggaggcgagtccgacggcacctcgaacagctcgtcgccccgctacgacagcacgtgctctctggtacgtcttcctcgtcctcctgcatctggggggtgcgtgtgcttaccactgcggtgcagtccgacgcgacggtccccgcacccgaggccggtacctggttccaggcgtacttcgagaccctcgtgtccaaggccaacccgccgctgtga

SEQ ID NO: 15 - protein, 453 aa, Coriolus hirsutus
MSKFATLLALLTVVPSLAYAQASLYGQCGGIGFSGPTTCVAGAVCTKQNDYYSQCLPGAAAPTTVAPTTTPNAPTSAPGGGSPTSSAPSAPSSTPAAGNPFDGFEIYLSPYYAKEVAAAAAAITDPTLKSKAASVANIPTFTWLDSVSKVPDLGTYLADASSIQSSTGKKQLVPIVVYDLPDRDCAAKASNGEFSIADGGAAKYKDYIDQIVAQIKQFPDVRVVAVIEPDSLANLVTNLNVQKCANAEATYKASVTYALQQLSSVGVYQYMDAGHAGWLGWPANIQPAATLFAEMFKSANSSPFVRGLATNVANYNALTAASPDPITQNNPNYDESHYINALGPMLKSAGFPAQFVVDQGRAGQQNLRQQWGDWCNIKGAGFGTRPTTNTGNPLIDAIIWVKPGGESDGTSNSSSPRYDSTCSLSDATVPAPEAGTWFQAYFETLVSKANPPL

SEQ ID NO: 16 - DNA, 17 bp, Artificial
  Synthetic DNA
gtaaaacgacggccagt

SEQ ID NO: 17 - DNA, 19 bp, Artificial
  Synthetic DNA
ggaaacagctatgaccatg

SEQ ID NO: 18 - DNA, 1327 bp, Coriolus hirsutus
  Sequence represents cDNA
aagcactctattgacaccgtcatgttctcgtctaccctctccttcgccgccctcgcgctcgcgctcgtcgcgcccactgccgtcaacgctcacggtttcattcatgaatatgagatcggcggcaagagctactccggttggctcccgttctcggacccctacgagagccctgtcccgagccgcatcgagcgcaagatcccgagtgacggccctatccttgatgtcacttctcctgaccttgcctgcaacaaggggggcgagtctggcgtcaaggccatcgccactgcggcagcaggcagccagatcacctttgactggaacagttggcccgcagatcacatgggcccggtgaccacatacatggcgtcttgcaacggtgattgcgcgtctttcgatgcctccaacgcgaagtggttcaagattgacgctgccggctactcgaacggcaagtgggctgccaccaagctcattgagaacggcgccaagtggaccagcaccattcccagcgagctcaaggctggtgaatacttggtccgtcatgagatcattgctctccacgacgccggtgcgcctcagttctaccccagctgcgctcaggtgaaggtcactggtggtggtagccaggttccctctggttcctccctcgtgtccattccggggctctacaccattcaggagttcccgacatctggtccgacagcttcaagagctttgccattcctggacccgcggtcgccttcagtggctccaacagcggctctggcgattctcagcctgctgcctcctcctctacccacgccgctacttcttcggcggcctcccagtctgcgtcctcgacgcaggttcacacctccgcggagacctccgcgcaggcctcggcgacgtctgttgcgtcccacgcatcttccgctgcccacacttcctcggccgcatccgcgtcgaagccctcgtcgacggggaccggaaggtgctcctctaagcgcactcgccgcggcatggtcaagcgcaacgtctctcaccacgccaagcgccaccaccattgatttctctttcttccttgcgctcttggctgtctcgagatctcgatatgcttcagagaagcactggtcgacgggatctcaatcgatgttgatacagatgggttgactcccctccgcgctctgctcccaccgcgccggggatagagtcctcgcgcgcggcttccttaacgttattcattcctcgctccgcataagtctccgcatgctatgtatcggtgctgctagcccgcacgactgcccgacgattgtaccggaatacaacgcgctttgtcctttgtgaaaaaaaa

SEQ ID NO: 19 - protein, 374 aa, Coriolus hirsutus
MFSSTLSFAALALALVAPTAVNAHGFIHEYEIGGKSYSGWLPFSDPYESPVPSRIERKIPSDGPILDVTSPDLACNKGGESGVKAIATAAAGSQITFDWNSWPADHMGPVTTYMASCNGDCASFDASNAKWFKIDAAGYSNGKWAATKLIENGAKWTSTIPSELKAGEYLVRHEIIALHDAGAPQFYPSCAQVKVTGGGSQVPSGSSLVSIPGLYTIQEFPTSGPTASRALPFLDPRSPSVAPTAALAILSLLPPPLPTPLLLRRPPSLRPRRRFTPPRRPPRRPRRRLLRPTHLPLPTLPRPHPRRSPRRRGPEGAPLSALAAAWSSATSLTTPSATTIDFSFFLALLAVSRSRYASEKHWSTGSQSMLIQMG

SEQ ID NO: 20 - DNA, 735 bp, Coriolus hirsutus
gcgggcccgtactcgctcctgctcgaccagtggggcaaggacggcgcgacgtccggctcccaatgcgcgaacctcatcagcctgagcggcagtaccgtcgcgtggaagacgacctggcagtggacgggcggctccggcgtgaagagcttcacgaacatccagctcaacgagggcctcaacaagcagctcagcgcgatcaagagcatccccacgacgtggcagtggtcgcagagcgcgtccgggtcgatcgtcgcggacgtcgcgtacgacctcttcacggcgaacaccgccgggggctcgaacgtgaacgagatcatgatctggctcgcgaacttcaacgcgggcccgatctcgatccagtacggcgcggacggcaagcccgtgcccgtcgcgtcgaacctgagcctcgcgggccacacctggaacctgtacagcggctcgaacggcgcgaacgcggtgttctcgttcctgcccacgagcggcacgattacgagcttcagcggggacgtgaacgtgttcctccagtacttgacgcagcaccagggcgtcagcacctcgcagttcctcgtcaccgcgcaagcgggtacggagcctacatctggctctgcgacgctcacgacgtctgcatacagcttggctatcaactagggagacgaaacatgtacattcagaacttgtgccgacaggaatcacattacttcagacttcccgaaaaaaaaaaaaaaaaaaaaaaaa

SEQ ID NO: 21 - protein, 216 aa, Coriolus hirsutus
  modified_amino acid 216: Xaa represents unknown
AGPYSLLLDQWGKDGATSGSQCANLISLSGSTVAWKTTWQWTGGSGVKSFTNIQLNEGLNKQLSAIKSIPTTWQWSQSASGSIVADVAYDLFTANTAGGSNVNEIMIWLANFNAGPISFQYGADGKPVPVASNLSLAGHTWNLYSGSNGANAVFSFLPTSGTITSFSGDVNVFLQYLTQHQGVSTSQFLVTAQAGTEPTSGSATLTTSAYSLAINX

SEQ ID NO: 22 - DNA, 21 bp, Artificial
  Synthetic DNA
atgaagttacttcttgctctc

SEQ ID NO: 23 - DNA, 21 bp, Artificial
  Synthetic DNA
tcacaggaagggttcgagtgc

SEQ ID NO: 24 - DNA, 1989 bp, Phanerochaete chrysosporium
atgaagtacttcttgctctcagtagcagcgacgcttgcccttagtgcacctgcgcttggtgtcgctgtctggggccaatgcggtgtaagcatatgtgttcatatgagaggcgtacgagaaggttgaccgcgcaacagggaatcggctttagtgggagtactacttgcgatgctggcaatcactgcgtttatctgaacgattgtgaggtcaatgtcatttcaatgccatgatttgctgaccgatatccttgcagattattcgcaatgccagcccggcgcggcgacgaccacggtccggtcgacaagtaccattgcttcaacgacatctaccgcaccatcaagcagcaatagcctttgctccggcagccgcacgaagttcaagtttttcggtgtcaatgagtctggtgctgagttcggtaatctcaacatcccaggtgtcctaggcacggactacacctggccttcgccgtccagcatcgacgtaagtgatacatcatatcagagctgctaagcaggttgctgatagtgatgcactagttcttcatgggcaagggattcaacaccttccgcattccgttcctcatggagcgtatgagccctcccgcgacgggcctcaccggtccgtttgatcagacctatctgagcggtcttcagacggtacgccaaacgtaatagctggaccttgcggggagtaaggctgaccatcaccttacggcagattgtcagctatatcaccggaaaggggggctatgcgctcgtagaccgtgagtgacggctcgcacgcaagtagtaggaggtcatctgatagtgaaggactgcagcgcacaactttatgatctacaatggcacaaccattagcgacacaaacgcgtgagcactcctttagtttcgtctctgcgttagctccgttctgatgatacaacaacacagcttccagacttgtacgtgtgctctgcctgaacccgcttggccgttgatcatcatagtgatgcgttctcacagggtggcacaacctcgccaccgtcttcgtacgtctgtttccatgcgtgaagcctgcaagggtcgcccctgatcggttttatagaaatccaaccccaatgtcgtctttggtgaatgagttcggcgtgtgtgctactcttagcaacgagcttacagcttggcagatgtcatgaacgagccgcacgacattcccgcacagacggtcttcaacctggtgagttgcggccgagtttggggacttctgtcaaactcattcggcgttgtttccccctagaaccaagccgctatcaacggaatccgtgctgcgggtgccacctcgcaacttatccttgttgagggcactagctacactggcgcctggagtgagatcagacccttcacacccgcagaacctgcccattgattgattgtttcgcagcgtggacgacttcttccggtaatggtgctgtctttggtgctatccaagatcccaacaacaatgttgccatcggtgagtgcgggaagcgtaccggtctgccaagtgctcacgggtaagcgtgggaaagagatgcaccagtacctcgacagcgacggctccggcacgtcccccacatgcgtttcgtccacgatcggtgcggagcgtctccaagcggcgacacagtggctgcagcagaacaacctgaaaggcttcctgggtgagatcggagctggttcgaacggtgcgtcggatcatgtcctgtgttgcagcctgtgctgaccgtagtcgttcaaaccagccgactgcatcagtgccgttcagggtgcgctgtgcgagatgcaacagtccggtgtctggctcggcgctctctggtgggccgcaggcccatggtggggcacggtacgtgactgacctcttgctgcgtgtgcacgtgcagactgctcattgctgccacagtatttccaatcgatcgagcccccgagtggcgccgcgatcccctccatcctcccgcaggcactcgaacccttcctgtga

SEQ ID NO: 25 - protein, 386 aa, Phanerochaete chrysosporium
MLKYASIALALATLGVAQQQQWGQCGGIGWTGATTCVAGSVCSVLNPYYSQCIPGAATVTSSSAPSTPTPPAGALPRLGGVNTAGYDFSVATDGSFTGTGVSPPVSQFSHFSSQGANLYRILFAWQLMTPTLGGTISQSFLSRYDQTVQAALNSGPNVFVIIDLHNYARWNGGIIAQGGPTDAQFQSIWTQLAQKYGSNQRVIFGIMNEPHDIPSISTWVNSVQGAVNAIRAAGATNYLLLPGSSWSSAQAFPTEAGPLLVKVTDPLGGTSKLIFDVHKYLDSDNSGTHPDCTTDNVQVLQTLVQFLQANGNRQAILSETGGGNTSSCESLLANELAYVKSAYPTLAGFSVWAAGAFDTTYVLTVTPNADGSDQPLWVDAVKPNLP

SEQ ID NO: 26 - DNA, 20 bp, Artificial Sequence
  Synthetic DNA
atgatacctctccgctctgc

SEQ ID NO: 27 - DNA, 20 bp, Artificial Sequence
  Synthetic DNA
tatcttcctgatgcgattcc

SEQ ID NO: 28 - DNA, 2138 bp, Phanerochaete chrysosporium
atgatacctctccgctctgcagtcgcgtcctcactacttctcgccagtctcggggctgcccagctcccgctgccgaaccctccgtgggtgccgccgaacgcaacgttcgggacgcaccctagcaatccttctgacgggtccgggaacccgcactggacgaatatcctcgggagcacgctctacttctatgaagaacagcggagcggaaagctgcccgttacgaaccgcgtcccgtggcgaaacgatagtgcgacggacgacggcagggacgtcggactggacctgagtggcggctactatgatgctggaggttcgtatttgagtacgctctcccgagaggtcgtctaaccttccggtgtagactacatcaagtatacctttcctatggtgcgtaacacgcagcgtactggttctgaaagctaatgtcaatggcgacccagtccttctctgttatgtctatatgctggggcgcgctggattatggcaagggtaaggcaagaatttctcgttcgctgccctccattgaaacccgtctgcgttaggttatgacctcgcaaaccaaaccgcgtatctcgacgacatgctccggtggagcctagactggctaatgaaggtatggtcaaggatatacttgcatcgacttacctacttgtctgacgatgcatgtcaaggcccatcctgacccaaacacgctgtatgtacaaatcggggatgcagatctggacaacgcatattggggaggcgacagaggcatacctacaccaagaacttcatatgcaatcaacagtaccaggtgcgcgctttgtcgcgttatcctctaggcaatactaagattttgcttagccccggtacagacgccgccgcacaagccgccgcggctttcgctgcctgttctgcattatacaacaatcgaacattgtcgcagcccgcacctaacggtataacaaatacatcttacgcatcgacgctccttcagcatgcgcaacaactgtatcattttgccacaaactcttctatacctcaagttacctaccagacgtccgtgccgtcggtcgccgacgcatacgcatcctccggattccaagacgagctagccatcgcggctctgtttatctcgcttgcaggaaattctagcgacgcttactcgcaagcgtctcagctttaccgcaagcaagggctttctaagcacttggaagatgacgctgtgttcaactgggatgagaagtcacccggcgtcgcccttcttgcggcgcagattgcgcagaagtatcctcagctcgcgaacggcactggtgtggattggaagagcgacctgaacaactacttcgatcgcatcgtcaacaacagtggcagatcattcctaacatcaggtaagcgtgtcaatctcttatcccagttcttcgtgctgacagttcgctctaggcggtctgctttattatccaggcgattcagacgacgctacccttaacccggcattgaacgctgccatgctcctcttgcgctatgccgactcgggctttgcatccagcagtgagaagcagtctgtttaccgccagttcgcccagtctcaaatcgactatttcttgggcaataacccaatgactggtgggtagtgttttcttgacattatgtctatccgcgtcttactcgttgcagtaccgtatatggttggcgtgcacccgaacgcgccatcaaaccctcattctgccttggctacgggtgcttcaccccaggacatcgcgaacatcgacacggtcccagagcacgaggcttacgtcctttatggcggagtagttggagggccgaatgatgacgacctcttttgggacctacggagcgattgggtggagaacgaggttgggctggactacgttgccccggtcgtgaccatcgcggcgcgggaactcgtcagtggagcaggcgacccttggtacacacagctgcaggttgggtcgtacgaggagcggagaccgggtggccagccttgtgatgctgcaatctctgcgggatgtcgcggccatgattggagagtgggcaagatcgtcatgggtgtcttggtcggagtgactggtctcgtggtattgtctcttggtaccgtgtggatggtgttggcatataggaatcgcatcaggaagata

SEQ ID NO: 29 - protein, 590 aa, Phanerochaete chrysosporium
MIPLRSAVASSLLLASLGAAQLPLPNPPWVPLNATFGTHPSNPSDGSGNPHWTNFLENTLYFYEEQRSGKLPVTNRVPWRNDSATDDGRDVGLDLSGGYYDAGDYIKYTFPMSFSVMSICWGALDYGKGYDLANQTAYLDDMLRWSLDWLMKAHPDPNTLYVQVGDADLDNAYWGGDRGIPTPRTSYAINSTSPGTDAAAQAAAAFAACSALYNNRTLSQPAPNGITSTSYASTLLQHAQQLYNFATNSSVPQVTYQASEPSVADAYASSGFQDELAIAALFISLAGNSSDAYPQASQVYRKQGLSKHLEDDAVFNWDEKSPGVALLAAQIAQKYPELANGTGVDWKSDLNNYFDRIVSNSGRSFLTSGGLLYYPGDSDDATLNPALNAAMLLLRYADSGLASSSEKQSAYRQFAQSQIDYFLGNNPMTVQYMVGVHPNAPSNPHSALATGATPQDIANIDTVPEHEAYVLYGGVVGGPNDDDLFWDLRSDWVESEVGLDYVAPVVTIAARELVSGAGDPWYTQLQAGSYEERRPGGQPCDAAISAGCRGHDWRVGKIVMGALVGVTGLVVLSLGTVWMVLAYRNRIRKI

SEQ ID NO: 30 - DNA, 18 bp, Artificial
  Synthetic DNA
catggtgtgtggtggatg

SEQ ID NO: 31 - DNA, 19 bp, Artificial
  Synthetic DNA
aagttcaagagtctcctgt

SEQ ID NO: 32 - DNA, 20 bp, Artificial
  Synthetic DNA
ggtacagtacttatctgtat

SEQ ID NO: 33 - DNA, 30 bp, Artificial
  Synthetic DNA
tctagatttactggtaccccaacaacaatg

SEQ ID NO: 34 - DNA, 31 bp, Artificial
  Synthetic DNA
ccatgggttgatcgacgggttgtcagacacg

SEQ ID NO: 35 - DNA, 17 bp, Artificial
  Synthetic DNA
tctagagtcacctccgt

SEQ ID NO: 36 - DNA, 16 bp, Artificial
  Synthetic DNA
aagcttgggtactgtg

SEQ ID NO: 37 - DNA, 24 bp, Artificial
  Synthetic DNA
tctagagccaacctcgaggggtgg

SEQ ID NO: 38 - DNA, 24 bp, Artificial
  Synthetic DNA
ccatgggaacgtcgagccgatggg

SEQ ID NO: 39 - DNA, 24 bp, Artificial
  Synthetic DNA
tctagagccaacgtcctcggctgg

SEQ ID NO: 40 - DNA, 24 bp, Artificial
  Synthetic DNA
ccatgggtaggtcgagccgatggg

SEQ ID NO: 41 - DNA, 24 bp, Artificial
  Synthetic DNA
tctagagccaacgtcctcggctgg

SEQ ID NO: 42 - DNA, 25 bp, Artificial
  Synthetic DNA
ccatggagcgtaggtcgagccaatg

SEQ ID NO: 43 - DNA, 24 bp, Artificial
  Synthetic DNA
tctagaatctacctgagcccttac

SEQ ID NO: 44 - DNA, 24 bp, Artificial
  Synthetic DNA
ccatggctcactagtggcgagacc

SEQ ID NO: 45 - DNA, 25 bp, Artificial
  Synthetic DNA
tctagagctcacggtttcattcatg

SEQ ID NO: 46 - DNA, 25 bp, Artificial
  Synthetic DNA
ccatggggtgtagagccccggaatg

SEQ ID NO: 47 - DNA, 24 bp, Artificial
  Synthetic DNA
tctagagcgggcccgtactcgctc

SEQ ID NO: 48 - DNA, 24 bp, Artificial
  Synthetic DNA
ccatgggtaatgtgattcctgtcg

SEQ ID NO: 49 - DNA, 24 bp, Artificial
  Synthetic DNA
tctagaatgaagtacttcttgctc

SEQ ID NO: 50 - DNA, 24 bp, Artificial
  Synthetic DNA
ccatggcgtttggcgtaccgtctg

SEQ ID NO: 51 - DNA, 24 bp, Artificial
  Synthetic DNA
tctagaccccggtacagacgccgc

SEQ ID NO: 52 - DNA, 24 bp, Artificial
  Synthetic DNA
ccatgggatgttaggaatgatctg

(19) United States
(12) Patent Application Publication
(10) Pub. No.: US 2005/0181485 A1
(43) Pub. Date: Aug. 18, 2005

(54) Cellulose digesting enzyme gene and utilization of the gene

(76) Inventors: Akira Tsukamoto, Tokyo (JP); Seiji Nakagame, Chiba (JP); Mari Kabuto, Chiba (JP); Jun Sugiura, Kanagawa (JP); Hisako Sakaguchi, Chiba (JP); Atsushi Furujyo, Mie (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/505,474

(22) PCT Filed: Feb. 25, 2003
(86) PCT No.: PCT/JP03/02058

(30) Foreign Application Priority Data
Feb. 25, 2002 (JP) 2002-48675

(51) Int. Cl. D21C 1/00; C12N 9/42; C12P 19/04; C12N 1/14; C12N 15/74

(52) U.S. Cl. 435/101; 435/209; 435/254.1; 435/484; 162/70

(57) ABSTRACT

A method for treating woodchips, comprising the steps of: preparing a DNA encoding an antisense RNA substantially complementary to the whole or a part of a transcription product of a cellulolytic enzyme gene derived from Basidiomycete; preparing a vector comprising (a) the above DNA, or (b) a recombinant DNA comprising the above DNA and a DNA fragment having a promoter activity, wherein the above DNA binds to the above DNA fragment such that an antisense RNA of the cellulolytic enzyme gene is generated as a result of transcription; transforming host cells with the above vector, so as to prepare the host cells having a suppressed cellulolytic enzyme activity; and inoculating the above host cells having a suppressed cellulolytic enzyme activity into woodchips to treat them.

### TECHNICAL FIELD

The present invention relates to a method for treating woodchips using an antisense gene of a gene encoding cellulolytic enzyme.

### BACKGROUND ART

Pulp produced in the paper and pulp industry is divided into mechanical pulp and chemical pulp in terms of a production method thereof.

Mechanical pulp is produced by physically grinding wood fibers with mechanical energy. Since such mechanical pulp contains almost all wood components, it can be produced at a high yield, and thereby, a thin paper with high opacity can be produced. However, mechanical pulp has disadvantages that a high electric power is required for grinding and that the produced paper hardly has paper strength.

Microorganisms, which solve the above problems and do not decrease pulp yield, have widely been screened. For example, there has been a report that when alder first refined thermomechanical pulp (TMP) was treated with a Basidiomycete, Phanerochaete chrysosporium, in the presence of glucose, and it was then subjected to the second refining, energy necessary for refining was reduced by 25% to 30% (Bar-Lev and T. K. Kirk, Tappi J., 65, 111, 1982). Moreover, when aspen lumbers were treated with Phanerochaete chrysosporium and Dichomitus squalens, the obtained paper had paper strength higher than that of a control (Myers., Tappi J., 105, 1988). Akamathu et al. have cultured 10 strains of white-rot fungi including Coriolus hirsutus on poplar lumbers, so as to examine pulp yield, refining energy, and pulp strength. As a result, they have found that, among the used strains, Coriolus hirsutus was preferable as a fungus used in pretreatment of chips because this fungus caused decrease in refining energy and increase in the degree of crystallinity. However, at the same time, Coriolus hirsutus caused a yield of about 7% (Akamathu et al., Mokuzai gakkaishi, 30 (8) 697-702, 1984). Moreover, using 10 types of white-rot fungi preliminarily selected from 61 types (85 strains) of white-rot fungi, Nishibe et al. have microbially decomposed secondary refined TMP from Pterocarya rhoifolia wood pieces and softwood. Thereafter, they have selectively performed delignification. They have selected Coprinus cinereus and Phanerochaete chrysosporium, which caused less degradation of pulp fibers, and they have shown that these fungi could control reduction in paper strength in the presence of glucose and urea. However, when pulp was treated with the above fungi at 30° C. for 14 days, decreases in pulp yield were 6.3% and 9.7%, respectively. When Coriolus hirsutus was used, decrease in yield was 7.5% (Nishibe et al., Japan Tappi, 42 (2), 1988). Kashino et al. have screened a white-rot fungus IZU-154 from the nature, and lignin has been selectively decomposed with Phanerochaete chrysosporium or Trametes versicolor. They have confirmed that when a hardwood tree was thus treated for 7 days, refining energy was decreased by ½ to ⅔. In addition, they have also confirmed that pulp strength was approximately two times increased. When a softwood was treated for 10 to 14 days, refining energy was decreased by ⅓, and increase in pulp strength was also observed. When a medium was added, the same results could be obtained for 7 days (Kashino et al., Tappi J., 76 (12), 167, 1993). Furthermore, recently, a lignin consortium consisting of research institutes and several pulp and paper companies, including USDA Forest Products Laboratory as a center, has been established in the U.S.A. The lignin consortium has screened a strain, which has a high ability to decompose lignin but a low ability to decompose cellulose, and as a result, the consortium has newly isolated Ceriporiopsis subvermispora from the nature. The consortium has studied reduction in a power used for mechanical pulp, using this strain. The consotium has reported that this strain enabled reduction in nearly 40% of the energy necessary for producing TMP, for example, and that decrease in yield was approximately 3% to 5% in this case. The consortium has also reported that there were no feared adverse effects on paper strength, but that such strength rather increased. The USDA Forest Products Laboratory has already constructed a pilot plant, and verification tests of the isolated Ceriporiopsis subvernispora are carried out therein. Assuming the U.S. plants, it is also considered that a treatment with microorganisms is carried out in a chip yard in a plant (Scott et al., Tappi J., 81. 12. 153, 1998). In such a case, since the inside of a pile conserving chips has a high temperature, strains having effects even at a high temperature are required. However, since the isolated Ceriporiopsis subvermispora has effects only at a temperature of 32° C. or lower, this fungus is inadequate for practical use.

In addition, microorganisms that have been obtained by the previous screening do not necessarily have high selectivity with respect to lignin decomposition. Since they decompose not only lignin but also cellulose, they result in decrease in pulp yield or paper strength. Thus, it is further desired to obtain or produce microorganisms having enhanced selectivity with respect to lignin decomposition, that is, microorganisms having a suppressed ability to decompose cellulose as well as having an excellent ability to decompose lignin.

Ander et al. have produced a mutant having enhanced selectivity with respect to lignin decomposition. They have introduced a mutation in Sporotrichum pulverulentum by UV radiation, so that they have developed a strain Cel44 having low cellulase activity. Birch wood pieces were decomposed by both a wild type strain and the above cellulase-deficient Cel44. As a result, it was found that the former decomposed well lignin and xylan, whereas the latter decomposed well lignin and xylan but hardly decomposed glucan (Ander and Eriksson, Svensk Papperstidning, 18, 643, 1975). Birch lumbers were treated with this Cel44 for 6 weeks, and thereafter, mechanical pulp was produced therefrom. As a result, it was found that paper strength was increased (Ander and Eriksson, Svensk Papperetidning, 18, 641, 1975). Moreover, there has been a report that when an experiment was carried out using birch lumbers and pine lumbers, energy necessary for fibrillation and refining of fibers was 30% reduced by increasing treating time (Eriksson and Vallander., Svensk Papperstid, 85, R33, 1982). They have also produced Cel26 with a low cellulase activity from Phlebia radiata. Chips and pulp made from pine lumbers were treated with this Cel26, and mechanical pulp was then produced therefrom. As a result, it was found that paper strength was not improved in both cases but that decrease in refining energy was observed. In addition, reduction in the weight was 2% or less (Samuelsson et al., Svensk Papperstid, 8, 221, 1980).

As stated above, mutant strains with a low cellulose activity have been produced, and the use of such mutant strains in a treatment of mechanical pulp has been considered. However, since these mutant strains are mutagenetically treated by ultraviolet radiation, they have problems that their growth rate is slow and it takes a long time to decompose pulp. Accordingly, it is desired to produce a mutant strain, which has a normal growth rate, but has only its cellulolytic activity of which is suppressed.

On the other hand, chemical pulp is obtained by a production method comprising solving lignin from lumbers using chemicals, so as to obtain cellulose and hemicellulose. At present, Kraft pulp involving delignification with sodium hydroxide and sodium sulfate has become mainstream. As with mechanical pulp, Kraft pulp is also treated with microorganisms and subjected to delignification before cooking, whereby reduction of production energy and improvement of quality of pulp are attempted.

For example, there has been a report that when red oak lumbers or aspen lumbers are treated with Phanerochaete chrysosporium for 30 days, yield is improved at the same Ka value and beating energy is thereby reduced, and tensile strength and bursting strength are increased (Oriaran et al., Tappi, 73, 147, 1990). Likewise, increase in bursting strength and tearing strength has been reported from an experiment using Phanerochaete chrysosporium (Chen et al., Wood Fiber Sci., 27. 198, 1995). Molina et al. have reported that when radiata pine was treated with Trametes versicolor and Pleurotus ostreatus, production energy could be 11% to 14% reduced. In the case of Trametes versicolor, however, decrease in pulp strength was observed (Molina, 50th Appita Annual General Conference, pp. 57-63, 1996; Molina, 51st Annual General Conference, pp. 199-206). Bajpai et al. have reported from an experiment using Ceriporiopsis subvermispora that using the microorganism, active alkali could be reduced by 18%, cooking time could be reduced by 33%, and sulfur degree in white liquor could be reduced by 30% (P. Bajpai et al. J. Pulp and Paper Science: 27 (7), 235-239, 2001).

As stated above, a treatment of Kraft pulp with microorganisms improves cooking, thereby resulting in reduction in energy. However, in some cases, such a treatment with microorganisms might decrease yield or paper strength. Accordingly, as in the case of mechanical pulp, for the practical use of a treatment with microorganisms, achievement or production of microorganisms with enhanced selectivity with respect to lignin decomposition is required, which has an excellent ability to decompose lignin but has a suppressed ability to decompose cellulose.

Among such cellulolytic enzymes (cellulose-decomposing enzymes), an enzyme generically called cellulase hydrolyzes the bond of β-1,4-glucan (cellulose) or a derivative thereof to β-1,4-glucopyranosyl. This enzyme is widely distributed in higher plants, microorganisms such as fungi or bacteria, mollusks, etc. It has been known that cellulase is broadly divided into what is called endoglucanase (CMCase) hydrolyzing the β-1,4-glucopyranosyl bond of a cellulose main chain in an endo-manner, and into what is called exoglucanase (avicelase) eliminating mainly cellobiose residues from the terminus of a cellulose main chain. These hydrolytic enzymes synergistically act on cellulose, so that the cellulose substrate reduces its molecular weight to generate cellobiose, and further, due to involvement of β-glucosidase, it is decomposed into glucose units.

Moreover, cellobiose dehydrogenase is oxidoreductase, which oxidizes cellobiose or cellooligosaccharide to generate cellobionolactone and, at the same time, also reduces quinone, metal complexes of such as iron, phenoxy radical, or oxygen. This enzyme is generated at the same time with cellulase when microorganisms decompose cellulose (Eriksson et al., FEBS Lett., 49, 282-285, 1974). In addition, this enzyme releases the inhibition of cellulase activity due to cellobiose, that is, inhibition of products due to cellobiose (Igarashi et al., Eur. J. Biochem., 253, 101, 1998). From these facts, it is considered that cellobiose dehydrogenase is conjugated with cellulase to promote decomposition of cellulose. Furthermore, since cellobiose dehydrogenase causes a Fenton reaction, which generates a hydroxyl radical strongly decomposing cellulose, it is considered that this enzyme is highly associated with decomposition of cellulose. This inference is supported also from the production of mutant strains having suppressed cellobiose dehydrogenase activity by Dumonceaux et al., and analysis results of the properties thereof. Dumonceaux et al. have produced a cellobiose dehydrogenase-deficient strain by homologous recombination, using an antibiotic ferromycin as an index. They have reported that the growth rate of the cellobiose dehydrogenase-deficient strain is almost the same as that of a wild type stain when amorphous cellulose is used as a carbon source, but that the growth rate of the cellobiose dehydrogenase-deficient strain becomes significantly slow when it is cultured on crystalline cellulose, and that the cellobiose dehydrogenase-deficient strain decomposes lignin contained in unbleached pulp from broad-leaved trees or synthetic lignin ¹⁴C-DHP at a level equivalent to a wild type strain (Dumonceaux, Enzyme and Microb. 29, 478-489, 2001).

According to the general introduction regarding cellobiose dehydrogenase or the like (G. Henriksson et al., J. Biotechnol. 78 (2000) 93-113), examples of microorganisms producing this enzyme may include wood rotting fungi such as Phanerochaete chrysosporium, Trametes versicolor, Schizophyllum commune, Coneophora puteana, Myceliophtore thermophila, or Fumicola insolens.

In addition, with regard to a gene encoding cellobiose dehydrogenase (hereinafter referred to as a cellobiose dehydrogenase gene), in the case of Phanerochaete chrysosporium for example, cDNA of the K3 strain (Raices et al., FEBS Letters, 69, 233-238, 1995), and the cDNA (Li et al., Appl. Environ. Microbiol., 62(4), 1329-1335, 1996) and chromosomal DNA of the OGC101 strain have been cloned (Li et al. Appl. Environ. Microbiol., 63(2), 796-799, 1997). Moreover, with regard to Trametes versicolor (T. J. Dumonceaux et al., Gene. 210. 211-219 (1998)) and Pycnoporus cinnabarinus (S. M. Moukha et al., Gene, 234, 23-33, 1999) also, the presence of cellobiose dehydrogenase genes has been reported.

As stated above, cellobiose dehydrogenase and genes encoding the enzyme have been disclosed. However, there have been no reports regarding clarification of cellulolytic enzymes including cellobiose dehydrogenase derived from Coriolus hirsutus as a typical example, or cellulolytic enzyme genes, that are necessary for achievement or production of microorganisms with enhanced selectivity with respect to lignin decomposition, used in a treatment with microorganisms in the production of mechanical pulp or chemical pulp, and regarding genetic recombination techniques of applying the above genes. Further, no effective methods for treating pulp using a transformant obtained by such genetic recombination have been disclosed.

On the other hand, use of cellulolytic enzymes has attracted keen interest for a long time. For example, use of cellulolytic enzymes in various ways, such as addition of the enzymes into household detergents, reforming of cellulose polymeric materials such as fibers due to surface treatment with the enzymes, deinking treatment from waste papers, or food processing, has been studied. Thus, a method for producing a large quantity of cellulolytic enzyme has been required. As an attempt to produce a large quantity of cellobiose dehydrogenase, there has been a report that D-glycerylaldehyde-3-phosphate dehydrogenase as a structurally functioning promoter was ligated upstream of the cellobiose dehydrogenase-1 gene of Phanerochaete chrysosporium (Li., Biochem. Biophys. Res. Commun., 270, 141-146, 2000). However, since Phanerochaete chrysosporium is designated as a destructive fungus in Japan, it cannot be used. Hence, it is desired to develop a technique of producing a large quantity of cellulolytic enzymes including cellobiose dehydrogenase as a typical example, using harmless microorganisms, but so far there have been no reports regarding the above techniques of using cellulolytic enzyme genes such as a cellobiose dehydrogenase gene derived from Coriolus hirsutus.

### DISCLOSURE OF THE INVENTION

It is an object of the present invention to provide a method for treating woodchips using an antisense gene of a gene encoding cellulolytic enzyme.

In order to solve the above-described problems, the present inventors have intensively studied and widely screened for fungi producing cellulolytic enzymes including cellobiose dehydrogenase as a typical example. As a result, they have found that Coriolus hirsutus produces cellulolytic enzymes. Moreover, the present inventors have also succeeded in cloning genes encoding such cellulolytic enzymes. Furthermore, they have developed a method for controlling the expression of the above gene using an antisense gene thereof. They have produced pulp from woodchips according to the above method and have succeeded in controlling yield or reduction in paper strength, thereby completing the present invention.

That is to say, the present invention provide the following features:
(1) A method for treating woodchips, comprising the steps of:
   preparing a DNA encoding an antisense RNA substantially complementary to the whole or a part of a transcription product of a cellulolytic enzyme gene derived from Basidiomycete;
   preparing a vector comprising (a) the above DNA, or (b) a recombinant DNA comprising the above DNA and a DNA fragment having a promoter activity, wherein the above DNA binds to the above DNA fragment such that an antisense RNA of the cellulolytic enzyme gene is generated as a result of transcription;
   transforming host cells with the above vector, so as to prepare the host cells having a suppressed cellulolytic enzyme activity; and
   inoculating the above host cells having a suppressed cellulolytic enzyme activity into woodchips to treat them.
(2) The method according to (1), wherein the cellulolytic enzyme gene comprises one or more genes selected from the group consisting of respective genes encoding a cellobiose dehydrogenase, cellobiohydrolase I, cellobiohydrolase II, an endoglucanase belonging to glycolytic enzyme family 61, an endoglucanase belonging to glycolytic enzyme family 12, an endoglucanase belonging to glycolytic enzyme family 5, and an endoglucanase belonging to glycolytic enzyme family 9.
(3) The method according to (2), wherein the cellobiose dehydrogenase gene is an isolated cellobiose dehydrogenase gene comprising any one of the following nucleotide sequences (a) to (c):
   (a) a nucleotide sequence as shown in SEQ ID No. 1 or 3;
   (b) a nucleotide sequence hybridizing with a nucleotide sequence comprising a nucleotide sequence complementary to the nucleotide sequence according to (a) under stringent conditions, and encoding a protein having a cellobiose dehydrogenase enzyme activity; and
   (c) a nucleotide sequence comprising deletion, substitution or addition of one or more nucleotides with respect to SEQ ID No. 1 or 3, and encoding a protein having a cellobiose dehydrogenase enzyme activity.
(4) The method according to (2), wherein the cellobiohydrolase I gene is an isolated cellobiohydrolase I gene comprising any one of the following nucleotide sequences (a) to (c):
   (a) a nucleotide sequence as shown in SEQ ID No. 7, 9, or 11;
   (b) a nucleotide sequence hybridizing with a nucleotide sequence comprising a nucleotide sequence complementary to the nucleotide sequence according to (a) under stringent conditions, and encoding a protein having a cellobiohydrolase I gene enzyme activity; and
   (c) a nucleotide sequence comprising deletion, substitution or addition of one or more nucleotides with respect to SEQ ID No. 7, 9 or 11, and encoding a protein having a cellobiohydrolase I gene enzyme activity.
(5) The method according to (2), wherein the cellobiohydrolase II gene is an isolated cellobiohydrolase II gene comprising any one of the following nucleotide sequences (a) to (c):
   (a) a nucleotide sequence as shown in SEQ ID No. 14;
   (b) a nucleotide sequence hybridizing with a nucleotide sequence comprising a nucleotide sequence complementary to the nucleotide sequence according to (a) under stringent conditions, and encoding a protein having a cellobiohydrolase II gene enzyme activity; and
   (c) a nucleotide sequence comprising deletion, substitution or addition of one or more nucleotides with respect to SEQ ID No. 14, and encoding a protein having a cellobiohydrolase II gene enzyme activity.
(6) The method according to (2), wherein the endoglucanase gene belonging to glycolytic enzyme family 61 is an isolated endoglucanase gene belonging to glycolytic enzyme family 61 comprising any one of the following nucleotide sequences (a) to (c):
   (a) a nucleotide sequence as shown in SEQ ID No. 18;
   (b) a nucleotide sequence hybridizing with a nucleotide sequence comprising a nucleotide sequence complementary to the nucleotide sequence according to (a) under stringent conditions, and encoding a protein having an activity of endoglucanase enzyme belonging to glycolytic enzyme family 61; and
   (c) a nucleotide sequence comprising deletion, substitution or addition of one or more nucleotides with respect to SEQ ID No. 18, and encoding a protein having an activity of endoglucanase enzyme belonging to glycolytic enzyme family 61.
(7) The method according to (2), wherein the endoglucanase gene belonging to glycolytic enzyme family 12 is an isolated endoglucanase gene belonging to glycolytic enzyme family 12 comprising any one of the following nucleotide sequences (a) to (c):
   (a) a nucleotide sequence as shown in SEQ ID No. 20;
   (b) a nucleotide sequence hybridizing with a nucleotide sequence comprising a nucleotide sequence complementary to the nucleotide sequence according to (a) under stringent conditions, and encoding a protein having an activity of endoglucanase enzyme belonging to glycolytic enzyme family 12; and
   (c) a nucleotide sequence comprising deletion, substitution or addition of one or more nucleotides with respect to SEQ ID No. 20, and encoding a protein having an activity of endoglucanase enzyme belonging to glycolytic enzyme family 12.
(8) The method according to (2), wherein the endoglucanase gene belonging to glycolytic enzyme family 5 is an isolated endoglucanase gene belonging to glycolytic enzyme family 5 comprising any one of the following nucleotide sequences (a) to (c):
   (a) a nucleotide sequence as shown in SEQ ID No. 24;
   (b) a nucleotide sequence hybridizing with a nucleotide sequence comprising a nucleotide sequence complementary to the nucleotide sequence according to (a) under stringent conditions, and encoding a protein having an activity of endoglucanase enzyme belonging to glycolytic enzyme family 5; and
   (c) a nucleotide sequence comprising deletion, substitution or addition of one or more nucleotides with respect to SEQ ID No. 24, and encoding a protein having an activity of endoglucanase enzyme belonging to glycolytic enzyme family 5.
(9) The method according to (2), wherein the endoglucanase gene belonging to glycolytic enzyme family 9 is an isolated endoglucanase gene belonging to glycolytic enzyme family 9 comprising any one of the following nucleotide sequences (a) to (c):
   (a) a nucleotide sequence as shown in SEQ ID No. 28;
   (b) a nucleotide sequence hybridizing with a nucleotide sequence comprising a nucleotide sequence complementary to the nucleotide sequence according to (a) under stringent conditions, and encoding a protein having an activity of endoglucanase enzyme belonging to glycolytic enzyme family 9; and
   (c) a nucleotide sequence comprising deletion, substitution or addition of one or more nucleotides with respect to SEQ ID No. 28, and encoding a protein having an activity of endoglucanase enzyme belonging to glycolytic enzyme family 9.
(10) The method according to any one of (1) to (9), wherein Basidiomycete is Coriolus hirsutus or Phanerochaete chrysosporium.
(11) The method according to any one of (1) to (10), wherein host cells are Coriolus hirsutus.
(12) A woodchip obtained by the method according to any one of (1) to (11).
(13) A method for producing a pulp using the woodchip according to (12).
(14) A pulp obtained by the method according to (13).

This specification includes part or all of the contents as disclosed in the specification and/or drawings of Japanese Patent Application No. 2002-48675, which is a priority document of the present application.

### BRIEF DESCRIPTION OF THE DRAWINGS

FIG. 1 is a view showing analysis results of a reaction of acting the cellobiose dehydrogenase of the present invention on cellobiose;

FIG. 2 is a view showing optimal pH of the cellobiose dehydrogenase of the present invention. In the figure, ♦ represents glycine-HCl, ▪ represents acetic acid, and ▴ represents phosphoric acid;

FIG. 3 is a view showing pH stability of the cellobiose dehydrogenase of the present invention. In the figure, ♦represents glycine-HCl, ▪ represents acetic acid, and ▴ represents phosphoric acid;

FIG. 4 is a view showing an optimal reaction temperature of the cellobiose dehydrogenase of the present invention; and

FIG. 5 is a view showing heat stability of the cellobiose dehydrogenase of the present invention.

### DESCRIPTION OF SEQUENCE LISTING

SEQ ID No. 5 is a probe used in plaque hybridization.

SEQ ID No. 6 is a probe used in plaque hybridization.

SEQ ID No. 13 is a probe used in plaque hybridization.

SEQ ID No. 16 is a primer used in a PCR reaction.

SEQ ID No. 17 is a primer used in a PCR reaction.

SEQ ID No. 22 is a primer used in a PCR reaction.

SEQ ID No. 23 is a primer used in a PCR reaction.

SEQ ID No. 26 is a primer used in a PCR reaction.

SEQ ID No. 27 is a primer used in a PCR reaction.

SEQ ID No. 30 is a primer used in a PCR reaction.

SEQ ID No. 31 is a primer used in a PCR reaction.

SEQ ID No. 32 is a primer used in a PCR reaction.

SEQ ID No. 33 is a primer used in a PCR reaction.

SEQ ID No. 34 is a primer used in a PCR reaction.

SEQ ID No. 35 is a primer used in a PCR reaction.

SEQ ID No. 36 is a primer used in a PCR reaction.

SEQ ID No. 37 is a primer used in a PCR reaction.

SEQ ID No. 38 is a primer used in a PCR reaction.

SEQ ID No. 39 is a primer used in a PCR reaction.

SEQ ID No. 40 is a primer used in a PCR reaction.

SEQ ID No. 41 is a primer used in a PCR reaction.

SEQ ID No. 42 is a primer used in a PCR reaction.

SEQ ID No. 43 is a primer used in a PCR reaction.

SEQ ID No. 44 is a primer used in a PCR reaction.

SEQ ID No. 45 is a primer used in a PCR reaction.

SEQ ID No. 46 is a primer used in a PCR reaction.

SEQ ID No. 47 is a primer used in a PCR reaction.

SEQ ID No. 48 is a primer used in a PCR reaction.

SEQ ID No. 49 is a primer used in a PCR reaction.

SEQ ID No. 50 is a primer used in a PCR reaction.

SEQ ID No. 51 is a primer used in a PCR reaction.

SEQ ID No. 52 is a primer used in a PCR reaction.

### MODE FOR CARRYING OUT THE INVENTION

The present invention will be described further in detail below.

The present invention provides a method for treating woodchips, which comprises the steps of: preparing DNA encoding antisense RNA substantially complementary to the whole or a part of a transcription product of a cellulolytic enzyme gene derived from Basidiomycete; preparing a vector comprising (a) the above DNA, or (b) recombinant DNA comprising the above DNA and a DNA fragment having a promoter activity, wherein the above DNA binds to the above DNA fragment such that antisense RNA of a cellulolytic enzyme gene is generated as a result of transcription; performing transformation with the above vector, so as to prepare host cells having a suppressed cellulolytic enzyme activity; and inoculating the above host cells having a suppressed cellulolytic enzyme activity into woodchips, thereby treating them.

In the present specification, any type of Basidiomycete can be used, as long as it has an ability to decompose cellulose. In particular, Coriolus hirsutus whose Japanese name is Aragekawaratake is preferable.

Moreover, a cellulolytic enzyme gene is not particularly limited, as long as the enzyme can decompose cellulose. Preferred examples of such a cellulolytic enzyme gene may include a cellobiose dehydrogenase gene, a cellobiohydrolase I gene, a cellobiohydrolase II gene, and an endoglucanase gene. More specifically, various genes described in 1 to 7 below can be used. It is to be noted that these genes can be used singly, but they may be used in combination of one or more types.
1. An isolated cellobiose dehydrogenase gene comprising any one of the following nucleotide sequences (a) to (c):
   (a) a nucleotide sequence as shown in SEQ ID No. 1 or 3;
   (b) a nucleotide sequence hybridizing with a nucleotide sequence having a nucleotide sequence complementary to the nucleotide sequence according to (a) under stringent conditions, and encoding a protein having a cellobiose dehydrogenase enzyme activity; and
   (c) a nucleotide sequence comprising deletion, substitution or addition of one or more nucleotides with respect to SEQ ID No. 1 or 3, and encoding a protein having a cellobiose dehydrogenase enzyme activity.
2. An isolated cellobiohydrolase I gene comprising any one of the following nucleotide sequences (a) to (c):
   (a) a nucleotide sequence as shown in SEQ ID No. 7, 9, or 11;
   (b) a nucleotide sequence hybridizing with a nucleotide sequence having a nucleotide sequence complementary to the nucleotide sequence according to (a) under stringent conditions, and encoding a protein having a cellobiohydrolase I gene enzyme activity; and
   (c) a nucleotide sequence comprising deletion, substitution or addition of one or more nucleotides with respect to SEQ ID No. 7, 9 or 11, and encoding a protein having a cellobiohydrolase I gene enzyme activity.
3. An isolated cellobiohydrolase II gene comprising any one of the following nucleotide sequences (a) to (c):
   (a) a nucleotide sequence as shown in SEQ ID No. 14;
   (b) a nucleotide sequence hybridizing with a nucleotide sequence having a nucleotide sequence complementary to the nucleotide sequence according to (a) under stringent conditions, and encoding a protein having a cellobiohydrolase II gene enzyme activity; and
   (c) a nucleotide sequence comprising deletion, substitution or addition of one or more nucleotides with respect to SEQ ID No. 14, and encoding a protein having a cellobiohydrolase II gene enzyme activity.
4. An isolated endoglucanase gene belonging to glycolytic enzyme family 61 comprising any one of the following nucleotide sequences (a) to (c):
   (a) a nucleotide sequence as shown in SEQ ID No. 18;
   (b) a nucleotide sequence hybridizing with a nucleotide sequence having a nucleotide sequence complementary to the nucleotide sequence according to (a) under stringent conditions, and encoding a protein having an activity of endoglucanase enzyme belonging to glycolytic enzyme family 61; and
   (c) a nucleotide sequence comprising deletion, substitution or addition of one or more nucleotides with respect to SEQ ID No. 18, and encoding a protein having an activity of endoglucanase enzyme belonging enzyme to glycolytic enzyme family 61.
5. An isolated endoglucanase gene belonging to glycolytic enzyme family 12 comprising any one of the following nucleotide sequences (a) to (c):
   (a) a nucleotide sequence as shown in SEQ ID No. 20;
   (b) a nucleotide sequence hybridizing with a nucleotide sequence having a nucleotide sequence complementary to the nucleotide sequence according to (a) under stringent conditions, and encoding a protein having an activity of endoglucanase enzyme belonging to glycolytic enzyme family 12; and
   (c) a nucleotide sequence comprising deletion, substitution or addition of one or more nucleotides with respect to SEQ ID No. 20, and encoding a protein having an activity of endoglucanase enzyme belonging to glycolytic enzyme family 12.
6. An isolated endoglucanase gene belonging to glycolytic enzyme family 5 comprising any one of the following nucleotide sequences (a) to (c):
   (a) a nucleotide sequence as shown in SEQ ID No. 24;
   (b) a nucleotide sequence hybridizing with a nucleotide sequence having a nucleotide sequence complementary to the nucleotide sequence according to (a) under stringent conditions, and encoding a protein having an activity of endoglucanase enzyme belonging to glycolytic enzyme family 5; and
   (c) a nucleotide sequence comprising deletion, substitution or addition of one or more nucleotides with respect to SEQ ID No. 24, and encoding a protein having an activity of endoglucanase enzyme belonging to glycolytic enzyme family 5.
7. An isolated endoglucanase gene belonging to glycolytic enzyme family 9 comprising any one of the following nucleotide sequences (a) to (c):
   (a) a nucleotide sequence as shown in SEQ ID No. 28;
   (b) a nucleotide sequence hybridizing with a nucleotide sequence having a nucleotide sequence complementary to the nucleotide sequence according to (a) under stringent conditions, and encoding a protein having an activity of endoglucanase enzyme belonging to glycolytic enzyme family 9; and
   (c) a nucleotide sequence comprising deletion, substitution or addition of one or more nucleotides with respect to SEQ ID No. 28, and encoding a protein having an activity of endoglucanase enzyme belonging to glycolytic enzyme family 9.

The cellobiose dehydrogenase gene described in 1. above can be obtained by the following procedure.

Chromosomal DNA is prepared from Coriolus hirsutus by a common method of extracting chromosomal DNA, such as the method of Yelton et al. (Proc. Natl. Acad. Sci. USA, 81, 1470 (1984)). Subsequently, the obtained chromosomal DNA is treated with suitable restriction enzymes such as Sau3AI for partial decomposition, and the resultant product is fractionated by sucrose density gradient ultracentrifugation, so as to obtain DNA fragments with a size from 10 kbp to 25 kbp. The thus obtained DNA fragment is ligated to phage DNA, which has been treated with restriction enzymes generating the same cohesive termini. EMBL3 (A-M, Frishauf et al., J. Mol. Biol. 170, 827 (1983)) λ phage DNA is an example of such phage DNA. The obtained DNA fragment-ligated phage is subjected to in vitro packaging, and the resultant product is used as a chromosomal DNA library. For subcloning, a commonly used cloning vector, and preferably an Escherichia coli vector can be used. For example, a pUC plasmid such as pUC18 (C. Yanisch-Perron, et al., Gene, 33, 103 (1985)) can be used. A cloning vector is not limited to the above example, but commercially available cloning vector or known vectors described in publications can also be used.

In order to isolate a cellobiose dehydrogenase gene from the above-described chromosomal gene library, cellobiose dehydrogenase which has been obtained from Coriolus hirsutus and purified, is completely digested with lysyl endopeptidase, and the digest is then subjected to amino acid sequencing. Thereafter, using synthetic DNA probes produced based on a nucleotide sequence estimated from the obtained amino acid sequence, plaque hybridization is carried out, so as to select clones containing cellobiose dehydrogenase genes. A DNA fragment containing a cellobiose dehydrogenase gene is isolated from the selected clones. Thereafter, a restriction map thereof is prepared, and a sequence thereof is determined. The sequence can be determined by inserting the above fragment containing a cellobiose dehydrogenase gene into a suitable cloning vector (e.g., a pUC vector such as pUC19), and then applying the method of Sanger et al. (Proc. Natl. Acad. Sci. USA, 74, 5463 (1977)).

According to the above-described procedure, nucleotide sequences shown in SEQ ID NOS: 1 and 3 encoding cellobiose dehydrogenase derived from Coriolus hirsutus have been determined. A gene having a 3,420 bp nucleotide sequence as shown in SEQ ID No. 1 is a structural gene of a 7,207 bp cellobiose dehydrogenase genomic gene derived from Coriolus hirsutus, which was named as cellobiose dehydrogenase 1 gene. A gene having a 3,480 bp nucleotide sequence as shown in SEQ ID No. 3 is a structural gene of a 5,345 bp cellobiose dehydrogenase genomic gene derived from Coriolus hirsutus, which was named as cellobiose dehydrogenase 2 gene.

A structural gene portion of the cellobiose dehydrogenase 1 gene shown in SEQ ID No. 1 consists of 16 exons and 15 introns (intervening sequence).

More specifically, exon 1 is located between 129 and 177, intron 1 is located between 178 and 239, exon 2 is located between 240 and 498, intron 2 is located between 499 and 557, exon 3 is located between 558 and 667, intron 3 is located between 668 and 716, exon 4 is located between 717 and 833, intron 4 is located between 834 and 885, exon 5 is located between 886 and 1028, intron 5 is located between 1029 and 1077, exon 6 is located between 1078 and 1242, intron 6 is located between 1243 and 1301, exon 7 is located between 1302 and 1374, intron 7 is located between 1375 and 1425, exon 8 is located between 1426 and 1480, intron 8 is located between 1481 and 1534, exon 9 is located between 1535 and 2165, intron 9 is located between 2166 and 2223, exon 10 is located between 2224 and 2351, intron 10 is located between 2352 and 2407, exon 11 is located between 2408 and 2456, intron 11 is located between 2457 and 2509, exon 12 is located between 2510 and 2598, intron 12 is located between 2599 and 2653, exon 13 is located between 2654 and 2799, intron 13 is located between 2800 and 2859, exon 14 is located between 2860 and 2930, intron 14 is located between 2931 and 2995, exon 15 is located between 2996 and 3100, intron 15 is located between 3101 and 3157, and exon 16 is located between 3158 and 3274. In addition, a region represented by nucleotide No. 3275 and forward is a 3′-nontranslation region including a terminator.

Moreover, it was found that an amino acid sequence estimated from analysis of the nucleotide sequence is an amino acid sequence consisting of 768 amino acid residues, which is shown in SEQ ID No. 2.

On the other hand, a structural gene portion of the cellobiose dehydrogenase 2 gene shown in SEQ ID No. 3 consists of 16 exons and 15 introns.

More specifically, exon 1 is located between 159 and 207, intron 1 is located between 208 and 269, exon 2 is located between 270 and 528, intron 2 is located between 529 and 587, exon 3 is located between 588 and 697, intron 3 is located between 698 and 746, exon 4 is located between 747 and 863, intron 4 is located between 864 and 915, exon 5 is located between 916 and 1058, intron 5 is located between 1059 and 1107, exon 6 is located between 1108 and 1272, intron 6 is located between 1273 and 1331, exon 7 is located between 1332 and 1404, intron 7 is located between 1405 and 1455, exon 8 is located between 1456 and 1510, intron 8 is located between 1511 and 1564, exon 9 is located between 1565 and 2195, intron 9 is located between 2196 and 2253, exon 10 is located between 2254 and 2381, intron 10 is located between 2382 and 2437, exon 11 is located between 2438 and 2486, intron 11 is located between 2487 and 2539, exon 12 is located between 2540 and 2628, intron 12 is located between 2629 and 2683, exon 13 is located between 2684 and 2829, intron 13 is located between 2830 and 2887, exon 14 is located between 2888 and 2958, intron 15 is located between 2959 and 3025, exon 15 is located between 3026 and 3130, intron 15 is located between 3131 and 3208, and exon 16 is located between 3209 and 3325. A region represented by nucleotide No. 3326 and forward is a 3′-nontranslation region including a terminator.

Moreover, it was found that an amino acid sequence estimated from analysis of the nucleotide sequence is an amino acid sequence consisting of 768 amino acid residues, which is shown in SEQ ID No. 4.

Furthermore, in the cellobiose dehydrogenase 1 gene, a cellobiose dehydrogenase gene portion ranging from nucleotide 129 to nucleotide 3274 of a nucleotide sequence as shown in SEQ ID No. 1 is useful. In the cellobiose dehydrogenase 2 gene, a cellobiose dehydrogenase gene portion ranging from nucleotide 159 to nucleotide 3325 of a nucleotide sequence as shown in SEQ ID No. 3 is particularly useful.

A DNA fragment of the cellobiose dehydrogenase gene derived from Coriolus hirsutus can be obtained from the DNA fragment containing the above-described cellobiose dehydrogenase chromosomal gene by PCR. As primers used in PCR, sequences consisting of approximately 10 to 50 nucleotides, and preferably consisting of approximately 15 to 30 nucleotides, which are obtained based on the above-described nucleotide sequences shown in SEQ ID NOS: 1 and 3 and sequences complementary thereto, can be used as a sense primer and an antisense primer. For example, a sense primer shown in SEQ ID No. 31 and an antisense primer shown in SEQ ID No. 32 can be used (refer to Example 12).

It is to be noted that transformed Escherichia coli strains, Escherichia coli JM109/pCHCDH1 and Escherichia coli JM109/pCHCDH2, which have genome DNA containing the sequence of the cellobiose dehydrogenase gene derived from Coriolus hirsutus, were deposited with the National Institute of Advanced Industrial Science and Technology, an Independent Administrative Institution under the Ministry of Economy, Trade and Industry (the AIST Tsukuba Central 6, Higashi 1-1-1, Tsukuba, Ibaraki, Japan) under accession Nos. FERM BP-8278 and FERM B-8279, respectively, on Feb. 8, 2002. A DNA comprising the nucleotide sequence as shown in SEQ ID No. 1 or 3 contained in these deposited strains is also included in the present invention.

The cellobiohydrolase I gene described in 2. above can be obtained by the following procedure.

Plaque hybridization is carried out in the same manner as in preparation of the gene described 1. above, so as to prepare clones containing cellobiohydrolase I genes. A DNA fragment containing the cellobiohydrolase I gene is isolated from the selected clones, followed by preparation of a restriction map thereof, and determination of a sequence thereof. Such sequencing can be carried out by inserting a DNA fragment containing the cellobiohydrolase I gene into a suitable cloning vector (e.g., a pUC vector such as pUC19), and then applying the method of Sanger et al. (as described above).

According to the above-described procedure, nucleotide sequences shown in SEQ ID NOS: 7, 9, and 11 encoding cellobiohydrolase I derived from Coriolus hirsutus have been determined. The thus determined nucleotide sequences were named as cellobiohydrolase I-1 gene, cellobiohydrolase 1-2 gene, and cellobiohydrolase 1-3 gene, respectively.

Moreover, it was found that amino acid sequences estimated from analysis of the above nucleotide sequences were an amino acid sequence as shown in SEQ ID No. 8 consisting of 456 amino acid residues, an amino acid sequence as shown in SEQ ID No. 10 consisting of 456 amino acid residues, and an amino acid sequence as shown in SEQ ID No. 12 consisting of 457 amino acid residues, respectively.

DNA fragments of the above-described cellobiohydrolase I-1 to I-3 genes can be obtained from DNA fragments containing chromosomal genes of the above-described cellobiohydrolase I-1 to I-3 by PCR. As PCR primers, sequences consisting of approximately 10 to 50 nucleotides, and preferably consisting of approximately 15 to 30 nucleotides, which are obtained based on the above-described nucleotide sequences (SEQ ID NOS: 7, 9, and 11) and sequences complementary thereto, can be used as a sense primer and an antisense primer.

The cellobiohydrolase II gene described in 3. above can be obtained by the following procedure.

Plaque hybridization is carried out in the same manner as in preparation of the gene described 1. above, so as to prepare clones containing cellobiohydrolase II genes. A DNA fragment containing the cellobiohydrolase II gene is isolated from the selected clones, followed by preparation of a restriction map thereof, and determination of a sequence thereof. Such sequencing can be carried out by inserting a DNA fragment containing the cellobiohydrolase II gene into a suitable cloning vector (e.g., a pUC vector such as pUC19), and then applying the method of Sanger et al. (as described above).

According to the above-described procedure, a nucleotide sequence as shown in SEQ ID No. 14 encoding cellobiohydrolase II derived from Coriolus hirsutus has been determined. Moreover, it was found that an amino acid sequence estimated from analysis of the above nucleotide sequence was an amino acid sequence as shown in SEQ ID No. 15 consisting of 453 amino acid residues.

A DNA fragment of the above-described cellobiohydrolase II gene can be obtained from a DNA fragment containing a cellobiohydrolase II chromosomal gene by PCR. As PCR primers, sequences consisting of approximately 10 to 50 nucleotides, and preferably consisting of approximately 15 to 30 nucleotides, which are obtained based on the above-described nucleotide sequence and a sequence complementary thereto, can be used as a sense primer and an antisense primer.

The endoglucanase gene belonging to glycolytic enzyme family 61 described in 4. above can be obtained by the following procedure.

mRNA is recovered from cell bodies obtained by growing Coriolus hirsutus on woodchips, and a cDNA library is then produced according to common methods. In order to isolate an endoglucanase gene belonging to glycolytic enzyme family 61 from the obtained cDNA library, plaques are formed on an appropriate agar medium, and several plaques are randomly isolated therefrom. Thereafter, a cDNA portion derived from Coriolus hirsutus is amplified with two types of suitable primers, and a nucleotide sequence thereof is analyzed, so as to isolate a DNA fragment containing an endoglucanase gene belonging to glycolytic enzyme family 61.

According to the above-described procedure, a nucleotide sequence as shown in SEQ ID No. 18 encoding engoglucanase belonging to glycolytic enzyme family 61 has been determined. Moreover, it was found that an amino acid sequence estimated from analysis of the above nucleotide sequence is an amino acid sequence as shown in SEQ ID No. 19 consisting of 374 amino acid residues.

A DNA fragment of an endoglucanase gene belonging to glycolytic enzyme family 61 can be obtained from a DNA fragment containing the above-described endoglucanase gene belonging to glycolytic enzyme family 61 by PCR. As PCR primers, sequences consisting of approximately 10 to 50 nucleotides, and preferably consisting of approximately 15 to 30 nucleotides, which are obtained based on the nucleotide sequence as shown in SEQ ID No. 18 and a sequence complementary thereto, can be used as a sense primer and an antisense primer.

Moreover, the endoglucanase gene belonging to glycolytic enzyme family 12 described in 5. above can be obtained by the following procedure.

Plaques are randomly isolated from a cDNA library in the same manner as in preparation of the gene described in 4. above. Thereafter, a cDNA portion derived from Coriolus hirsutus is amplified, and a nucleotide sequence thereof is analyzed, so as to isolate an endoglucanase gene belonging to glycolytic enzyme family 12.

According to the above-described procedure, a nucleotide sequence as shown in SEQ ID No. 20 encoding engoglucanase belonging to glycolytic enzyme family 12 has been determined. Moreover, it was found that an amino acid sequence estimated from analysis of the above nucleotide sequence is an amino acid sequence as shown in SEQ ID No. 21 consisting of at least 215 amino acid residues.

A DNA fragment of an endoglucanase gene belonging to glycolytic enzyme family 12 derived from Coriolus hirsutus can be obtained from a DNA fragment containing the above-described endoglucanase gene belonging to glycolytic enzyme family 12 by PCR. As PCR primers, sequences consisting of approximately 10 to 50 nucleotides, and preferably consisting of approximately 15 to 30 nucleotides, which are obtained based on the nucleotide sequence as shown in SEQ ID No. 20 and a sequence complementary thereto, can be used as a sense primer and an antisense primer.

Furthermore, the endoglucanase gene belonging to glycolytic enzyme family 5 described in 6. above can be obtained by the following procedure.

A Basidiomycete, Phanerochaete chrysosporium, is cultured in an appropriate medium, and chromosomal DNA is then recovered by the method of Yelton et al. described in 1. above. With respect to the obtained chromosomal DNA, two suitable PCR primers are produced, which are predicted from the database of the choromosomal DNA of Phanerochaete chrysosporium. Thereafter, a DNA fragment of interest is amplified by conventional methods, so as to obtain an endoglucanase gene belonging to glycolytic enzyme family 5.

According to the above-described procedure, a nucleotide sequence as shown in SEQ ID No. 24 encoding engoglucanase belonging to glycolytic enzyme family 5 derived from Phanerochaete chrysosporium has been determined. Moreover, it was found that an amino acid sequence estimated from analysis of the above nucleotide sequence is an amino acid sequence as shown in SEQ ID No. 25 consisting of 386 amino acid residues.

A DNA fragment of an endoglucanase gene belonging to glycolytic enzyme family 5 derived from Phanerochaete chrysosporium can be obtained from a DNA fragment containing the above-described endoglucanase gene belonging to glycolytic enzyme family 5 by PCR. As PCR primers, sequences consisting of approximately 10 to 50 nucleotides, and preferably consisting of approximately 15 to 30 nucleotides, which are obtained based on the nucleotide sequence as shown in SEQ ID No. 24 and a sequence complementary thereto, can be used as a sense primer and an antisense primer.

Still further, the endoglucanase gene belonging to glycolytic enzyme family 9 described in 7. above can be obtained by the following procedure.

Clones containing endoglucanase genes belonging to glycolytic enzyme family 9 are prepared using suitable PCR primers in the same manner as in preparation of the gene described in 6. above. A DNA fragment containing the endoglucanase gene belonging to glycolytic enzyme family 9 is isolated from the selected clones, followed by preparation of a restriction map thereof, and determination of a sequence thereof. Such sequencing can be carried out by inserting a DNA fragment containing the endoglucanase gene belonging to glycolytic enzyme family 9 into a suitable cloning vector (e.g., a pUC vector such as pUC19), and then applying the method of Sanger et al. (as described above).

According to the above-described procedure, a nucleotide sequence as shown in SEQ ID No. 28 encoding engoglucanase belonging to glycolytic enzyme family 9 derived from Phanerochaete chrysosporium has been determined. Moreover, it was found that an amino acid sequence estimated from analysis of the above nucleotide sequence is an amino acid sequence as shown in SEQ ID No. 29 consisting of 592 amino acid residues.

A DNA fragment of an endoglucanase gene belonging to glycolytic enzyme family 9 can be obtained from a DNA fragment containing the above-described endoglucanase gene belonging to glycolytic enzyme family 9 by PCR. As PCR primers, sequences consisting of approximately 10 to 50 nucleotides, and preferably consisting of approximately 15 to 30 nucleotides, which are obtained based on the nucleotide sequence as shown in SEQ ID No. 28 and a sequence complementary thereto, can be used as a sense primer and an antisense primer.

In the first step of the present method, there is prepared DNA encoding antisense RNA substantially complementary to the whole or a part of a transcription product of a cellulolytic enzyme gene derived from the above-described Basidiomycete.

In the present specification, the term “antisense RNA” is used to mean a nucleotide sequence comprising a sequence substantially complementary to the whole or a part of mRNA as a transcription product of the above cellulolytic enzyme gene, wherein, in a case where it exists in a cell, it binds to the mRNA of a cellulolytic enzyme gene that is complementary thereto, and it thereby inhibits the translation of the cellulolytic enzyme gene and suppresses its expression.

Moreover, the term “substantially” is used herein to mean that as long as the antisense RNA binds to mRNA to form a double strand and it inhibits the translation of mRNA into a protein, the sequence may comprise a mutation such as deletion, substitution, or addition.

The length of the above sequence may be appropriately determined, as long as it is capable of suppressing the expression of any one of the cellulolytic enzyme genes of the present invention. It is not necessarily the same as the length of the entire nucleotide sequence of a cellulolytic enzyme gene. For example, when the expression of a cellulolytic enzyme gene is suppressed, the sequence preferably contains a nucleotide sequence, which encodes amino acids at positions 80 and 128 in SEQ ID NOS: 2 and 4 that are heme-binding sites, and amino acids between positions 236 and 241 in SEQ ID NOS: 2 and 4 that correspond to a flavine adenine dinucleotide (FAD)-binding site.

Preparation of antisense RNA and use of the sequence are carried out by conventional methods known to a person skilled in the art. More specifically, the antisense RNA of a cellulolytic enzyme gene can be obtained by performing PCR to obtain an exon portion of the nucleotide sequence of the cellulolytic enzyme gene, or by digesting the cellulolytic enzyme gene with appropriate restriction enzymes. Moreover, the antisense RNA can also be obtained from the cDNA of the cellulolytic enzyme gene. Furthermore, this antisense RNA may be synthetic RNA that is artificially produced on the basis of the information on the nucleotide sequence of the cellulolytic enzyme gene.

In the second step of the present method, there is prepared a vector comprising (a) DNA encoding the antisense RNA as obtained above, or (b) recombinant DNA comprising DNA encoding the antisense RNA as obtained above and a DNA fragment having a promoter activity, wherein the above DNA ligates to the above DNA fragment such that antisense RNA of a cellulolytic enzyme gene is generated as a result of transcription.

In the present specification, the expression “such that antisense RNA of a cellulolytic enzyme gene is generated as a result of transcription” is used to mean that when DNA encoding antisense RNA is transcribed into mRNA under the action of a promoter in a host, antisense RNA capable of binding to mRNA from the cellulolytic enzyme gene of the present invention to form a double strand and thereby suppressing the expression of the cellulolytic enzyme gene can be generated.

In order that DNA binds to a DNA fragment such that antisense RNA is generated, DNA encoding antisense RNA may be ligated to the downstream of a DNA fragment having a promoter sequence in an antisense direction (reverse direction), and it may be then transcribed into mRNA by the action of the promoter. The obtained mRNA is antisense RNA of the nucleotide sequence of a cellulolytic enzyme gene.

A promoter gene is not particularly limited as long as it is a gene fragment having a function as a promoter, but any types of genes can be used as a promoter gene. Examples of a promoter gene may include a GPD promoter and a ras gene promoter. These promoter genes can be obtained by a known genomic cloning method or PCR method, based on sequences registered in gene banks, sequences described in publications, etc. Otherwise, with regard to deposited genes, those obtainable as a result of request for furnishment can also be used.

A gene containing a promoter sequence and a cellulolytic enzyme gene or DNA encoding the antisense RNA of the above gene can be subjected to introduction of a restriction site, a blunt-end treatment, or a sticky-end treatment, if necessary, and then, they can be ligated to each other using suitable DNA ligase. As recombinant DNA techniques including cloning, a ligation reaction, PCR, or the like, those described in, for example, J. Sambrook et al., Molecular Cloning, A Laboratory Manual, Second Edition, Cold Spring Harbor Laboratory Press, 1989, and Short Protocols In Molecular Biology, Third Edition, A Compendium of Methods from Current Protocols in Molecular Biology, John Wiley & Sons, Inc. can be used.

The type of a vector is not particularly limited. It is selected depending on the type of a host transformed with the vector. As a vector, those capable of autonomously replicating in prokaryotic or eukaryotic host cells or capable of homologously recombining in a chromosome can be used. Examples of such a vector may include plasmids, viruses including phages, and cosmids. A vector may appropriately contain a selective marker, a replication origin, a terminator, a polylinker, an enhancer, a ribosome-binding site, etc. Various vectors used for prokaryotes and eukaryotes, such as bacteria, Mycomycetes, yeasts, animals, or plants, are commercially available, or these vectors are described in publications or the like. Using these vectors, DNA or recombinant DNA encoding antisense RNA of the cellobiose dehydrogenase gene of the present invention, can be introduced into a vector.

DNA can be introduced using the technique described in, for example, J. Sambrook et al. (as described above). In order to introduce DNA or recombinant DNA encoding antisense RNA of the cellobiose dehydrogenase gene of the present invention into a vector, as stated above, it should be introduced into a vector such that the antisense RNA of the cellobiose dehydrogenase gene of the present invention is generated as a result of transcription.

In the third step of the present method, transformation is performed with the above vector, so as to prepare host cells having a suppressed cellulolytic enzyme activity.

Any host cells can be used herein, as long as the cells can exhibit a promoter activity in the expression of DNA encoding antisense RNA of the cellobiose dehydrogenase gene of the present invention. Examples of such host cells may include not only fungi such as Basidiomycetes, Eumycetes, or yeasts, but also other eukaryotes (animal cells, plant cells, insect cells, algae, etc.) and prokaryocytes (bacteria, Schizophyceae, etc.). Of these, preferred host cells are Basidiomycetes, and more preferably Coriolus hirsutus. More specifically, for example, an auxotrophic mutant strain OJI-1078 (FERM BP-4210) described later in examples, which lacks ornithine carbamoyltransferase of Coriolus hirsutus, can be used as a host.

Examples of a transformation method may include the calcium chloride/PEG method, the calcium phosphate method, the lithium acetate method, the electroporation, the protoplast method, the spheroplast method, the lipofection, and the Agrobacterium method, but are not limited thereto.

In the fourth step of the present method, the above-described host cells having a suppressed cellulolytic enzyme activity are inoculated on woodchips, and the woodchips are thereby treated.

In the present specification, the term “woodchips” are used to mean chips obtained by mechanically fragmentating lumbers to a size between 2 and 3 cm. Any types of woodchips can be used herein, as long as they can be obtained from trees including conifers such as Pinus, Cryptomeria japonica, Abies, Picea, Douglas fir or Pinus radiata, and broad-leaved trees such as Fagales, Betulaceae, alders, maples, Eucalyptus, Populus, Acacia, lauans or rubber trees, and they can be used as a material for pulp or the like.

Woodchips are treated with host cells having a suppressed cellulolytic enzyme activity. If the host cells having a suppressed cellulolytic enzyme activity can sufficiently grow therein, woodchips can directly be used without subjecting to a pretreatment. However, if the host cells having a suppressed cellulolytic enzyme activity could more easily grow due to a pretreatment whereby other microorganisms are killed, such a pretreatment is preferably carried out to perform disinfection using autoclave or steaming.

A temperature at which woodchips are treated with host cells having a suppressed cellulolytic enzyme activity is preferably between 10° C. and 60° C., and more preferably between 20° C. and 30° C. The water content in woodchips is set at 20% to 80%, and preferably at 30% to 50%. Air does not need to be supplied into woodchips, if the host cells having a suppressed cellulolytic enzyme activity can sufficiently grow without air supply. However, in general, 0.001 to 1 L/(1 min.) of air may be supplied to 1 L of woodchips (hereinafter, air supply unit L/(1 min.) is referred to as vvm), and preferably, 0.01 vvm to 0.1 vvm of air may be supplied to the above volume of woodchips.

The amount of the host cells having a suppressed cellulolytic enzyme activity inoculated into woodchips can be appropriately determined, unless it reduces pulp yield or paper strength.

The host cells having a suppressed cellulolytic enzyme activity may be degraded disintegrated with sterilized water, and they may be then inoculated on woodchips and cultured therein. Otherwise, a medium may be added to woodchips, so as to treat them. Any medium can be used herein, as long as the host cells having a suppressed cellulolytic enzyme activity can grow therein. Examples of such a medium may include carbon sources such as glucose, cellobiose, or amorphous cellulose. In addition, nitrogen sources such as yeast extract, peptone, various types of amino acid, soybean, corn steep liquor, or nitrogen compounds such as various types of inorganic nitrogen may also be used. Moreover, various types of salts, vitamin, mineral, and the like may be appropriately used, if necessary.

In the case of woodchips treated with the host cells having a suppressed cellulolytic enzyme activity, reduction in refining energy or increase in paper strength is observed in the production of mechanical pulp such as thermomechanical pulp (TMP), ground pulp (GP), or refiner ground pulp (RGP), and improvement of cooking or increase in paper strength is achieved in the production of chemical pulp such as kraft pulp or sulfite pulp.

In addition, the host cells having a suppressed cellulolytic enzyme activity used in the method of the present invention are also useful in the production of cellulolytic enzymes comprising the cultivation in a suitable medium and the recovery of the generated cellulolytic enzymes.

In this case, when cellulolytic enzyme is expressed or translated in a fusion form with a signal peptide, it is generated in the form of secretion, and it can be directly isolated from a medium. In contrast, when cellulolytic enzyme is generated in the form of nonsecretion, cells may be separated and then disintegrated by a treatment such as ultrasonification or homogenizing to obtain a cell extract, and cellulolytic enzyme may be isolated from the extract. Isolation and purification of the enzymes can be carried out by applying methods such as solvent extraction, salting out, desalination, organic solvent deposition, ultrafiltration, ion exchange, hydrophobic interaction, HPLC, gel filtration and affinity chromatography, electrophoresis, or chromatofocusing, singly or in combination of several methods.

For example, a GPD promoter, a ras promoter are ligated upstream of the cellulolytic enzyme gene of the present invention, followed by applying a recombinant DNA technique, so as to produce cellulolytic enzymes in large quantity. The above promoters can be prepared, for example, from recombinant Escherichia coli, E. coli JM109/pCHGP (FERM P-15015) containing a GPD promoter gene derived from Coriolus hirsutus, and from recombinant Escherichia coli, E. coli DH5α/pCHRAS (FERM P-17352) containing a ras promoter gene derived from Coriolus hirsutus.

A protein (cellobiose dehydrogenase) encoded by the cellobiose dehydrogenase gene used in the method of the present invention has the following physicochemical properties.

(1) Action

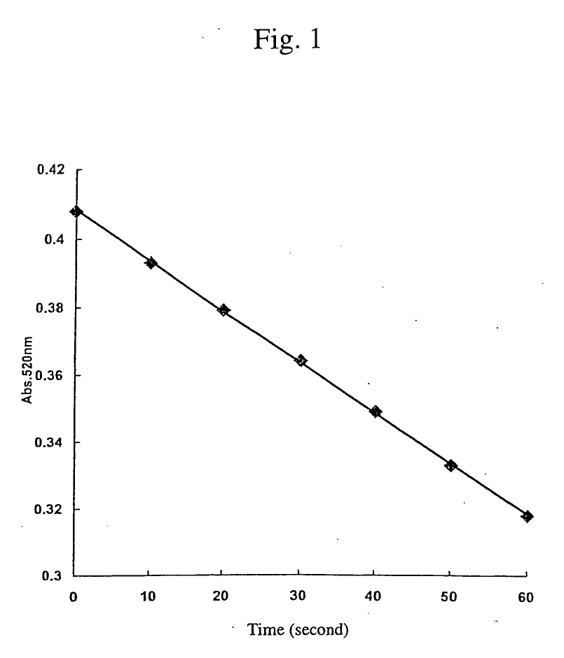

The activity of cellobiose dehydrogenase was measured by the method described in Method in Enzymology (Wood et al, Vol. 160, Academic press, INC. Calif.). That is to say, dichlorophenolindophenol (manufactured by Sigma Chemical Company) and cellobiose (manufactured by Kanto Kagaku) were dissolved in a 50 mM acetate buffer solution of pH 5 such that the concentrations of both components became 0.33 mM and 0.67 mM, respectively. Thereafter, cellobiose dehydrogenase was added to the obtained buffer solution, followed by a reaction at 37° C. After the reaction was started, absorbance (optical length: 1 cm) at 550 nm, the maximum absorption wavelength of dichlorophenolindophenol, was continuously measured. As a result, the results shown in FIG. 1 were obtained.

As shown in FIG. 1, decrease of dichlorophenolindophenol was observed as a result of the reduction reaction of cellobiose dehydrogenase. From the facts that when either cellobiose or cellobiose dehydrogenase was eliminated from the reaction system, decrease of dichlorophenolindophenol was not observed, and that although cytochrome C or Mn(III)-malonic acid complex was used instead of dichlorophenolindophenol, the same reduction reaction was observed, it is clear that the present enzyme is cellobiose dehydrogenase.

(2) Method of Measuring Titer

The activity of cellobiose dehydrogenase was measured as follows. A solution was produced by mixing 250 ul of 0.67 mM dichlorophenolindophenol (manufactured by Sigma Chemical Company), 100 ul of 3.33 mM cellobiose (manufactured by Kanto Kagaku), and 100 ul of a 250 mM acetate buffer solution of pH 5, and thereafter, 50 ul of a test solution was added to the mixed solution, followed by reaction at 37° C. After initiation of the reaction, absorbance (optical length: 1 cm) at 550 nm (molar absorption coefficient: 3965 L/mol/cm) as the maximum absorption wavelength of dichlorophenolindophenol, was continuously measured. With regard to the activity unit of cellobiose dehydrogenase, the amount of enzyme necessary for reducing 1 mmol dichlorophenolindophenol per minute under the above conditions was defined as 1 unit (unit: U).

(3) Substrate Specificity

Cellobiose dehydrogenase acts not only on cellobiose and cellooligosaccharide, but also on Avicel containing cellulose and hardwood tree Kraft pulp.

(4) Optimal pH and Stable pH Range

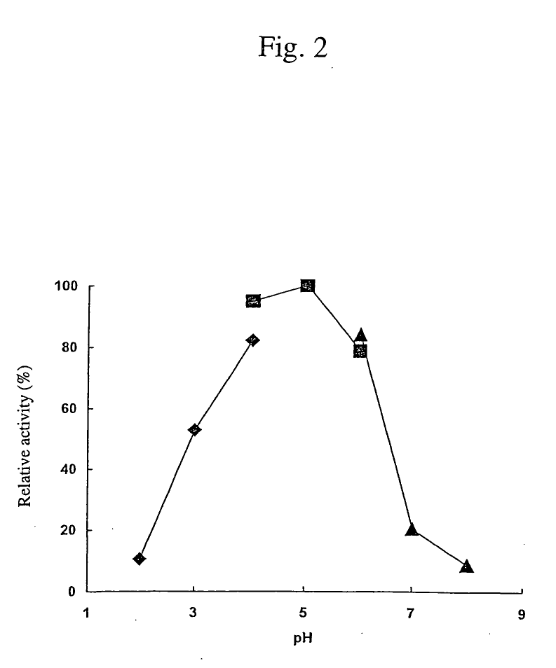

Optimal reaction pH and pH stability were measured using a glycine-HCl buffer solution (pH 2 to 4), an acetate buffer solution (pH 4 to 6), and a phosphate buffer solution (pH 6 to 8). Enzyme activity was measured at the above each pH. The results are shown in FIG. 2.

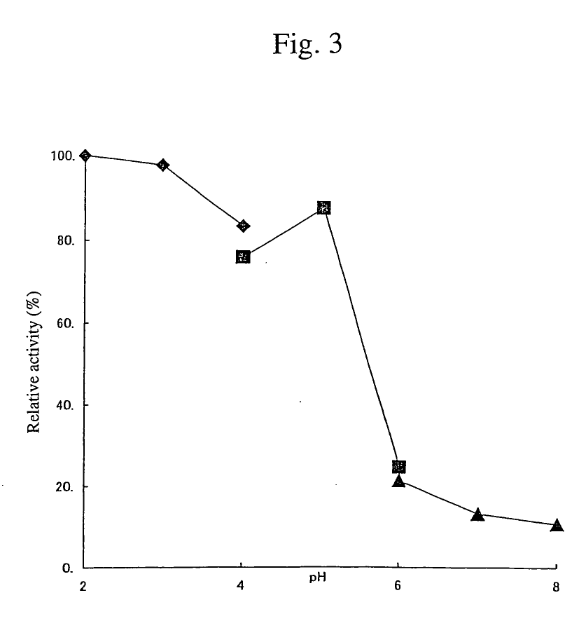

From FIG. 2, it was found that the optimal pH for the enzyme reaction was between pH 4 and pH 6. In addition, the enzyme was incubated at 4° C. for 24 hours in 50 mM each buffer solution, and then enzyme activity was measured. The results are shown in FIG. 3. The present enzyme was stable between pH 2 and pH 5.

(5) Range of Temperatures Suitable for Action

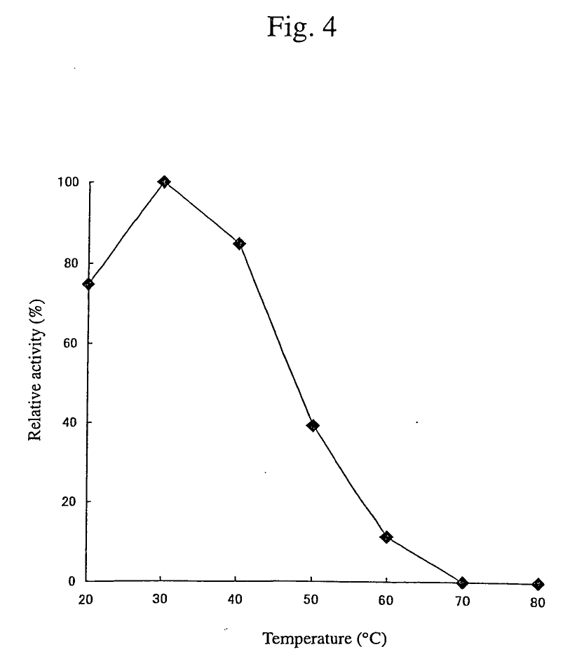

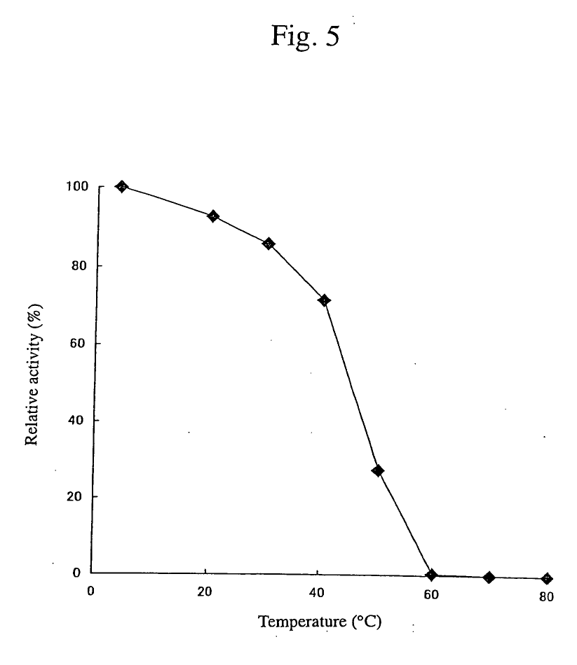

The activity of the enzyme was measured while changing a reaction temperature. The results are shown in FIG. 4. The present enzyme showed a high activity in a temperature range between 20° C. and 40° C. In addition, the enzyme was incubated in a 50 mM acetate buffer solution (pH 5) at a certain temperature for 30 minutes, and then, enzyme activity was measured. The results are shown in FIG. 5.

From FIG. 5, it was found that the enzyme maintained approximately 80% or more activity after a treatment at 40° C. for 30 minutes and that it maintained approximately 30% or more activity even after a treatment at 50° C. for 30 minutes.

(6) Isoelectric Point

Isoelectric focusing was carried out using PRECOAT manufactured by SERVA Electrophoresis GmbH at pH 3 to pH 10. As a result, it was found that the isoelectric point was 4.2.

(7) Molecular Weight

The molecular weight was measured by SDS polyacrylamide gel electrophoresis. As a result, it was found that the molecular weight was approximately 91,700.

(8) Influence by Metal Ions or Inhibitors

Various substances such as metal salts or inhibitors were added to an enzyme solution such that the concentration became 1 mM, followed by incubation at 4° C. overnight. Thereafter, the same types of metal salts were added also into the reaction solution such that the concentration became 1 mM, and enzyme activity was then measured. The results are shown in Table 1. From Table 1, it was found that the enzyme was weakly inhibited by sodium azide and EDTA and was strongly inhibited by Hg²⁺ and SDS.

TABLE 1

| Metal salts | Relative activity |
|---|---|
| (1 mM) | (%) |
| None | 100 |
| FeSO₄ | 0 |
| ZnSO₄ | 72 |
| CuSO₄ | 82 |
| BaCl₂ | 71 |
| MgCl₂ | 64 |
| CaCl₂ | 65 |
| CoCl₂ | 53 |
| MnCl₂ | 35 |
| AlCl₂ | 23 |
| HgCl₂ | 0 |
| NiCl₂ | 60 |
| LiCl₂ | 45 |
| ZnCl₂ | 32 |
| CuCl₂ | 53 |
| NaN₃ | 46 |
| EDTA | 45 |
| SDS | 0 |

In contrast, there have been the following reports regarding the previously known cellobiose dehydrogenase.

Henriksson et al. have reported cellobiose dehydrogenase derived from Phanerochaete chrysosporium, having an optimal reaction pH of 5.0, an isoelectric point of about 4.2, and a molecular weight of 89,000 (Eur. J. Biochem., 196 (1991) 101-106). However, the optimal reaction temperature of this cellobiose dehydrogenase is 50° C.

Roy et al. have reported cellobiose dehydrogenase derived from Trametes versicolor, having an optimal reaction pH of 5.0, an isoelectric point of about 4.2, and a molecular weight of 97,000 (Appl. Environ. Microbiol., 62 (1996) 4417-4427). However, the optimal reaction temperature of this cellobiose dehydrogenase is 50° C.

Fang et al. have reported cellobiose dehydrogenase derived from Schizophyllum commune (Arch. Biochem. Biophys., 353-1 (1998) 37-46). However, the optimal reaction pH of this cellobiose dehydrogenase is 4.5 and the molecular weight thereof is 102,000. They have described neither its optimal temperature nor its isoelectric point.

Schmidhalter, Canevascini, et al., have reported cellobiose dehydrogenase derived from Conephora puteana, having an isoelectric point of about 3.9 (Arch. Biochem. Biophys., 300-2 (1993) 559-563). However, the optimal reaction pH of this cellobiose dehydrogenase is 4.0 and the molecular weight thereof is 111,000. They have not described an optimal reaction temperature.

Canevascini et al. have reported cellobiose dehydrogenase derived from Myceliophtore thermophila, having an isoelectric point of about 4.1 and a molecular weight of 91,000 (Eur. J. Biochem., 198 (1991) 43-52). However, the optimal reaction pH of this cellobiose dehydrogenase is 7.0, and its reaction optimal temperature is not described.

Shou et al. have reported cellobiose dehydrogenase derived from Humicola insolens, having an isoelectric point of about 4.0 and a molecular weight of 92,000 (Biochem. J., 330 (1991) 565-571). The optimal reaction pH of this cellobiose dehydrogenase is 7.0, and the reaction optimal temperature thereof is 65° C.

As stated above, cellobiose dehydrogenase encoded by the cellobiose dehydrogenase gene used in the method of the present invention differs from the known cellobiose dehydrogenases in terms of optimal temperature, optimal pH, molecular weight, and isoelectric point. Accordingly, it is considered that this cellobiose dehydrogenase is a novel cellobiose dehydrogenase. Physiochemical properties of the known cellobiose dehydrogenases are shown in Table 2.

TABLE 2

| | | | | Optimal | |
|---|---|---|---|---|---|
| | Molecular | Isoelectric | Optimal | temperature | |
| | weight (kD) | point | pH | (° C.) | |
| Phanerochaete | 89 | 4.2 | 5.0 | 50 | Eur. J. Biochem., 196(1991) |
| chrysosporium | | | | | 101-106 |
| Trametes versicolor | 97 | 4.2 | 5.0 | 50 | Appl. Environ. Microbiol., |
| | | | | | 62(1996) 4417-4427 |
| Schizophyllum commune | 102 | Not | 4.5 | Not described | Arch. Biochem. Biophys., |
| | | described | | | 353-1(1998) 37-46 |
| Conephora puteana | 111 | 3.9 | 4.0 | Not described | Arch. Biochem. Biophys., |
| | | | | | 300-2(1993) 559-563 |
| Myceliophtore | 91 | 4.1 | 7.0 | Not described | Eur. J. Biochem., 198(1991) |
| thermophila | | | | | 43-52 |
| Humicola insolens | 92 | 4.0 | 7.0 | 65 | Biochem. J., 330(1991) 565-571 |

Coriolus hirsutus having an ability to produce cellobiose dehydrogenase is not particularly limited. For example, a Coriolus hirsutus IFO4917 strain can be used.

Cellobiose dehydrogenase derived from Coriolus hirsutus can be obtained, for example, by culturing Coriolus hirsutus producing cellobiose dehydrogenase in a medium, and collecting cellobiose dehydrogenase from the obtained culture. A medium with any compositions can be used as the above medium for culturing Coriolus hirsutus, as long as the above fingi can proliferate therein. As a nutrient for a medium, those usually used in the culture of Coriolus hirsutus can be widely used. Any carbon sources may be used herein as long as it can be assimilated. Glucose, pulp, crystalline cellulose, etc. can be used as such a carbon source. Any available nitrogen compounds may be used herein as a nitrogen source. For example, yeast extract, peptone, various types of amino acid, soybean, corn steep liquor, various types of inorganic nitrogen can be used as such a nitrogen source. In addition, various types of salts, vitamin, mineral, or the like can be appropriately used, if necessary.

Culture temperature and pH can be appropriately determined within a range where Coriolus hirsutus can proliferate. For example, the culture temperature is between 20° C. and 55° C., and more preferably between 25° C. and 30° C. pH is between 3 and 9, and preferably between 4 and 6.

Cellobiose dehydrogenase derived from Coriolus hirsutus is produced as a secretion product in a culture solution as a result of the culture of Coriolus hirsutus under the above-described conditions. Accordingly, the present enzyme is collected from a culture product obtained as a result of culture. A solution collected from the culture product can be directly used as a cellobiose dehydrogenase crude enzyme solution. However, cellobiose dehydrogenase can also be concentrated or consolidated by salting out, ultrafiltration, or freeze drying. Moreover, cellobiose dehydrogenase can be purified by ammonium sulfate fractionation, molecular weight fractionation by gel filtration, various types of ion exchange resin, hydroxyapatite, hydrophobic chromatogram, isoelectric fractionation, or the like. These methods can be repeated, and further, the methods can be used in combination of other purification methods, if necessary.

### EXAMPLES

The present invention will be more specifically described in the following examples. However, these examples are not intended to limit the scope of the present invention.

### Example 1

### Preparation of Chromosomal DNA Library Derived from Coriolus hirsutus

A Coriolus hirsutus IFO 4917 strain was cultured in an agar plate medium, and an agar section with a diameter of 5 mm was cut out of the culture using a cork borer. It was then inoculated into 200 ml of a glucose-peptone medium (which contained 2% glucose, 0.5% polypeptone, 0.2% yeast extract, KH₂PO₄, and 0.05% MgSO₄, and which was adjusted to pH 4.5 with phosphoric acid), followed by rotary shaking at 28° C. for 7 days. After completion of the culture, cell bodies were collected and then washed with 1 L of sterilized water. Thereafter, the cell bodies were frozen with liquid nitrogen.

5 g of the frozen cell bodies were crushed in a mortar. The crushed cell bodies were transferred into a centrifuge tube, and then, 10 ml of a lytic buffer solution (100 mM Tris (pH 8), 100 mM EDTA, 100 mM NaCl, and proteinase K added such that it became 100 μg/ml) was added thereto, followed by incubation at 55° C. for 3 hours. After completion of the incubation, a phenol treatment and a chloroform treatment were carried out. Ethanol was gradually added to a water phase. When DNA was deposited, chromosomal DNA was taken up and then suspended in a TE solution.

100 μg of the obtained chromosomal DNA was partially decomposed with restriction enzyme Sau3AI, and then fractionated by 5% to 20% sucrose density gradient ultracentrifugation (30,000 rpm, 18 hours), so as to collect a fragmental fraction with a size between 20 and 40 kbp. This fragmental fraction was ligated to phage λ EMBL3-Bam arm manufactured by Toyobo Co., Ltd., using T4 DNA ligase. The obtained phage DNA was packaged with Gigapack Gold manufactured by STRATAGENE. Thereafter, Escherichia coli P2329 was infected with the obtained product, so as to obtain a chromosomal DNA library.

### Example 2

### Isolation of Cellobiose Dehydrogenase Gene from Chromosomal DNA Library

Clones containing cellobiose dehydrogenase genes were selected from the above chromosomal DNA library by plaque hybridization. A series of operations were carried out according to a conventional method (Sambrook et al., Molecular Cloning A Laboratory Manual/2nd Edition (1989)). A probe used in the plaque hybridization was obtained by labeling the 3′-terminus of a synthetic oligomer having the following sequence with fluorescein, using an oligo DNA labeling kit manufactured by Amersham.

| | |
|---|---|
| 5′-TA(T/C)GA(A/G)AA(T/C)AA(A/G)ATT(T/C/A)TT(T/C/A/G)-3′ | (SEQ ID No. 5) |

As a result, 4 positive clones could be selected from approximately 40,000 plaques. Recombinant phage DNA was prepared from the positive clones by conventional methods, and it was then digested with various types of restriction enzymes, followed by Southern hybridization using the above synthetic DNA. As a result, two different clones which hybridize with probes, were observed as DNA bands with sizes of 5.3 kbp and 7.2 kbp in a fragment obtained by digestion with restriction enzyme XhoI.

The above DNA fragments with sizes of 7.2 kbp and 5.3 kbp were cut out by agarose gel electrophoresis, and they were then subcloned into the XhoI site of an Escherichia coli vector pBluescriptII SK+. Thereafter, an Escherichia coli JM109 strain was transformed with the vector. The subcloned DNA was prepared in large quantity, and it was then purified by ultracentrifugation. (50,000 rpm, 16 hours, 15° C.), followed by sequencing. The nucleotide sequences were determined using a sequencing kit manufactured by United States Biochemical.

The nucleotide sequences are shown in SEQ ID NOS: 1 and 3. It was found that the cellobiose dehydrogenase gene derived from Coriolus hirsutus was fragmentated by 15 introns within the range of the above nucleotide sequences. In addition, it was confirmed that amino acid sequences (SEQ ID NOS: 2 and 4) estimated from the nucleotide sequences had high similarity to those of cellobiose dehydrogenase genes that had been reported so far.

### Example 3

### Isolation of Cellobiohydrolase I-1 Gene from Chromosomal DNA Library

Plaque hybridization was carried out in the same manner as in Example 2. A probe used herein was obtained by labeling with fluorescein the 3′-terminus of a synthetic oligomer having the following sequence prepared based on the nucleotide sequence of the cellobiohydrolase I gene isolated from other organisms, using an oligo DNA labeling kit manufactured by Amersham.

(SEQ ID No. 6)

5′-GA(T/C)ATCAAGTT(T/C)ATC(A/G)ATGG-3′

As a result, 2 positive clones could be selected from approximately 40,000 plaques. Recombinant phage DNA was prepared from the positive clones by conventional methods, and it was then digested with various types of restriction enzymes, followed by Southern hybridization using the above synthetic DNA. As a result, a clone as which hybridizes with probe was observed as a single DNA band of 3.9-kbp, in a fragment obtained by digestion with restriction enzymes PstI and NheI.

The above 3.9-kbp DNA fragment was cut out by agarose gel electrophoresis, and it was then subcloned into the PstI-SpeI site of an Escherichia coli vector pBluescriptsII SK−. Thereafter, an Escherichia coli JM109 strain was transformed with the vector, so as to obtain a plasmid pCHCBHI26 containing a cellobiohydrolase I-1 gene derived from Coriolus hirsutus. The nucleotide sequence of the subcloned DNA fragment was determined.

The nucleotide sequence is shown in SEQ ID No. 7. It was found that the cellobiohydrolase I-1 gene derived from Coriolus hirsutus was fragmentated by 2 introns within the range of the above nucleotide sequence. In addition, an amino acid sequence estimated from the nucleotide sequence is shown in SEQ ID No. 8.

### Example 4

### Isolation of Cellobiohydrolase I-2 Gene from Chromosomal DNA Library

Plaque hybridization was carried out in the same manner as in Example 2. A probe used herein was obtained by labeling with fluorescein the 3′-terminus of the synthetic oligomer having the nucleotide sequence as shown in SEQ ID No. 6 used in Example 3, using an oligo DNA labeling kit manufactured by Amersham.

As a result, 3 positive clones could be selected from approximately 40,000 plaques. Recombinant phage DNA was prepared from the positive clones by conventional methods, and it was then digested with various types of restriction enzymes, followed by Southern hybridization using the above synthetic DNA. As a result, a clone which hybridizes with a probe was observed as a single DNA band of 4.2-kbp, in a fragment obtained by digestion with restriction enzyme SalI.

The above 4.2-kbp DNA fragment was cut out by agarose gel electrophoresis, and it was then subcloned into the SalI site of an Escherichia coli vector pUC19. Thereafter, an Escherichia coli JM109 strain was transformed with the vector, so as to obtain a plasmid pCHCBHI27 containing a cellobiohydrolase 1-2 gene derived from Coriolus hirsutus. The nucleotide sequence of the subcloned DNA fragment was determined.

The nucleotide sequence is shown in SEQ ID No. 9. It was found that the cellobiohydrolase 1-2 gene derived from Coriolus hirsutus was fragmentated by 2 introns within the range of the above nucleotide sequence. In addition, an amino acid sequence estimated from the nucleotide sequence is shown in SEQ ID No. 10.

### Example 5

### Isolation of Cellobiohydrolase I-3 Gene from Chromosomal DNA Library

Plaque hybridization was carried out in the same manner as in Example 2. A probe used herein was obtained by labeling with fluorescein the 3′-terminus of the synthetic oligomer having the nucleotide sequence as shown in SEQ ID No. 6 used in Example 3, using an oligo DNA labeling kit manufactured by Amersham.

As a result, 2 positive clones could be selected from approximately 40,000 plaques. Recombinant phage DNA was prepared from the positive clones by conventional methods, and it was then digested with various types of restriction enzymes, followed by Southern hybridization using the above synthetic DNA. As a result, a clone which hybridizes with a probe was observed as a single DNA band of 4.6-kbp, in a fragment obtained by digestion with restriction enzymes EcoRI and BamHI.

The above 4.6-kbp DNA fragment was cut out by agarose gel electrophoresis, and it was then subcloned into the EcoRI-BamHI site of an Escherichia coli vector pUC19. Thereafter, an Escherichia coli JM109 strain was transformed with the vector, so as to obtain a plasmid pCHCBHI31 containing a cellobiohydrolase 1-3 gene derived from Coriolus hirsutus. The nucleotide sequence of the subcloned DNA fragment was determined.

The nucleotide sequence is shown in SEQ ID No. 11. It was found that the cellobiohydrolase 1-3 gene derived from Coriolus hirsutus was fragmentated by 2 introns within the range of the above nucleotide sequence. In addition, an amino acid sequence estimated from the nucleotide sequence is shown in SEQ ID No. 12.

### Example 6

### Isolation of Cellobiohydrolase II Gene from Chromosomal DNA Library

Plaque hybridization was carried out in the same manner as in Example 2. A probe used herein was obtained by labeling with fluorescein the 3′-terminus of a synthetic oligomer having the following sequence prepared based on the nucleotide sequence of a cellobiohydrolase II gene isolated from other organisms, using an oligo DNA labeling kit manufactured by Amersham.

| | |
|---|---|
| 5′-CAGTGGGGIGACTGGTGCAAC-3′ | (SEQ ID No. 13) |

As a result, 8 positive clones could be selected from approximately 100,000 plaques. Recombinant phage DNA was prepared from the positive clones by conventional methods, and it was then digested with various types of restriction enzymes, followed by Southern hybridization using the above synthetic DNA. As a result, a clone which hybridized with a probe was observed as a single DNA band of 5.0-kbp, in a fragment obtained by digestion with restriction enzymes EcoRV and NcoI.

In order to recover the above DNA fragment, a 5.0-kbp DNA fragment obtained by digesting with restriction enzyme NcoI, smoothing with a Klenow fragment, and further digesting with EcoRV, was cut out by agarose gel electrophoresis. Thereafter, it was then subcloned into the SmaI site of an Escherichia coli vector pUC19, so as to obtain a plasmid pCHCBHII containing a cellobiohydrolase II gene derived from Coriolus hirsutus. Thereafter, an Escherichia coli JM109 strain was transformed with this plasmid. The nucleotide sequence of the subcloned DNA fragment was determined.

The nucleotide sequence is shown in SEQ ID No. 14. It was found that the cellobiohydrolase II gene derived from Coriolus hirsutus was fragmentated by 6 introns within the range of the above nucleotide sequence. In addition, an amino acid sequence estimated from the nucleotide sequence is shown in SEQ ID No. 15.

### Example 7

### Preparation of Coriolus hirsutus cDNA Library

6 g dry weight of Eucalyptus globulus chips were placed in a glass schale with a diameter of 9.5 cm, and were then sterilized at 121° C. for 15 minutes. A medium was prepared by adding 20 ml of a peptone medium (which contained 1.0% polypeptone, 0.2% yeast extract, KH₂PO₄, and 0.05% MgSO₄, and which was adjusted to pH 4.5 with phosphoric acid) to the thus treated chips. Thereafter, using a cork borer, agar sections each having a diameter of 5 mm were cut out of the agar plate culture product of a Coriolus hirsutus IFO 4917 strain, and the thus obtained 3 agar sections were inoculated into the above obtained medium, followed by static culture at 30° C. for 10 days. After completion of the culture, cell bodies were collected and then frozen with liquid nitrogen.

Thereafter, total RNA was collected from the frozen cell bodies by the guanidine-hydrochloric acid method. Subsequently, poly(A)⁺ RNA was prepared using an Oligotex-dT <super> mRNA Purification kit manufactured by Takara Shuzo Co., Ltd. Thereafter, using a cDNA Synthesis kit manufactured by STRATAGENE, cDNA was synthesized, and an EcoRI site was attached to the 5′-side thereof and an XhoI site was attached to the 3′-side thereof. It was then inserted into the EcoRI-XhoI site of a λZAPII vector, and using an in vitro packaging kit, a cDNA library was produced.

### Example 8

### Isolation of Endoglucanase Gene Belonging to Glycolytic Enzyme Family 61 from Coriolus hirsutus cDNA Library

A cDNA library solution produced in Example 7 was appropriately diluted such that plaques could be isolated on a schale, and then, an Escherichia coli XL1 Blue MRF′ strain was infected with the solution, followed by culture at 37° C. overnight, so that plaques were formed. A single plaque obtained as above was suspended in an SM buffer. Using universal sequencing primers, an M13(−20) primer (GTAAAACGACGGCCAGT, SEQ ID No. 16) and an M13 reverse primer (GGAAACAGCTATGACCATG, SEQ ID No. 17), a PCR reaction was carried out to amplify a cDNA fragment. The nucleotide sequences of the thus obtained cDNA fragments were randomly analyzed. As a result, a cDNA gene encoding endoglucanase belonging to glycolytic enzyme family 61 was discovered. The nucleotide sequence is shown in SEQ ID No. 18, and an amino acid sequence estimated therefrom is shown in SEQ ID No. 19.

### Example 9

### Isolation of Endoglucanase Gene Belonging to Glycolytic Enzyme Family 12 from Coriolus hirsutus cDNA Library

A cDNA library solution produced in Example 7 was appropriately diluted such that plaques could be isolated on a schale, and then, an Escherichia coli XL1 Blue MRF′ strain was infected with the solution, followed by culture at 37° C. overnight, so that plaques were formed. A single plaque obtained as above was suspended in an SM buffer. Using universal sequencing primers, an M13(−20) primer (GTAAAACGACGGCCAGT, SEQ ID No. 16) and an M13 reverse primer (GGAAACAGCTATGACCATG, SEQ ID No. 17), a PCR reaction was carried out to amplify a cDNA fragment. The nucleotide sequences of the thus obtained cDNA fragments were randomly analyzed. As a result, a cDNA gene encoding endoglucanase belonging to glycolytic enzyme family 12 was discovered. The nucleotide sequence is shown in SEQ ID No. 20, and an amino acid sequence estimated therefrom is shown in SEQ ID No. 21.

### Example 10

### Isolation of Endoglucanase Gene Belonging to Glycolytic Enzyme Family 5 from Basidiomycete Phanerochaete chrysosporium Chromosomal DNA

Using a cork borer, agar sections each having a diameter of 5 mm were cut out of the agar plate culture product of a Phanerochaete chrysosporium ATCC 34541 strain, and the 5 sections were inoculated into 100 ml of a glucose-peptone medium (which contained 2% glucose, 0.5% polypeptone, 0.2% yeast extract, KH₂PO₄, and 0.05% MgSO₄, and which was adjusted to pH 4.5 with phosphoric acid), followed by shaking culture at 30° C. for 5 days. After completion of the culture, cell bodies were collected and then frozen with liquid nitrogen. 5 g of the frozen cell bodies were crushed in a mortar. The crushed cell bodies were transferred into a centrifuge tube, and then, 10 ml of a lytic buffer solution (100 mM Tris (pH 8), 100 mM EDTA, 100 mM NaCl, and proteinase K added such that it became 100 μg/ml) was added thereto, followed by incubation at 55° C. for 3 hours. After completion of the incubation, a phenol treatment and a chloroform treatment were carried out. Ethanol was gradually added to a water phase. When DNA was deposited, chromosomal DNA was taken up and then suspended in a TE solution, so as to produce a Phanerochaete chrysosporium chromosomal DNA solution.

Using two DNA primers described below, a PCR reaction was performed on the above Phanerochaete chrysosporium chromosomal DNA solution, so as to obtain an endoglucanase chromosomal gene belonging to glycolytic enzyme family 5.

| | |
|---|---|
| 5′-ATGAAGTTACTTCTTGCTCTC-3′ | (SEQ ID No. 22) |
| 5′-TCACAGGAAGGGTTCGAGTGC-3′ | (SEQ ID No. 23) |

The obtained nucleotide sequence is shown in SEQ ID No. 24. It was found that the endoglucanase gene belonging to glycolytic enzyme family 5 derived from Phanerochaete chrysosporium was fragmentated by 15 introns within the range of the above nucleotide sequence. In addition, an amino acid sequence estimated from the nucleotide sequence is shown in SEQ ID No. 25.

### Example 11

### Isolation of Endoglucanase Gene Belonging to Glycolytic Enzyme Family 9 from Basidiomycete Phanerochaete chrysosporium Chromosomal DNA

Using two primers described below, a PCR reaction was performed on the Phanerochaete chrysosporium chromosomal DNA solution prepared in Example 10, so as to obtain an endoglucanase gene belonging to glycolytic enzyme family 9.

| | |
|---|---|
| 5′-ATGATACCTCTCCGCTCTGC-3′ | (SEQ ID No. 26) |
| 5′-TATCTTCCTGATGCGATTCC-3′ | (SEQ ID No. 27) |

The obtained nucleotide sequence is shown in SEQ ID No. 28. It was found that the endoglucanase gene belonging to glycolytic enzyme family 9 derived from Phanerochaete chrysosporiunm was fragmentated by 7 introns within the range of the above nucleotide sequence. In addition, an amino acid sequence estimated from the nucleotide sequence is shown in SEQ ID No. 29.

### Example 12

### Construction of Expression Vector for Coriolus hirsutus-derived Cellobiose Dehydrogenase Gene, Using Coriolus hirsutus-derived Glyceraldehyde-3-Phosphate Dehydrogenase Promoter

A structural gene region of a cellobiose dehydrogenase gene was ligated downstream of a promoter of Coriolus hirsutus, whereby the original cellobiose dehydrogenase gene was substituted by a glyceraldehyde-3-phosphate dehydrogenase gene promoter region, so as to obtain a cellobiose dehydrogenase gene expression vector.

More specifically, a glyceraldehyde-3-phosphate dehydrogenase chromosomal gene was digested with EcoRI and BamHI, so as to obtain a 3.8-kbp DNA fragment (fragment 1). The fragment 1 was ligated to the EcoRI-BamHI site of a phage vector M13 mp18, using a T4 DNA ligase. An Escherichia coli JM109 strain was transformed therewith, so as to prepare single-stranded phage DNA.

Subsequently, a DNA primer shown in SEQ ID No. 30 was synthesized, and it was then annealed to the above single-stranded phage DNA. Then, only a promoter region of a GPD gene was synthesized by the primer extension method, and it was then digested with restriction enzyme EcoRI (manufactured by Takara Shuzo Co., Ltd.), so as to prepare a 0.9-kbp DNA fragment (fragment 2).

| | |
|---|---|
| 5′-CATGGTGTGTGGTGGATG-3′ | (SEQ ID No. 30) |

On the other hand, in order to extract only a gene region encoding mature enzyme of cellobiose dehydrogenase, using a plasmid pCHCDH1 as a template, a PCR reaction was carried out with primers shown in SEQ ID NOS: 31 and 32 for elongation, so as to obtain a DNA fragment with a size of approximately 3.5 kbp (fragment 3)

| | |
|---|---|
| 5′-AAGTTCAAGAGTCTCCTGT-3′ | (SEQ ID No. 31) |
| 5′-GGTACAGTACTTATCTGTAT-3′ | (SEQ ID No. 32) |

An Escherichia coli vector pUC18 was digested with restriction enzymes EcoRI and SmaI (Takara Shuzo Co., Ltd.), and the above two types of DNA fragments were mixed and ligated to each other using T4 DNA ligase. Thereafter, an Escherichia coli JM109 strain was transformed therewith. A plasmid, into which the above two types of DNA fragments, fragments 2 and 3, had been simultaneously inserted, was isolated from the ampicillin-resistant transformed strain. The plasmid was named as pGPCDH1.

### Example 13

### Construction of Plasmid Having Antisense Sequence of Cellobiose Dehydrogenase Gene

A 0.9-kb glyceraldehyde-3-phosphate dehydrogenase gene promoter region fragment 2 was obtained by the same method as in Example 12. The obtained fragment 2 was ligated to the EcoRI-SmaI site of pUC18, using T4 DNA ligase, and an Escherichia coli JM109 strain was transformed therewith. A plasmid into which the above fragment 2 had been inserted was isolated from the ampicillin-resistant transformed strain. The plasmid was named as pCHGP1. The plasmid pCHGP1 was digested with restriction enzyme NcoI-XbaI, so as to prepare a vector portion (fragment 4).

Moreover, using two primers described below (SEQ ID NOS: 33 and 34), a PCR reaction was performed on the plasmid pCHCDH1 containing the cellobiose dehydrogenase 1 gene derived from Coriolus hirsutus, so as to amplify an approximately 650-bp DNA fragment containing the 8^{th} exon of the cellobiose dehydrogenase gene derived from Coriolus hirsutus. Thereafter, the amplified product was digested with restriction enzymes XbaI and NcoI to obtain a DNA fragment (fragment 5).

(SEQ ID No. 33)

5′-TCTAGATTTACTGGTACCCCAACAACAATG-3′

(SEQ ID No. 34)

5′-CCATGGGTTGATCGACGGGTTGTCAGACACG-3′

The above fragment 4 was mixed with the above fragment 5, and they were ligated to each other with T4 DNA ligase. Thereafter, an Escherichia coli JM109 strain was transformed therewith. A plasmid into which the above fragment 5 had been inserted was isolated from the ampicillin-resistant transformed strain. The plasmid was named as pGPantiCDH1. The plasmid pGPantiCDH1 was digested with restriction enzymes XbaI and HindIII, so as to prepare a vector portion (fragment 6).

Subsequently, using two primers described below (SEQ ID NOS: 35 and 36), a PCR reaction was performed on a plasmid pBSMPOG1 (FERM P-14933) containing a manganese peroxidase gene derived from Coriolus hirsutus, so as to amplify the C-terminal nontranslation region of manganese peroxidase. The amplified product was digested with restriction enzymes XbaI and HindIII to obtain an approximately 1-kb DNA fragment (fragment 7).

| | |
|---|---|
| 5′-TCTAGAGTCACCTCCGT-3′ | (SEQ ID No. 35) |
| 5′-AAGCTTGGGTACTGTG-3′ | (SEQ ID No. 36) |

The above fragment 6 was mixed with the above fragment 7, and they were ligated to each other with T4 DNA ligase. Thereafter, an Escherichia coli JM109 strain was transformed therewith. A plasmid in which the above DNA fragment 7 had been inserted in a forward direction was isolated from the ampicillin-resistant transformed strain. The plasmid was named as pGPCDHAM.

### Example 14

### Construction of Plasmid Having Antisense Sequence of Cellobiohydrolase I Gene Derived from Coriolus hirsutus

The plasmid pGPCDHAM obtained in Example 13 was digested with restriction enzymes NcoI and XbaI, so as to prepare a DNA fragment for vector portion (fragment 8). Subsequently, using two primers described below, a PCR reaction was performed on the plasmid pCHCBHI26 obtained in Example 3, which contained the cellobiohydrolase I gene derived from Coriolus hirsutus, so as to amplify an approximately 750-bp DNA fragment. Thereafter, the amplified product was digested with restriction enzymes NcoI and XbaI to obtain a DNA fragment (fragment 9).

| | |
|---|---|
| 5′-TCTAGAGCCAACCTCGAGGGGTGG-3′ | (SEQ ID No. 37) |
| 5′-CCATGGGAACGTCGAGCCGATGGG-3′ | (SEQ ID No. 38) |

The above fragment 8 was mixed with the above fragment 9, and they were ligated to each other with T4 DNA ligase. Thereafter, an Escherichia coli JM109 strain was transformed therewith. A plasmid into which the above DNA fragment 9 had been inserted was isolated from the ampicillin-resistant transformed strain. The plasmid was named as pGPCBHI26AM.

### Example 15

### Construction of Plasmid Having Antisense Sequence of Cellobiohydrolase I Gene Derived from Coriolus hirsutus

The plasmid pGPCDHAM obtained in Example 13 was digested with restriction enzymes NcoI and XbaI, so as to prepare a DNA fragment for vector portion (fragment 8). Subsequently, using two primers described below, a PCR reaction was performed on the plasmid pCHCBHI27 obtained in Example 4, which contained the cellobiohydrolase I gene derived from Coriolus hirsutus, so as to amplify an approximately 750-bp DNA fragment. Thereafter, the amplified product was digested with restriction enzymes NcoI and XbaI to obtain a DNA fragment (fragment 10).

| | |
|---|---|
| 5′-TCTAGAGCCAACGTCCTCGGCTGG-3′ | (SEQ ID No. 39) |
| 5′-CCATGGGTAGGTCGAGCCGATGGG-3′ | (SEQ ID No. 40) |

The above fragment 8 was mixed with the above fragment 10, and they were ligated to each other with T4 DNA ligase. Thereafter, an Escherichia coli JM109 strain was transformed therewith. A plasmid into which the above DNA fragment 10 had been inserted was isolated from the ampicillin-resistant transformed strain. The plasmid was named as pGPCBHI27AM.

### Example 16

### Construction of Plasmid Having Antisense Sequence of Cellobiohydrolase I Gene Derived from Coriolus hirsutus

The plasmid pGPCDHAM obtained in Example 13 was digested with restriction enzymes NcoI and XbaI, so as to prepare a DNA fragment for vector portion (fragment 8). Subsequently, using two primers described below, a PCR reaction was performed on the plasmid pCHCBHI31 obtained in Example 5, which contained the cellobiohydrolase I gene derived from Coriolus hirsutus, so as to amplify an approximately 750-bp DNA fragment. Thereafter, the amplified product was digested with restriction enzymes NcoI and XbaI to obtain a DNA fragment (fragment 11).

| | |
|---|---|
| 5′-TCTAGAGCCAACGTCCTCGGCTGG-3′ | (SEQ ID No. 41) |
| 5′-CCATGGAGCGTAGGTCGAGCCAATG-3′ | (SEQ ID No. 42) |

The above fragment 8 was mixed with the above fragment 11, and they were ligated to each other with T4 DNA ligase. Thereafter, an Escherichia coli JM109 strain was transformed therewith. A plasmid into which the above DNA fragment 11 had been inserted was isolated from the ampicillin-resistant transformed strain. The plasmid was named as pGPCBHI31AM.

### Example 17

### Construction of Plasmid Having Antisense Sequence of Cellobiohydrolase II Gene Derived from Coriolus hirsutus

The plasmid pGPCDHAM obtained in Example 13 was digested with restriction enzymes NcoI and XbaI, so as to prepare a DNA fragment for vector portion (fragment 8). Subsequently, using two primers described below, a PCR reaction was performed on the plasmid pCHCBHII obtained in Example 6, which contained the cellobiohydrolase I gene derived from Coriolus hirsutus, so as to amplify an approximately 600-bp DNA fragment. Thereafter, the amplified product was digested with restriction enzymes NcoI and XbaI to obtain a DNA fragment (fragment 12).

| | |
|---|---|
| 5′-TCTAGAATCTACCTGAGCCCTTAC-3′ | (SEQ ID No. 43) |
| 5′-CCATGGCTCACTAGTGGCGAGACC-3′ | (SEQ ID No. 44) |

The above fragment 8 was mixed with the above fragment 12, and they were ligated to each other with T4 DNA ligase. Thereafter, an Escherichia coli JM109 strain was transformed therewith. A plasmid into which the above DNA fragment 12 had been inserted was isolated from the ampicillin-resistant transformed strain. The plasmid was named as pGPCBHIIAM.

### Example 18

### Construction of Plasmid Having Antisense Sequence of Endoglucanase Gene Belonging to Family 61 Derived from Coriolus hirsutus

The plasmid pGPCDHAM obtained in Example 13 was digested with restriction enzymes NcoI and XbaI, so as to prepare a DNA fragment for vector portion (fragment 8). Subsequently, using two primers described below, a PCR reaction was performed on the endoglucanase cDNA gene belonging to glycolytic enzyme family 61 derived from Coriolus hirsutus obtained in Example 12, so as to amplify an approximately 600-bp DNA fragment. Thereafter, the amplified product was digested with restriction enzymes NcoI and XbaI to obtain a DNA fragment (fragment 13).

| | |
|---|---|
| 5′-TCTAGAGCTCACGGTTTCATTCATG-3′ | (SEQ ID No. 45) |
| 5′-CCATGGGGTGTAGAGCCCCGGAATG-3′ | (SEQ ID No. 46) |

The above fragment 8 was mixed with the above fragment 13, and they were ligated to each other with T4 DNA ligase. Thereafter, an Escherichia coli JM109 strain was transformed therewith. A plasmid into which the above DNA fragment 13 had been inserted was isolated from the ampicillin-resistant transformed strain. The plasmid was named as pGPEG61AM.

### Example 19

### Construction of Plasmid Having Antisense Sequence of Endoglucanase Gene Belonging to Family 12 Derived from Coriolus hirsutus

The plasmid pGPCDHAM obtained in Example 13 was digested with restriction enzymes NcoI and XbaI, so as to prepare a DNA fragment for vector portion (fragment 8). Subsequently, using two primers described below, a PCR reaction was performed on the endoglucanase cDNA gene belonging to glycolytic enzyme family 12 derived from Coriolus hirsutus obtained in Example 9, so as to amplify an approximately 700-bp DNA fragment. Thereafter, the amplified product was digested with restriction enzymes NcoI and XbaI to obtain a DNA fragment (fragment 13).

| | |
|---|---|
| 5′-TCTAGAGCGGGCCCGTACTCGCTC-3′ | (SEQ ID No. 47) |
| 5′-CCATGGGTAATGTGATTCCTGTCG-3′ | (SEQ ID No. 48) |

The above fragment 8 was mixed with the above fragment 13, and they were ligated to each other with T4 DNA ligase. Thereafter, an Escherichia coli JM109 strain was transformed therewith. A plasmid into which the above DNA fragment 13 had been inserted was isolated from the ampicillin-resistant transformed strain. The plasmid was named as pGPEG12AM.

### Example 20

### Construction of Plasmid Having Antisense Sequence of Endoglucanase Gene Belonging to Family 5 Derived from Phanerochaete chrysosporium

The plasmid pGPCDHAM obtained in Example 13 was digested with restriction enzymes NcoI and XbaI, so as to prepare a DNA fragment for vector portion (fragment 8). Subsequently, using two primers described below, a PCR reaction was performed on the endoglucanase gene belonging to glycolytic enzyme family 5 derived from Phanerochaete chrysosporium obtained in Example 10, so as to amplify an approximately 600-bp DNA fragment. Thereafter, the amplified product was digested with restriction enzymes NcoI and XbaI to obtain a DNA fragment (fragment 15).

| | |
|---|---|
| 5′-TCTAGAATGAAGTACTTCTTGCTC-3′ | (SEQ ID No. 49) |
| 5′-CCATGGCGTTTGGCGTACCGTCTG-3′ | (SEQ ID No. 50) |

The above fragment 8 was mixed with the above fragment 15, and they were ligated to each other with T4 DNA ligase. Thereafter, an Escherichia coli JM109 strain was transformed therewith. A plasmid into which the above DNA fragment 14 had been inserted was isolated from the ampicillin-resistant transformed strain. The plasmid was named as pGPPCEG5AM.

### Example 21

### Construction of Plasmid Having Antisense Sequence of Endoglucanase Gene Belonging to Family 9 Derived from Phanerochaete chrysosporium

The plasmid pGPCDHAM obtained in Example 13 was digested with restriction enzymes NcoI and XbaI, so as to prepare a DNA fragment for vector portion (fragment 8). Subsequently, using two primers described below, a PCR reaction was performed on the endoglucanase gene belonging to glycolytic enzyme family 9 derived from Phanerochaete chrysosporium obtained in Example 11, so as to amplify an approximately 500-bp DNA fragment. Thereafter, the amplified product was digested with restriction enzymes NcoI and XbaI to obtain a DNA fragment (fragment 16).

| | |
|---|---|
| 5′-TCTAGACCCCGGTACAGACGCCGC-3′ | (SEQ ID No. 51) |
| 5′-CCATGGGATGTTAGGAATGATCTG-3′ | (SEQ ID No. 52) |

The above fragment 8 was mixed with the above fragment 16, and they were ligated to each other with T4 DNA ligase. Thereafter, an Escherichia coli JM109 strain was transformed therewith. A plasmid into which the above DNA fragment 15 had been inserted was isolated from the ampicillin-resistant transformed strain. The plasmid was named as pGPPCEG9AM.

### Example 22

### Method for Transforming Coriolus hirsutus

a. Cultivation of Monokaryon Strain

Approximately 30 glass beads each having a diameter of about 6 mm were placed in a 500 ml-volume Erlenmeyer flask. 100 ml of an SMY medium (1% sucrose, 1% malt extract, and 0.4% yeast extract) was dispensed into the above flask and then sterilized. Thereafter, an agar section having a diameter of 5 mm was cut out of an agar plate medium containing a Coriolus hirsutus OJI-1078 strain, using a cork borer. The section was then inoculated into the above SMY medium, followed by static culture at 28° C. for 7 days (preculture). However, in order to fragmentate hypha, the flask was shaken once or twice a day for mixing. Subsequently, 200 ml of an SMY medium was dispensed into a 1 L-volume Erlenmeyer flask, and a rotator was further added thereto, followed by sterilization. Thereafter, the above precultured hypha was collected by filtration with a nylon mesh (with a pore size of 30 μm), and the total amount of the hypha was inoculated into the medium, followed by culture at 28° C. While culturing, the medium was stirred with a stirrer for 2 hours per day, so that the hypha was fragmentated. This culture was carried out for 4 days.

b. Preparation of Protoplasts

The above liquid culture hypha was collected by filtration with a nylon mesh (with a pore size of 30 μm), and the collected hypha was then washed with an osmoregulation solution (0.5 M MgSO₄, 50 ml maleate buffer (pH 5.6)). Subsequently, 100 mg of wet cell bodies was suspended in 1 ml of a cell-wall-digesting enzyme solution. While the mixture was gently shaken, it was incubated at 28° C. for 3 hours, and protoplasts were released. As a cell-wall-digesting enzyme solution, the following commercially available enzyme preparations were used in combination. This is to say, 5 mg of Cellulase ONOZUKA (cellulase ONOZUKA RS manufactured by Yakult) and 10 mg of Yatalase (manufactured by Takara Shuzo Co., Ltd.) were dissolved in 1 mg of the above osmoregulation solution, and the obtained solution was used as an enzyme solution.

c. Purification of Protoplasts

Hypha fragments were removed from the above enzyme reaction solution using a nylon mesh (with a pore size of 30 μm). Thereafter, in order to enhance the recovery rate of protoplasts, hypha fragments and protoplasts remaining on the nylon mesh were washed once with the above osmoregulation solution. The obtained protoplast suspension was centrifuged (1,000 k×g, 5 minutes) to remove the supernatant. The residue was resuspended in 4 ml of 1 M sucrose (20 mM MOPS buffer solution, pH 6.3). The obtained suspension was centrifuged again, and the resultant product was washed twice with the above 1M sucrose solution. The precipitate was suspended in 500 μl of a 1 M sorbitol solution (20 mM MES, pH 6.4) including 40 mM calcium chloride, and the obtained suspension was used as a protoplast solution. This solution was conserved at 4° C.

The protoplast concentration was determined by direct observation with a speculum, using a hemacytometer. All the centrifugal operations were carried out at 1,000×g for 5 minutes at room temperature, using a swing rotor.

d. Transformation

The plasmid pGPCDH1 (2 μg) obtained in Example 12 was added to 100 μl of a protoplast solution with a concentration of 10⁶ cells/100 μl. Moreover, as a selective marker, 0.2 μg of a plasmid pUCR1 containing an ornithine carbamoyltransferase gene derived from Coriolus hirsutus (JP Patent Publication (Kokai) No. 6-054691 A (1994); FERM BP-4201) was added to the above mixed solution, followed by cooling on ice for 30 minutes. Subsequently, a PEG solution (50% PEG3400, 20 mM MOPS (pH 6.4)) was added thereto in an amount equal to the liquid amount, followed by cooling on ice for 30 minutes. Thereafter, the obtained solution was mixed into a minimal agar medium (1% agar) containing 0.5 M sucrose and leucine, and the mixture was dispersed on a plate. The above plate was cultured at 28° C. for several days, so as to obtain a transformant. Thereafter, DNA was prepared from the transformant, and then, it was confirmed by Southern hybridization that a cellobiose dehydrogenase gene expression plasmid pGPCDH1 of interest was incorporated therein.

### Example 23

### Preparation of Coriolus hirsutus Transformant Highly Secreting and Producing Cellobiose Dehydrogenase

The transformant obtained in Example 22 was inoculated into a 300 ml-volume Erlenmeyer flask containing 100 ml of a glucose-peptone medium (which contained 30 g/l glucose, 10 g/l polypeptone, 1.5 g/l KH₂PO₄, 0.5 g/l MgSO₄, and 2 mg/l thiamine hydrochloride, and which was adjusted to pH 4.5 with phosphoric acid), followed by shaking culture at 28° C. at 100 rpm. Using the above-described cellobiose dehydrogenase activity measurement method, the cellobiose dehydrogenase activity was measured with time. The activity of cellobiose dehydrogenase was 0.02 U/ml in a control, but it was 0.2 U/ml in the transformant.

### Example 24

### Selection of Transformant Having Suppressed Cellobiose Dehydrogenase Activity

Transformation was carried out as described in the transformation method in the above Example 22 using the plasmid pGPCDHAM produced in Example 13 instead of the plasmid pGPCDH1. The obtained transformant was inoculated into a 300 ml-volume Erlenmeyer flask containing 100 ml of hardwood oxygen-bleached kraft pulp (LOKP)-peptone medium (which contained 1% LOKP, 0.5% polypeptone, 0.2% yeast extract, 0.15% KH₂PO₄, and 0.05% MgSO₄, and which was adjusted to pH 4.5 with phosphoric acid), followed by shaking culture at 28° C. at 100 rpm. Using the above-described cellobiose dehydrogenase activity measurement method, the cellobiose dehydrogenase activity was measured with time. As a result, it was found that a transformant having at maximum 70% suppressed cellobiose dehydrogenase activity could be obtained.

### Example 25

### Treatment of Woodchips with Transformant Having Suppressed Cellobiose Dehydrogenase

The transformant with a suppressed cellobiose dehydrogenase activity selected in Example 24 was cultured at 28° C. in a potato dextrose agar medium, and the culture product was conserved at 4° C. 5 sections each having a diameter of 5 mm were cut out form the plate using a cork borer. The 5 sections were then inoculated into a 300 ml-volume Erlenmeyer flask containing 100 ml of a glucose-peptone medium (which contained 2% glucose, 0.5% polypeptone, 0.2% yeast extract, KH₂PO₄, and 0.05% MgSO₄, and which was adjusted to pH 4.5 with phosphoric acid), followed by shaking culture at 28° C. at 100 rpm for 1 week. After completion of the culture, cell bodies were filtrated, and a medium remaining in the cell bodies was washed with sterilized water. The cell bodies were mixed with sterilized water, and they were then crushed with a Waring blender for 15 seconds. Thereafter, the cell bodies were inoculated into 1 kg bone-dry weight of Eucalyptus lumbers, such that the dry weight of the cell bodies became 10 mg. After the inoculation, the mixture was well stirred, such that the cell bodies were distributed uniformly. As a culture, a static culture was carried out at 28° C. for 1 week under aeration. Saturated water vapors were aerated whenever necessary, such that the water contents in the chips became 40% to 65%. For aeration, the amount of air discharged was set at 0.01 vvm per chip.

### Example 26

### Production of Mechanical Pulp Using Transformant Having Suppressed Cellobiose Dehydrogenase Activity

Woodchips were prepared from radiata pine lumbers, and the woodchips were treated in the same manner as in Example 25. The treated woodchips were beaten using a laboratory refiner (manufactured by Kumagai Riki KOGYO), and Canadian Standard Freeness was set at 200 ml. Thereafter, handsheets used for physical tests of pulp were prepared in accordance with Tappi test method T205om-81, and the physical tests of handmade pulp sheets were carried out in accordance with Tappi test T220om-83. Electric energy used herein was measured using a wattmeter (Hiokidenki model 3133) and an integration counter (model 3141). The yield of chips was obtained by placing 1 kg bone-dry weight of woodchips containing water into a container, measuring the bone-dry weight of the woodchips before and after the treatment, and then calculating the chip yield by the following formula:
(bone-dry weight after treatment)/(bone-dry weight before treatment)×100

As shown in Table 3 below, use of a transformant having suppressed cellobiose dehydrogenase could control reduction in chip yield and could reduce refining energy. In addition, both tearing strength and bursting strength were increased. In contrast, when woodchips were treated with a wild type strain, effects of reducing refining energy could be obtained, but paper strength was decreased.

TABLE 3
Effects of woodchip treatment with microorganisms having
suppressed cellobiose dehydrogenase activity on mechanical pulp

| | | | Wild-type |
|---|---|---|---|
| | Control | Transformant | strain |
| Chip yield (%) | 99.8 | 98.9 | 94.7 |
| Refining energy (Kw · h/ton) | 2560 | 1792 | 1840 |
| Tear index (mN · m²/g) | 7.92 | 8.21 | 6.95 |
| Burst index (kPa · m²/g) | 1.35 | 1.52 | 1.21 |

### Example 27

### Cooking of Woodchips Treated with Transformant Having Suppressed Cellobiose Dehydrogenase Activity

Woodchips made from Eucalyptus lumbers were treated in the same manner as in Example 25. Thereafter, 400 g bone-dry weight was weighed from the woodchips. A cooking white liquor was added to the woodchips in an autoclave, such that a liquid ratio of 5, a sulfur degree of 30%, and an effective alkali of 17% (as Na₂O) could be achieved. Thereafter, a cooking temperature was set at 150° C., and Kraft cooking was carried out. After completion of the Kraft cooking, a black liquor was separated, and the obtained chips were refined using a high concentration refining machine. Thereafter, the refined chips were subjected to centrifugal dehydration with a filter cloth followed by washing with water, three times. Thereafter, uncooked products were eliminated by screening, and the residue was subjected to centrifugal dehydration, so as to obtain cooked unbleached pulp.

2.0% by mass of NaOH was added to the pulp obtained by the above Kraft cooking, and oxygen gas was then injected therein, followed by a treatment at 100° C. under an oxygen gauge pressure of 0.49 MPa (5 kg/cm²) for 60 minutes.

The above obtained pulp was subjected to a 4-steps bleaching treatment consisting of D-E-P-D sequence, as described below. In the first chlorine dioxide treatment (D), pulp was prepared such that the concentration of the pulp became 10% by mass, and 0.4% by mass of chlorine dioxide was added thereto, followed by a treatment at 70° C. for 40 minutes. Subsequently, the pulp was washed with ion exchanged water and then dehydrated. The concentration of the pulp was adjusted to 10% by mass, and 1% by mass of sodium hydroxide was added to the pulp, followed by an alkali extraction treatment (E) at 70° C. for 90 minutes. Subsequently, the pulp was washed with ion exchanged water and then dehydrated. The concentration of the pulp was adjusted to 10% by mass, and then, 0.5% by mass of hydrogen peroxide and 0.5% by mass of sodium hydroxide were successively added to the pulp, followed by a hydrogen peroxide treatment (P) at 70° C. for 120 minutes. Subsequently, the pulp was washed with ion exchanged water and then dehydrated. The concentration of the pulp was adjusted to 10%, and 0.25% by mass of chlorine dioxide was added to the pulp, followed by a chlorine dioxide treatment (D) at 70° C. for 180 minutes. Finally, the pulp was washed with ion exchanged water and then dehydrated, so as to obtain bleached pulp with a whiteness degree of 86.0% in accordance with JIS P 8123.

The thus obtained pulp slurry having a pulp concentration of 4% by mass was beaten with a refiner, such that the freeness became 410 ml (CSF).

### Example 28

### Measurement of Ka Value, Preparation of Handsheets Used for Physical Tests of Pulp, and Physical Tests of Handmade Pulp Sheets

Kappa value was measured in accordance with JIS P 8211. Handmade sheets used for physical tests of pulp were prepared in accordance with Tappi test method T205-om81, and the physical tests of handmade pulp sheets were carried out in accordance with Tappi test T2200m-83. As shown in Table 4 below, when woodchips were treated with a transformant or wild type strain, the Ka value was decreased after cooking, the screened yield was increased, and the screened rejects was decreased. In addition, as shown in Table 5 below, when the transformant was compared with the wild type strain, the transformant did not cause decrease in paper strength.

TABLE 4
Effects of woodchip treatment with microorganisms having
suppressed cellobiose dehydrogenase activity on cooking

| | Control | Transformant | Wild type strain |
|---|---|---|---|
| Ka value after cooking | 20.1 | 17.6 | 17.7 |
| Screened yield (%) | 45.7 | 47.8 | 47.3 |
| Screened rejects (%) | 1.20 | 0.65 | 0.84 |

TABLE 5
Effects of woodchip treatment with microorganisms having
suppressed cellobiose dehydrogenase activity on paper strength
Whiteness degree = 86, and CSF (Canadian Shopper
Freeness) = 410 ml

| | Control | Transformant | Wild type strain |
|---|---|---|---|
| PFI (rev) | 2,600 | 2,200 | 2,200 |
| Tear index (mN · m²/g) | 9.4 | 9.3 | 8.5 |
| Breaking length (km) | 8.62 | 8.71 | 7.34 |
| Burst index (kPa · m²/g) | 6.76 | 7.72 | 7.54 |
| Folding endurance (logT) | 2.31 | 2.35 | 2.19 |

Note)
Control means a pulp sheet produced by a woodchip treatment wherein no microorganism treatment was carried out.

### Example 29

### Selection of Transformant Having Suppressed Cellobiohydrolase I Activity

Transformation was carried out as described in the transformation method in the above Example 22 using the plasmid pGPCBHI26AM produced in Example 14 instead of the plasmid pGPCDH1. The obtained transformant was cultured by the same method as in Example 24. Thereafter, applying a cellobiohydrolase I activity measurement method using 4-methyl-O-umbellifferyl-cellobioside as a substrate, the activity of cellobiohydrolase I was measured with time. As a result, it was found that a transformant having at maximum 60% suppressed cellobiohydrolase I activity could be obtained.

### Example 30

### Treatment of Woodchips with Transformant Having Suppressed Cellobiohydrolase I Activity

Woodchips made from Eucalyptus lumbers were treated with the transformant strain having a suppressed cellobiohydrolase I activity selected in Example 29 according to the same method as in Example 25.

### Example 31

### Production of Mechanical Pulp Using Transformant Having Suppressed Cellobiohydrolase I Activity

Woodchips made from radiata pine lumbers were treated in the same manner as in Example 25. The treated woodchips were subjected to the same test as in Example 26. As shown in Table 6 below, use of a transformant with a suppressed cellobiohydrolase I activity could control reduction in chip yield and could reduce refining energy. In addition, both tearing strength and bursting strength were increased. In contrast, when woodchips were treated with a wild type strain, effects of reducing refining energy could be obtained, but paper strength was decreased.

TABLE 6
Effects of woodchip treatment with microorganisms having
suppressed cellobiohydrolase I activity on mechanical pulp

| | | | Wild |
|---|---|---|---|
| | Control | Transformant | type strain |
| Chip yield (%) | 99.8 | 98.7 | 94.7 |
| Refining energy (Kw · h/ton) | 2560 | 1782 | 1840 |
| Tear index (mN · m²/g) | 7.92 | 8.02 | 6.95 |
| Burst index (kPa · m²/g) | 1.35 | 1.50 | 1.21 |

### Example 32

### Cooking of Woodchips Treated with Transformant Having Suppressed Cellobiohydrolase I Activity

The woodchips treated in Example 30 were cooked by the same method as in Example 27.

### Example 33

### Measurement of Ka Value, Preparation of Handsheets Used for Physical Tests of Pulp, and Physical Tests of Handmade Pulp Sheets

Measurement of the kappa value or the like was carried out on the pulp obtained in Example 32 in the same manner as described in Example 28. As shown in Table 7 below, when woodchips were treated with a transformant or wild type strain, the Ka value was decreased after cooking, the Screened yield was increased, and the Screened rejects was decreased. In addition, as shown in Table 8 below, when the transformant was compared with the wild type strain, the transformant did not cause decrease in paper strength.

TABLE 7
Effects of woodchip treatment with microorganisms having
suppressed cellobiohydrolase I activity on cooking

| | Control | Transformant | Wild type strain |
|---|---|---|---|
| Ka value after cooking | 20.1 | 17.7 | 17.7 |
| Screened yield (%) | 45.7 | 47.9 | 47.3 |
| Screened rejects (%) | 1.20 | 0.75 | 0.84 |

TABLE 8
Effects of woodchip treatment with microorganisms having
suppressed cellobiohydrolase I activity on paper strength
Whiteness degree = 86, and CSF (Canadian Shopper
Freeness) = 410 ml

| | Control | Transformant | Wild type strain |
|---|---|---|---|
| PFI (rev) | 2,600 | 2,300 | 2,200 |
| Tear index (mN·m²/g) | 9.4 | 9.4 | 8.5 |
| Breaking length (km) | 8.62 | 8.51 | 7.34 |
| Burst index (kPa·m²/g) | 6.76 | 7.82 | 7.54 |
| Folding endurance (logT) | 2.31 | 2.34 | 2.19 |

Note)
Control means a pulp sheet produced by a woodchip treatment wherein no microorganism treatment was carried out.

### Example 34

### Selection of Transformant Having Suppressed Cellobiohydrolase I Activity

Transformation was carried out as described in the transformation method in the above Example 22 using the plasmid pGPCBHI27AM produced in Example 15 instead of the plasmid pGPCDH1. The obtained transformant was cultured by the same method as in Example 26. Thereafter, applying a cellobiohydrolase I activity measurement method using 4-methyl-O-umbellifferyl-cellobioside as a substrate, the activity of cellobiohydrolase I was measured with time. As a result, it was found that a transformant having at maximum 70% suppressed cellobiohydrolase I activity could be obtained.

### Example 35

### Treatment of Woodchips with Transformant Having Suppressed Cellobiohydrolase I Activity

Woodchips made from Eucalyptus lumbers were treated with the transformant strain having a suppressed cellobiohydrolase I activity selected in Example 34 according to the same method as in Example 25.

### Example 36

### Production of Mechanical Pulp Using Transformant Having Suppressed Cellobiohydrolase I Activity

Woodchips made from radiata pine lumbers were treated with the transformant obtained in Example 34 in the same manner as in Example 25. The treated woodchips were subjected to the same test as in Example 26. As shown in Table 9 below, use of a transformant with a suppressed cellobiohydrolase I activity could control reduction in chip yield and could reduce refining energy. In addition, both tearing strength and bursting strength were increased. In contrast, when woodchips were treated with a wild type strain, effects of reducing refining energy could be obtained, but paper strength was decreased.

TABLE 9
Effects of woodchip treatment with microorganisms having
suppressed cellobiohydrolase I activity on mechanical pulp

| | | | Wild |
|---|---|---|---|
| | Control | Transformant | type strain |
| Chip yield (%) | 99.8 | 98.5 | 94.7 |
| Refining energy (Kw · h/ton) | 2560 | 1752 | 1840 |
| Tear index (mN · m²/g) | 7.92 | 8.11 | 6.95 |
| Burst index (kPa · m²/g) | 1.35 | 1.42 | 1.21 |

### Example 37

### Cooking of Woodchips Treated with Transformant Having Suppressed Cellobiohydrolase I Activity

Woodchips made from Eucalyptus lumbers were treated in the same manner as in Example 25. Thereafter, the treated woodchips were cooked by the same method as in Example 27.

### Example 38

### Measurement of Ka Value, Preparation of Handsheets Used for Physical Tests of Pulp, and Physical Tests of Handmade Pulp Sheets

Measurement of the kappa value or the like was carried out on the pulp obtained in Example 37 in the same manner as described in Example 28. As shown in Table 10 below, when woodchips were treated with a transformant or wild type strain, the Ka value was decreased after cooking, the Screened yield was increased, and the Screened rejects was decreased. In addition, as shown in Table 11 below, when the transformant was compared with the wild type strain, the transformant did not cause decrease in paper strength.

TABLE 10
Effects of woodchip treatment with microorganisms having
suppressed cellobiohydrolase I activity on cooking

| | Control | Transformant | Wild type strain |
|---|---|---|---|
| Ka value after cooking | 20.1 | 17.5 | 17.7 |
| Screened yield (%) | 45.7 | 48.2 | 47.3 |
| Screened rejects (%) | 1.20 | 0.45 | 0.84 |

TABLE 11
Effects of woodchip treatment with microorganisms having
suppressed cellobiohydrolase I activity on paper strength
Whiteness degree = 86, and CSF (Canadian Shopper
Freeness) = 410 ml

| | Control | Transformant | Wild type strain |
|---|---|---|---|
| PFI (rev) | 2,600 | 2,300 | 2,200 |
| Tear index (mN · m²/g) | 9.4 | 9.1 | 8.5 |
| Breaking length (km) | 8.62 | 8.54 | 7.34 |
| Burst index (kPa · m²/g) | 6.76 | 7.75 | 7.54 |
| Folding endurance (logT) | 2.31 | 2.31 | 2.19 |

Note)
Control means a pulp sheet produced by a woodchip treatment wherein no microorganism treatment was carried out.

### Example 39

### Selection of Transformant Having Suppressed Cellobiohydrolase I Activity

Transformation was carried out as described in the transformation method in the above Example 22 using the plasmid pGPCBHI31AM produced in Example 16 instead of the plasmid pGPCDH1. The obtained transformant was cultured by the same method as in Example 24. Thereafter, applying a cellobiohydrolase I activity measurement method using 4-methyl-O-umbellifferyl-cellobioside as a substrate, the activity of cellobiohydrolase I was measured with time. As a result, it was found that a transformant having at maximum 70% suppressed cellobiohydrolase I activity could be obtained.

### Example 40

### Treatment of Woodchips with Transformant Having Suppressed Cellobiohydrolase I Activity

Woodchips made from Eucalyptus lumbers were treated with the transformant strain having a suppressed cellobiohydrolase I activity selected in Example 39 according to the same method as in Example 25.

### Example 41

### Production of Mechanical Pulp Using Transformant Having Suppressed Cellobiohydrolase I Activity

Woodchips made from radiata pine lumbers were treated with the transformant obtained in Example 39 in the same manner as in Example 25. The treated woodchips were subjected to the same test as in Example 26.

As shown in Table 12 below, use of a transformant with a suppressed cellobiohydrolase I activity could control reduction in chip yield and could reduce refining energy. In addition, both tearing strength and bursting strength were increased. In contrast, when woodchips were treated with a wild type strain, effects of reducing refining energy could be obtained, but paper strength was decreased.

TABLE 12
Effects of woodchip treatment with microorganisms having
suppressed cellobiohydrolase I activity on mechanical pulp

| | | | Wild type |
|---|---|---|---|
| | Control | Transformant | strain |
| Chip yield (%) | 99.8 | 98.3 | 94.7 |
| Refining energy (Kw · h/ton) | 2560 | 1820 | 1840 |
| Tear index (mN · m²/g) | 7.92 | 8.35 | 6.95 |
| Burst index (kPa · m²/g) | 1.35 | 1.43 | 1.21 |

### Example 42

### Cooking of Woodchips Treated with Transformant Having Suppressed Cellobiohydrolase I Activity

The woodchips obtained in Example 39 were cooked by the same method as in Example 27.

### Example 43

### Measurement of Ka Value, Preparation of Handsheets Used for Physical Tests of Pulp, and Physical Tests of Handmade Pulp Sheets

Measurement of the kappa value or the like was carried out on the pulp obtained in Example 42 in the same manner as described in Example 28. As shown in Table 13 below, when woodchips were treated with a transformant or wild type strain, the Ka value was decreased after cooking, the Screened yield was increased, and the Screened rejects was decreased. In addition, as shown in Table 14 below, when the transformant was compared with the wild type strain, the transformant did not cause decrease in paper strength.

TABLE 13
Effects of woodchip treatment with microorganisms having
suppressed cellobiohydrolase I activity on cooking

| | Control | Transformant | Wild type strain |
|---|---|---|---|
| Ka value after cooking | 20.1 | 17.8 | 17.7 |
| Screened yield (%) | 45.7 | 47.4 | 47.3 |
| Screened rejects (%) | 1.20 | 0.63 | 0.84 |

TABLE 14
Effects of woodchip treatment with microorganisms having
suppressed cellobiohydrolase I activity on paper strength
Whiteness degree = 86, and CSF (Canadian
Shopper Freeness) = 410 ml

| | Control | Transformant | Wild type strain |
|---|---|---|---|
| PFI (rev) | 2,600 | 2,200 | 2,200 |
| Tear index (mN · m²g) | 9.4 | 9.1 | 8.5 |
| Breaking length (km) | 8.62 | 8.61 | 7.34 |
| Burst index (kPa · m²/g) | 6.76 | 7.73 | 7.54 |
| Folding endurance (logT) | 2.31 | 2.35 | 2.19 |

Note)
Control means a pulp sheet produced by a woodchip treatment wherein no microorganism treatment was carried out.

### Example 44

### Selection of Transformant Having Suppressed Cellobiohydrolase II Activity

Transformation was carried out as described in the transformation method in the above Example 22 using the plasmid pGPCBHIIAM produced in Example 17 instead of the plasmid pGPCDH1. The obtained transformant was cultured by the same method as in Example 23. The cultured cell bodies were sampled periodically, and mRNA was recovered, and the amount of the expression of cellobiohydrolase II gene was measured. As a result, it was found that a transformant could be obtained, which had a suppressed cellobiohydrolase II activity that was approximately 50% less than that of the host cells.

### Example 45

### Treatment of Woodchips with Transformant Having Suppressed Cellobiohydrolase II Activity

Woodchips made from Eucalyptus lumbers were treated with the transformant strain having a suppressed cellobiohydrolase II activity selected in Example 44 according to the same method as in Example 25.

### Example 46

### Production of Mechanical Pulp Using Transformant Having Suppressed Cellobiohydrolase II Activity

Woodchips made from radiata pine lumbers were treated with the transformant obtained in Example 44 in the same manner as in Example 25. Analysis of the paper strength or the like was carried out on the treated woodchips according to the method described in Example 26. As shown in Table 15 below, use of a transformant with a suppressed cellobiohydrolase II activity could control reduction in chip yield and could reduce refining energy. In addition, both tearing strength and bursting strength were increased. In contrast, when woodchips were treated with a wild type strain, effects of reducing refining energy could be obtained, but paper strength was decreased.

TABLE 15
Effects of woodchip treatment with microorganisms having
suppressed cellobiohydrolase II activity on mechanical pulp

| | | | Wild type |
|---|---|---|---|
| | Control | Transformant | strain |
| Chip yield (%) | 99.8 | 98.1 | 94.7 |
| Refining energy (Kw · h/ton) | 2560 | 1830 | 1840 |
| Tear index (mN · m²/g) | 7.92 | 8.25 | 6.95 |
| Burst index (kPa · m²/g) | 1.35 | 1.44 | 1.21 |

### Example 47

### Cooking of Woodchips Treated with Transformant Having Suppressed Cellobiohydrolase II Activity

The woodchips obtained in Example 45 were cooked by the same method as in Example 27.

### Example 48

### Measurement of Ka Value, Preparation of Handsheets Used for Physical Tests of Pulp, and Physical Tests of Handmade Pulp Sheets

Measurement of the kappa value or the like was carried out on the pulp obtained in Example 47 in the same manner as described in Example 28. As shown in Table 16 below, when woodchips were treated with a transformant or wild type strain, the Ka value was decreased after cooking, the Screened yield was increased, and the Screened rejects was decreased. In addition, as shown in Table 17 below, when the transformant was compared with the wild type strain, the transformant did not cause decrease in paper strength.

TABLE 16
Effects of woodchip treatment with microorganisms having
suppressed cellobiohydrolase II activity on cooking

| | Control | Transformant | Wild type strain |
|---|---|---|---|
| Ka value after cooking | 20.1 | 18.1 | 17.7 |
| Screened yield (%) | 45.7 | 47.1 | 47.3 |
| Screened rejects (%) | 1.20 | 0.86 | 0.84 |

TABLE 17
Effects of woodchip treatment with microorganisms having
suppressed cellobiohydrolase II activity on paper strength
Whiteness degree = 86, and CSF (Canadian
Shopper Freeness) = 410 ml

| | Control | Transformant | Wild type strain |
|---|---|---|---|
| PFI (rev) | 2,600 | 2,300 | 2,200 |
| Tear index (mN · m²/g) | 9.4 | 8.7 | 8.5 |
| Breaking length (km) | 8.62 | 8.45 | 7.34 |
| Burst index (kPa · m²/g) | 6.76 | 7.49 | 7.54 |
| Folding endurance (logT) | 2.31 | 2.32 | 2.19 |

Note)
Control means a pulp sheet produced by a woodchip treatment wherein no microorganism treatment was carried out.

### Example 49

### Selection of Transformant Having Suppressed Activity of Engoglucanase Belonging to Glycolytic Enzyme Family 61

Transformation was carried out as described in the transformation method in the above Example 22 using the plasmid pGPEG61AM produced in Example 18 instead of the plasmid pGPCDH1. The obtained transformant was cultured by the same method as in Example 23. The culture solution was sampled with time, and the carboxymethyl cellulose (CMC)-decomposing activity was measured. As a result, it was found that a transformant could be obtained, which had a suppressed endoglucanase activity that was approximately 50% less than that of the host cells.

### Example 50

### Treatment of Woodchips with Transformant Having Suppressed Activity of Endoglucanase Belonging to Glycolytic Enzyme Family 61

Woodchips made from Eucalyptus lumbers were treated with the transformant strain having a suppressed activity of endoglucanase belonging to glycolytic enzyme family 61 selected in Example 49 according to the method described in Example 25.

### Example 51

### Production of Mechanical Pulp Using Transformant Having Suppressed Activity of Endoglucanase Belonging to Glycolytic Enzyme Family 61

Woodchips made from radiata pine lumbers were treated with the transformant obtained in Example 49 in the same manner as in Example 25. Analysis of the paper strength or the like was carried out on the treated woodchips by the method described in Example 26. As shown in Table 18 below, use of a transformant with a suppressed activity of endoglucanase belonging to glycolytic enzyme family 61 could control reduction in chip yield and could reduce refining energy. In addition, both tearing strength and bursting strength were increased. In contrast, when woodchips were treated with a wild type strain, effects of reducing refining energy could be obtained, but paper strength was decreased.

TABLE 18
Effects of woodchip treatment with microorganisms
having suppressed activity of endoglucanase belonging
to glycolytic enzyme family 61 on mechanical pulp

| | | | Wild type |
|---|---|---|---|
| | Control | Transformant | strain |
| Chip yield (%) | 99.8 | 98.6 | 94.7 |
| Refining energy (Kw · h/ton) | 2560 | 1800 | 1840 |
| Tear index (mN · m²/g) | 7.92 | 8.21 | 6.95 |
| Burst index (kPa · m²/g) | 1.35 | 1.41 | 1.21 |

### Example 52

### Cooking of Woodchips Treated with Transformant Having Suppressed Activity of Endoglucanase Belonging to Glycolytic Enzyme Family 61

The woodchips obtained in Example 50 were cooked by the same method as in Example 27.

### Example 53

### Measurement of Ka Value, Preparation of Handsheets Used for Physical Tests of Pulp, and Physical Tests of Handmade Pulp Sheets

Measurement of the kappa value or the like was carried out on the pulp obtained in Example 52 in the same manner as described in Example 28. As shown in Table 19 below, when woodchips were treated with a transformant or wild type strain, the Ka value was decreased after cooking, the Screened yield was increased, and the Screened rejects was decreased. In addition, as shown in Table 20 below, when the transformant was compared with the wild type strain, the transformant did not cause decrease in paper strength.

TABLE 19
Effects of woodchip treatment with microorganisms
having suppressed activity of endoglucanase belonging
to glycolytic enzyme family 61 on cooking

| | Control | Transformant | Wild type strain |
|---|---|---|---|
| Ka value after cooking | 20.1 | 17.7 | 17.7 |
| Screened yield (%) | 45.7 | 47.6 | 47.3 |
| Screened rejects (%) | 1.20 | 0.73 | 0.84 |

TABLE 20
Effects of woodchip treatment with microorganisms
having suppressed activity of endoglucanase belonging
to glycolytic enzyme family 61 on paper strength
Whiteness degree = 86, and CSF
(Canadian Shopper Freeness) = 410 ml

| | Control | Transformant | Wild type strain |
|---|---|---|---|
| PFI (rev) | 2,600 | 2,200 | 2,200 |
| Tear index (mN · m²/g) | 9.4 | 9.5 | 8.5 |
| Breaking length (km) | 8.62 | 8.63 | 7.34 |
| Burst index (kPa · m²/g) | 6.76 | 7.63 | 7.54 |
| Folding endurance (logT) | 2.31 | 2.33 | 2.19 |

Note)
Control means a pulp sheet produced by a woodchip treatment wherein no microorganism treatment was carried out.

### Example 54

### Selection of Transformant Having Suppressed Activity of Engoglucanase Belonging to Glycolytic Enzyme Family 12

Transformation was carried out as described in the transformation method in the above Example 22 using the plasmid pGPEG12AM produced in Example 19 instead of the plasmid pGPCDH1. The obtained transformant was cultured by the same method as in Example 23. The culture solution was sampled with time, and the carboxymethyl cellulose (CMC)-decomposing activity was measured. As a result, it was found that a transformant could be obtained, which had a suppressed endoglucanase activity that was approximately 50% less than that of the host cells.

### Example 55

### Treatment of Woodchips with Transformant Having Suppressed Activity of Endoglucanase Belonging to Glycolytic Enzyme Family 12

Woodchips made from Eucalyptus lumbers were treated with the transformant strain having a suppressed activity of endoglucanase belonging to glycolytic enzyme family 12 selected in Example 54 according to the method described in Example 25.

### Example 56

### Production of Mechanical Pulp Using Transformant Having Suppressed Activity of Endoglucanase Belonging to Glycolytic Enzyme Family 12

Woodchips made from radiata pine lumbers were treated with the transformant obtained in Example 54 in the same manner as in Example 25. Analysis of the paper strength or the like was carried out on the treated woodchips by the method described in Example 26. As shown in Table 21 below, use of a transformant with a suppressed activity of endoglucanase belonging to glycolytic enzyme family 12 could control reduction in chip yield and could reduce refining energy. In addition, both tearing strength and bursting strength were increased. In contrast, when woodchips were treated with a wild type strain, effects of reducing refining energy could be obtained, but paper strength was decreased.

TABLE 21
Effects of woodchip treatment with microorganisms
having suppressed activity of endoglucanase belonging
to glycolytic enzyme family 12 on mechanical pulp

| | | | Wild type |
|---|---|---|---|
| | Control | Transformant | strain |
| Chip yield (%) | 99.8 | 98.5 | 94.7 |
| Refining energy (Kw · h/ton) | 2560 | 1860 | 1840 |
| Tear index (mN · m²/g) | 7.92 | 8.06 | 6.95 |
| Burst index (kPa · m²/g) | 1.35 | 1.43 | 1.21 |

### Example 57

### Cooking of Woodchips Treated with Transformant Having Suppressed Activity of Endoglucanase Belonging to Glycolytic Enzyme Family 12

The woodchips obtained in Example 55 were cooked by the same method as in Example 27.

### Example 58

### Measurement of Ka Value, Preparation of Handsheets Used for Physical Tests of Pulp, and Physical Tests of Handmade Pulp Sheets

Measurement of the kappa value or the like was carried out on the pulp obtained in Example 56 in the same manner as described in Example 28. As shown in Table 22 below, when woodchips were treated with a transformant or wild type strain, the Ka value was decreased after cooking, the Screened yield was increased, and the Screened rejects was decreased. In addition, as shown in Table 23 below, when the transformant was compared with the wild type strain, the transformant did not cause decrease in paper strength.

TABLE 22
Effects of woodchip treatment with microorganisms having
suppressed activity of endoglucanase belonging to glycolytic
enzyme family 12 on cooking

| | Control | Transformant | Wild type strain |
|---|---|---|---|
| Ka value after cooking | 20.1 | 18.2 | 17.7 |
| Screened yield (%) | 45.7 | 47.1 | 47.3 |
| Screened rejects (%) | 1.20 | 0.93 | 0.84 |

TABLE 23
Effects of woodchip treatment with microorganisms having
suppressed activity of endoglucanase belonging to glycolytic
enzyme family 12 on paper strength
Whiteness degree = 86, and CSF (Canadian Shopper
Freeness) = 410 ml

| | Control | Transformant | Wild type strain |
|---|---|---|---|
| PFI (rev) | 2,600 | 2,400 | 2,200 |
| Tear index (mN · m²/g) | 9.4 | 9.3 | 8.5 |
| Breaking length (km) | 8.62 | 8.58 | 7.34 |
| Burst index (kPa · m²/g) | 6.76 | 7.63 | 7.54 |
| Folding endurance (logT) | 2.31 | 2.31 | 2.19 |

Note)
Control means a pulp sheet produced by a woodchip treatment wherein no microorganism treatment was carried out.

### Example 59

### Selection of Transformant Having Suppressed Activity of Engoglucanase Belonging to Glycolytic Enzyme Family 5

Transformation was carried out as described in the transformation method in the above Example 22 using the plasmid pGPPCEG5AM produced in Example 20 instead of the plasmid pGPCDH1. The obtained transformant was cultured by the same method as in Example 23. The culture solution was sampled with time, and the carboxymethyl cellulose (CMC)-decomposing activity was measured. As a result, it was found that a transformant could be obtained, which had a suppressed endoglucanase activity that was approximately 20% less than that of the host cells.

### Example 60

### Treatment of Woodchips with Transformant Having Suppressed Activity of Endoglucanase Belonging to Glycolytic Enzyme Family 5

Woodchips made from Eucalyptus lumbers were treated with the transformant strain having a suppressed activity of endoglucanase belonging to glycolytic enzyme family 5 selected in Example 59 according to the method described in Example 25.

### Example 61

### Production of Mechanical Pulp Using Transformant Having Suppressed Activity of Endoglucanase Belonging to Glycolytic Enzyme Family 5

Woodchips made from radiata pine lumbers were treated with the transformant obtained in Example 59 in the same manner as in Example 25. Analysis of the paper strength or the like was carried out on the treated woodchips by the method described in Example 26.

As shown in Table 24 below, use of a transformant with a suppressed activity of endoglucanase belonging to glycolytic enzyme family 5 could control reduction in chip yield and could reduce refining energy. In addition, both tearing strength and bursting strength were increased. In contrast, when woodchips were treated with a wild type strain, effects of reducing refining energy could be obtained, but paper strength was decreased.

TABLE 24
Effects of woodchip treatment with microorganisms having
suppressed activity of endoglucanase belonging to glycolytic
enzyme family 5 on mechanical pulp

| | Control | Transformant | Wild type strain |
|---|---|---|---|
| Chip yield (%) | 99.8 | 98.2 | 94.7 |
| Refining energy (Kw · h/ | 2560 | 1910 | 1840 |
| ton) | | | |
| Tear index (mN · m²/g) | 7.92 | 8.05 | 6.95 |
| Burst index (kPa · m²/g) | 1.35 | 1.34 | 1.21 |

### Example 62

### Cooking of Woodchips Treated with Transformant Having Suppressed Activity of Endoglucanase Belonging to Glycolytic Enzyme Family 5

The woodchips obtained in Example 60 were cooked by the same method as in Example 27.

### Example 63

### Measurement of Ka Value, Preparation of Handsheets Used for Physical Tests of Pulp, and Physical Tests of Handmade Pulp Sheets

Measurement of the kappa value or the like was carried out on the pulp obtained in Example 62 in the same manner as described in Example 28. As shown in Table 25 below, when woodchips were treated with a transformant or wild type strain, the Ka value was decreased after cooking, the Screened yield was increased, and the Screened rejects was decreased. In addition, as shown in Table 26 below, when the transformant was compared with the wild type strain, the transformant did not cause decrease in paper strength.

TABLE 25
Effects of woodchip treatment with microorganisms having
suppressed activity of endoglucanase belonging to glycolytic
enzyme family 5 on cooking

| | Control | Transformant | Wild type strain |
|---|---|---|---|
| Ka value after cooking | 20.1 | 17.9 | 17.7 |
| Screened yield (%) | 45.7 | 46.8 | 47.3 |
| Screened rejects (%) | 1.20 | 0.95 | 0.84 |

TABLE 26
Effects of woodchip treatment with microorganisms having
suppressed activity of endoglucanase belonging to glycolytic
enzyme family 5 on paper strength
Whiteness degree = 86,and CSF (Canadian Shopper
Freeness) = 410 ml

| | Control | Transformant | Wild type strain |
|---|---|---|---|
| PFI (rev) | 2,600 | 2,300 | 2,200 |
| Tear index (mN · m²/g) | 9.4 | 9.4 | 8.5 |
| Breaking length (km) | 8.62 | 8.55 | 7.34 |
| Burst index (kPa · m²/g) | 6.76 | 7.36 | 7.54 |
| Folding endurance (logT) | 2.31 | 2.26 | 2.19 |

Note)
Control means a pulp sheet produced by a woodchip treatment wherein no microorganism treatment was carried out.

### Example 64

### Selection of Transformant Having Suppressed Activity of Engoglucanase Belonging to Glycolytic Enzyme Family 9

Transformation was carried out as described in the transformation method in the above Example 22 using the plasmid pGPPCEG9AM produced in Example 21 instead of the plasmid pGPCDH1. The obtained transformant was cultured by the same method as in Example 23. The culture solution was sampled with time, and the carboxymethyl cellulose (CMC)-decomposing activity was measured. As a result, it was found that a transformant could be obtained, which had a suppressed endoglucanase activity that was approximately 30% less than that of the host cells.

### Example 65

### Treatment of Woodchips with Transformant Having Suppressed Activity of Endoglucanase Belonging to Glycolytic Enzyme Family 9

Woodchips made from Eucalyptus lumbers were treated with the transformant strain having a suppressed activity of endoglucanase belonging to glycolytic enzyme family 9 selected in Example 64 according to the method described in Example 25.

### Example 66

### Production of Mechanical Pulp Using Transformant Having Suppressed Activity of Endoglucanase Belonging to Glycolytic Enzyme Family 9

Woodchips made from radiata pine lumbers were treated with the transformant obtained in Example 64 in the same manner as in Example 25. Analysis of the paper strength or the like was carried out on the treated woodchips by the method described in Example 26.

As shown in Table 27 below, use of a transformant with a suppressed activity of endoglucanase belonging to glycolytic enzyme family 9 could control reduction in chip yield and could reduce refining energy. In addition, both tearing strength and bursting strength were increased. In contrast, when woodchips were treated with a wild type strain, effects of reducing refining energy could be obtained, but paper strength was decreased.

TABLE 27
Effects of woodchip treatment with microorganisms having
suppressed activity of endoglucanase belonging to glycolytic
enzyme family 9 on mechanical pulp

| | Control | Transformant | Wild type strain |
|---|---|---|---|
| Chip yield (%) | 99.8 | 98.1 | 94.7 |
| Refining energy (Kw · h/ | 2560 | 1860 | 1840 |
| ton) | | | |
| Tear index (mN · m²/g) | 7.92 | 7.98 | 6.95 |
| Burst index (kPa · m²/g) | 1.35 | 1.36 | 1.21 |

### Example 67

### Cooking of Woodchips Treated with Transformant Having Suppressed Activity of Endoglucanase Belonging to Glycolytic Enzyme Family 9

The woodchips obtained in Example 65 were cooked by the same method as in Example 27.

### Example 68

### Measurement of Ka Value, Preparation of Handsheets Used for Physical Tests of Pulp, and Physical Tests of Handmade Pulp Sheets

Measurement of the kappa value or the like was carried out on the pulp obtained in Example 67 in the same manner as described in Example 28. As shown in Table 28 below, when woodchips were treated with a transformant or wild type strain, the Ka value was decreased after cooking, the Screened yield was increased, and the Screened rejects was decreased. In addition, as shown in Table 29 below, when the transformant was compared with the wild type strain, the transformant did not cause decrease in paper strength.

TABLE 28
Effects of woodchip treatment with microorganisms having
suppressed activity of endoglucanase belonging to glycolytic
enzyme family 9 on cooking

| | Control | Transformant | Wild type strain |
|---|---|---|---|
| Ka value after cooking | 20.1 | 18.1 | 17.7 |
| Screened yield (%) | 45.7 | 46.7 | 47.3 |
| Screened rejects (%) | 1.20 | 0.86 | 0.84 |

TABLE 29
Effects of woodchip treatment with microorganisms having
suppressed activity of endoglucanase belonging to glycolytic
enzyme family 9 on paper strength
Whiteness degree = 86, and CSF (Canadian Shopper
Freeness) = 410 ml

| | Control | Transformant | Wild type strain |
|---|---|---|---|
| PFI (rev) | 2,600 | 2,200 | 2,200 |
| Tear index (mN · m²/g) | 9.4 | 9.6 | 8.5 |
| Breaking length (km) | 8.62 | 8.44 | 7.34 |
| Burst index (kPa · m²/g) | 6.76 | 7.45 | 7.54 |
| Folding endurance (logT) | 2.31 | 2.25 | 2.19 |

Note)
Control means a pulp sheet produced by a woodchip treatment wherein no microorganism treatment was carried out.

### Example 69

### Cellobiose Dehydrogenase Activity

1. Summary of Measurement Method

Cellobiose dehydrogenase activity was measured as follows. A solution was produced by mixing 250 ul of 0.67 mM dichlorophenolindophenol (manufactured by Sigma Chemical Company), 100 ul of 3.33 mM cellobiose (manufactured by Kanto Kagaku), and 100 ul of a 250 mM acetate buffer solution of pH 5, and thereafter, 50 ul of a test solution was added to the mixed solution, followed by reaction at 37° C. After initiation of the reaction, absorbance (optical length: 1 cm) at 550 nm (molar absorption coefficient: 3965 L/mol/cm) as the maximum absorption wavelength of dichlorophenolindophenol, was continuously measured. With regard to the activity unit of cellobiose dehydrogenase, the amount of enzyme necessary for decreasing 1 mmol dichlorophenolindophenol per minute under the above conditions was defined as 1 unit (unit: U).

2. Preparation of Crude Enzyme Solution (1)

100 ml of a liquid medium (pH 5.0) containing 1.0% hardwood oxygen-bleached kraft pulp (kappa value: 8.5, whiteness degree: 46.0%), 1.0% peptone, 0.005% MgSO₄.7H₂O, 0.15% KH₂PO₄, and 20 ppb thiamine hydrochloride was placed in a 500 ml-volume Erlenmeyer flask, and the flask was then closed with a paper, followed by steam sterilization at 121° C. for 15 minutes. A Coriolus hirsutus IFO4917 strain was inoculated into the resultant product using an inoculating loop, followed by a rotary shaking culture at 27° C. (amplitude: 25 mm, 120 reciprocations/minute). After completion of the culture, the culture product was subjected to centrifugation (10,000 rpm×10 minutes) to separate the culture supernatant, thereby obtaining a crude enzyme solution of cellobiose dehydrogenase. The activity of the enzyme was measured under the above conditions. As a result, it was found that the cellobiose dehydrogenase activity in the culture supernatant was 0.06 U/ml at 72 hours after initiation of the culture.

3. Preparation of Crude Enzyme Solution (2)

100 ml of a liquid medium (pH 5.0) containing 1.0% Avicel (manufactured by Funakoshi Co., Ltd.), 1.0% peptone, 0.005%, 0.15% KH₂PO₄, and 20 ppb thiamine hydrochloride was placed in a 500 ml-volume Erlenmeyer flask, and the flask was then closed with a paper, followed by steam sterilization at 121° C. for 15 minutes. A Coriolus hirsutus IFO4917 strain was inoculated into the resultant product using an inoculating loop, followed by a rotary shaking culture at 27° C. (amplitude: 25 mm, 120 reciprocations/minute). After completion of the culture, the culture product was subjected to centrifugation (10,000 rpm×10 minutes) to separate the culture supernatant, thereby obtaining a crude enzyme solution of cellobiose dehydrogenase. The activity of the enzyme was measured under the above conditions. As a result, it was found that the cellobiose dehydrogenase activity in the culture supernatant was 0.07 U/ml at 72 hours after initiation of the culture.

4. Purification of Cellobiose Dehydrogenase (1)

The crude enzyme solution obtained in Example 1 or 2 was subjected to ammonium sulfate fractionation, and 80% deposit fraction was then recovered by centrifugation (20,000 rpm×10 minutes). Thereafter, the obtained fraction was dissolved in a 20 mM phosphate buffer solution (pH 6.0). The obtained crude enzyme solution was subjected to hydrophobic chromatography, using Resource 15PHE (diameter 1.6×3 cm; manufactured by Amersham) equilibrated with a 20 mM phosphate buffer solution (pH 6.0) containing 1 M ammonium sulfate.

Adsorption fractions were eluted with a 20 mM phosphate buffer solution (pH 6.0) in a concentration gradient of ammonium sulfate from 1 M to 0 M. The obtained fractions were fractionated into 9 ml fractions, and thus, active fractions were obtained. The obtained fractions were then subjected to gel filtration chromatography, using HiLoad26/60 Superdex 200 (diameter 2.6×60 cm; manufactured by Amersham) equilibrated with a 20 mM phosphate buffer solution (pH 6.0) containing 100 mM sodium chloride. The chromatography was carried out at a flow rate of 1.5 ml/minute, the samples were fractionated into 3 ml fractions, and the active fractions were obtained.

These fractions were collected and subjected to ion exchange chromatography, using POROS HQ (diameter 4.6×10 cm; manufactured by ABI) equilibrated with a 20 mM phosphate buffer solution (pH 6.0). Adsorbed fractions were eluted with the above buffer solution containing sodium chloride in a cosncentration gradient from 0 M to 1 M. The obtained fractions were fractionated into 9 ml fractions, and thus, active fractions were obtained.

The above active fractions were subjected to SDS polyacrylamide electrophoresis. As a result, it could be confirmed that the fractions were uniformly purified. The yield of the purified enzyme was 4.9% with respect to the culture solution, and the specific activity was. 10.5 U/mg.

5. Purification of Cellobiose Dehydrogenase (2)

When the crude enzyme solution obtained in Example 1 or 2 were frozen and then melted, glucan-like substances were deposited. These glucan-like substances were eliminated by centrifugation (20,000 rpm×10 minutes), and ammonium sulfate was then added such that the concentration of ammonium sulfate became 1 M. The obtained crude enzyme solution was subjected to hydrophobic chromatography, using Resource 15PHE (diameter 1.6×3 cm; manufactured by Amersham) equilibrated with a 20 mM phosphate buffer solution (pH 6.0) containing 1 M ammonium sulfate.

Adsorption fractions were eluted with a 20 mM phosphate buffer solution (pH 6.0) in a concentration gradient of ammonium sulfate from 1 M to 0 M. The obtained fractions were fractionated into 9 ml fractions, and thus, active fractions were obtained. The obtained fractions were then subjected to gel filtration chromatography, using HiLoad26/60 Superdex 200 (diameter 2.6×60 cm; manufactured by Amersham) equilibrated with a 20 mM phosphate buffer solution (pH 6.0) containing 100 mM sodium chloride. The chromatography was carried out at a flow rate of 1.5 ml/minute, the samples were fractionated into 3 ml fractions, and the active fractions were obtained. These fractions were collected and subjected to ion exchange chromatography, using monoQ HR 5/5 (diameter 0.5×5 cm; manufactured by Amersham) equilibrated with a 20 mM phosphate buffer solution (pH 6.0). Adsorbed fractions were eluted with the above buffer solution containing sodium chloride in a concentration gradient from 0 M to 0.4 M. The obtained fractions were fractionated into 1 ml fractions, and thus, active fractions were obtained.

The above active fractions were subjected to SDS polyacrylamide electrophoresis. As a result, it could be confirmed that the fractions were uniformly purified. The yield of the purified enzyme was 16.6% with respect to the culture solution, and the specific activity was 10.5 U/mg.

### INDUSTRIAL APPLICABILITY

The present invention provides a gene encoding cellulolytic enzyme derived from Basidiomycete, a transformant transformed with a recombinant vector containing the above gene or an antisense gene of the above gene, and a use thereof. Host cells having a suppressed cellulolytic enzyme activity are prepared by genetic recombination using an antisense gene of the above gene encoding cellulolytic enzyme, and the host cells having a suppressed cellulolytic enzyme activity are used in treatment of woodchips, so as to realize a pulp production method that is excellent in yield and paper strength.

All publications, patents and patent applications cited herein are incorporated herein by reference in their entirety.

1. A method for treating woodchips, comprising the steps of:
preparing a DNA encoding an antisense RNA substantially complementary to the whole or a part of a transcription product of a cellulolytic enzyme gene derived from Basidiomycete;
preparing a vector comprising (a) said DNA, or (b) a recombinant DNA comprising said DNA and a DNA fragment having a promoter activity, wherein said DNA binds to said DNA fragment such that an antisense RNA of the cellulolytic enzyme gene is generated as a result of transcription;
transforming host cells with said vector, so as to prepare the host cells having a suppressed cellulolytic enzyme activity; and
inoculating said host cells having a suppressed cellulolytic enzyme activity into woodchips to treat them.

2. The method according to claim 1, wherein the cellulolytic enzyme gene comprises one or more genes selected from the group consisting of respective genes encoding a cellobiose dehydrogenase, cellobiohydrolase I, cellobiohydrolase II, an endoglucanase belonging to glycolytic enzyme family 61, an endoglucanase belonging to glycolytic enzyme family 12, an endoglucanase belonging to glycolytic enzyme family 5, and an endoglucanase belonging to glycolytic enzyme family 9.

3. The method according to claim 2, wherein the cellobiose dehydrogenase gene is an isolated cellobiose dehydrogenase gene comprising any one of the following nucleotide sequences (a) to (c):
(a) a nucleotide sequence as shown in SEQ ID No. 1 or 3;
(b) a nucleotide sequence hybridizing with a nucleotide sequence comprising a nucleotide sequence complementary to the nucleotide sequence according to (a) under stringent conditions, and encoding a protein having a cellobiose dehydrogenase enzyme activity; and
(c) a nucleotide sequence comprising deletion, substitution or addition of one or more nucleotides with respect to SEQ ID No. 1 or 3, and encoding a protein having a cellobiose dehydrogenase enzyme activity.

4. The method according to claim 2, wherein the cellobiohydrolase I gene is an isolated cellobiohydrolase I gene comprising any one of the following nucleotide sequences (a) to (c):
(a) a nucleotide sequence as shown in SEQ ID No. 7, 9, or 11;
(b) a nucleotide sequence hybridizing with a nucleotide sequence comprising a nucleotide sequence complementary to the nucleotide sequence according to (a) under stringent conditions, and encoding a protein having a cellobiohydrolase I gene enzyme activity; and
(c) a nucleotide sequence comprising deletion, substitution or addition of one or more nucleotides with respect to SEQ ID No. 7, 9 or 11, and encoding a protein having a cellobiohydrolase I gene enzyme activity.

5. The method according to claim 2, wherein the cellobiohydrolase 11 gene is an isolated cellobiohydrolase 11 gene comprising any one of the following nucleotide sequences (a) to (c):
(a) a nucleotide sequence as shown in SEQ ID No. 14;
(b) a nucleotide sequence hybridizing with a nucleotide sequence comprising a nucleotide sequence complementary to the nucleotide sequence according to (a) under stringent conditions, and encoding a protein having a cellobiohydrolase 11 gene enzyme activity; and
(c) a nucleotide sequence comprising deletion, substitution or addition of one or more nucleotides with respect to SEQ ID No. 14, and encoding a protein having a cellobiohydrolase II gene enzyme activity.

6. The method according to claim 2, wherein the endoglucanase gene belonging to glycolytic enzyme family 61 is an isolated endoglucanase gene belonging to glycolytic enzyme family 61 comprising any one of the following nucleotide sequences (a) to (c):
(a) a nucleotide sequence as shown in SEQ ID No. 18;
(b) a nucleotide sequence hybridizing with a nucleotide sequence comprising a nucleotide sequence complementary to the nucleotide sequence according to (a) under stringent conditions, and encoding a protein having an activity of endoglucanase enzyme belonging to glycolytic enzyme family 61; and
(c) a nucleotide sequence comprising deletion, substitution or addition of one or more nucleotides with respect to SEQ ID No. 18, and encoding a protein having an activity of endoglucanase enzyme belonging to glycolytic enzyme family 61.

7. The method according to claim 2, wherein the endoglucanase gene belonging to glycolytic enzyme family 12 is an isolated endoglucanase gene belonging to glycolytic enzyme family 12 comprising any one of the following nucleotide sequences (a) to (c):
(a) a nucleotide sequence as shown in SEQ ID No. 20;
(b) a nucleotide sequence hybridizing with a nucleotide sequence comprising a nucleotide sequence complementary to the nucleotide sequence according to (a) under stringent conditions, and encoding a protein having an activity of endoglucanase enzyme belonging to glycolytic enzyme family 12; and
(c) a nucleotide sequence comprising deletion, substitution or addition of one or more nucleotides with respect to SEQ ID No. 20, and encoding a protein having an activity of endoglucanase enzyme belonging to glycolytic enzyme family 12.

8. The method according to claim 2, wherein the endoglucanase gene belonging to glycolytic enzyme family 5 is an isolated endoglucanase gene belonging to glycolytic enzyme family 5 comprising any one of the following nucleotide sequences (a) to (c):
(a) a nucleotide sequence as shown in SEQ ID No. 24;
(b) a nucleotide sequence hybridizing with a nucleotide sequence comprising a nucleotide sequence complementary to the nucleotide sequence according to (a) under stringent conditions, and encoding a protein having an activity of endoglucanase enzyme belonging to glycolytic enzyme family 5; and
(c) a nucleotide sequence comprising deletion, substitution or addition of one or more nucleotides with respect to SEQ ID No. 24, and encoding a protein having an activity of endoglucanase enzyme belonging to glycolytic enzyme family 5.

9. The method according to claim 2, wherein the endoglucanase gene belonging to glycolytic enzyme family 9 is an isolated endoglucanase gene belonging to glycolytic enzyme family 9 comprising any one of the following nucleotide sequences (a) to (c):
(a) a nucleotide sequence as shown in SEQ ID No. 28;
(b) a nucleotide sequence hybridizing with a nucleotide sequence comprising a nucleotide sequence complementary to the nucleotide sequence according to (a) under stringent conditions, and encoding a protein having an activity of endoglucanase enzyme belonging to glycolytic enzyme family 9; and
(c) a nucleotide sequence comprising deletion, substitution or addition of one or more nucleotides with respect to SEQ ID No. 28, and encoding a protein having an activity of endoglucanase enzyme belonging to glycolytic enzyme family 9.

10. The method according to claim 1, wherein Basidiomycete is Coriolus hirsutus or Phanerochaete chrysosporium.

11. The method according to claim 1, wherein host cells are Coriolus hirsutus.

12. A woodchip obtained by the method according to claim 1.

13. A method for producing a pulp using the woodchip according to claim 12.

14. A pulp obtained by the method according to claim 13.
